(19) United States
(12) Patent Application Publication
(10) Pub. No.: US 2023/0092707 A1
(43) Pub. Date: Mar. 23, 2023

(54) METHODS FOR TREATING CANCER OR INFECTION USING A COMBINATION OF AN ANTI-PD-1 ANTIBODY, AN ANTI-CTLA4 ANTIBODY, AND AN ANTI-TIGIT ANTIBODY

(71) Applicant: Merck Sharp & Dohme LLC, Rahway, NJ (US)

(72) Inventors: Rachel A. Altura, Belle Mead, NJ (US); Mingmei Cai, Westwood, MA (US); Scott J. Diede, New Hope, PA (US); Jane Anne Healy, Hoboken, NJ (US); Blanca Homet Moreno, Philadelphia, PA (US); Nageatte Ibrahim, Plymouth Meeting, PA (US); Sybil M.G. Williams, Wayland, MA (US)

(73) Assignee: MERCK SHARP & DOHME LLC, Rahway, NJ (US)

(21) Appl. No.: 17/801,454

(22) PCT Filed: Mar. 4, 2021
(86) PCT No.: PCT/US2021/020781

§ 371 Date: Aug. 22, 2022

Related U.S. Application Data
(60) Provisional application No. 63/043,888, filed on Jun. 25, 2020.
(60) Provisional application No. 62/985,645, filed on Mar. 5, 2020.

(51) Int. Cl. C07K 16/28; C07K 16/30; A61P 35/00

(52) U.S. Cl. CPC C07K 16/2818; C07K 16/30; A61P 35/00; C07K 2317/73; C07K 2317/24; A61K 2039/507

(57) ABSTRACT

Provided herein are methods of treating a cancer, an infectious disease or infection, which comprise administering to a subject in need thereof a combination of agents: (a) an anti-human PD-1 antibody or antigen binding fragment thereof; (b) an anti-human CTLA4 antibody or antigen binding fragment thereof; and (c) an anti-human TIGIT antibody or antigen binding fragment thereof. Also provided are therapeutic combinations for use, pharmaceutical compositions and kits containing such agents for the treatment of a cancer, an infectious disease, or an infection.

### RELATED APPLICATIONS

This application claims the benefit of U.S. Provisional Patent Application No. 62/985,645, filed Mar. 5, 2020, and U.S. Provisional Patent Application No. 63/043,888, filed Jun. 25, 2020, each of which is incorporated by reference herein in its entirety.

### REFERENCE TO SEQUENCE LISTING SUBMITTED ELECTRONICALLY

The present specification is being filed with a computer readable form (CRF) copy of the Sequence Listing. The CRF entitled 24843WOPCT-SEQLIST-01FEB2021.txt which was created on Feb. 1, 2021 and is 72.3 kilobytes in size, is incorporated herein by reference in its entirety.

### FIELD OF THE INVENTION

Provided herein are methods for treating cancer or an infectious disease using a combination of (a) an antibody that binds to a Programmed Death 1 protein (PD-1) or antigen binding fragment thereof, (b) an antibody that binds to a cytotoxic T-lymphocyte-associated antigen 4 (CTLA4; also known as cytotoxic T-lymphocyte-associated protein 4) or antigen binding fragment thereof, and (c) an antibody that binds to a T cell immunoreceptor with Ig and ITIM domains (TIGIT) or antigen binding fragment thereof.

### BACKGROUND OF THE INVENTION

As with chronic viral infection, tumor antigen-specific CD4+ and CD8+ T cells display impaired effector function and an exhausted phenotype characterized by decreased production of pro-inflammatory cytokines and hypo-responsiveness to antigenic re-stimulation. This is mediated by cell extrinsic mechanisms, such as regulatory T cells (Treg), and cell intrinsic mechanisms, such as inhibitory molecules that are upregulated on exhausted, tumor-infiltrating lymphocytes. These inhibitory mechanisms represent a formidable barrier to effective antitumor immunity.

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372: 2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA© (pembrolizumab), Merck and Co., Inc., Kenilworth, N.J.; OPDIVO© (nivolumab), Bristol-Myers Squibb Company, Princeton, N.J.) and those that bind to the PD-L1 ligand (e.g., TECENTRIQ© (atezolizumab), Genentech, San Francisco, Calif.).

It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. One such agent that has been tested in combination with antagonists of PD-1 is an antagonist of CTLA4.

It has been proposed that the efficacy of anti-PD-1 or anti-PD-L1 antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. However, there are no clear guidelines as to which agent combined with the anti-PD-1 or anti-PD-L1 antibodies may be effective or in which cancer types the combination may enhance the efficacy of treatment. Thus, there is an unmet need in the art for high efficacy therapeutic combinations that can generate a robust immune response to cancer.

### SUMMARY OF THE INVENTION

The present disclosure provides methods, pharmaceutical compositions, uses and kits of treating a cancer, an infectious disease, or an infection using a combination of therapeutic agents, e.g., a combination of antibodies or antigen binding fragments thereof.

The present disclosure provides methods of treating a cancer (e.g., colorectal cancer), an infectious disease (e.g., a viral infection), or an infection using a combination of an anti-PD-1 antibody (e.g., monoclonal antibody) or antigen binding fragment thereof, an anti-CTLA4 antibody (e.g., monoclonal antibody) or antigen binding fragment thereof, and an anti-TIGIT antibody (e.g., monoclonal antibody) or antigen binding fragment thereof.

The present disclosure also provides pharmaceutical compositions comprising an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof.

The present disclosure further provides kits including an anti-PD-1 antibody (e.g., monoclonal antibody) or antigen binding fragment thereof, an anti-CTLA4 antibody (e.g., monoclonal antibody) or antigen binding fragment thereof, and an anti-TIGIT antibody (e.g., monoclonal antibody) or antigen binding fragment thereof.

Also provided herein are uses of a therapeutic combination for treating cancer (e.g., colorectal cancer), and the therapeutic combination includes an anti-PD-1 antibody (e.g., monoclonal antibody) or antigen binding fragment thereof, an anti-CTLA4 antibody (e.g., monoclonal antibody) or antigen binding fragment thereof, and an anti-TIGIT antibody (e.g., monoclonal antibody) or antigen binding fragment thereof.

Also provided herein are therapeutic combinations for use in treating cancer (e.g., colorectal cancer), and the therapeutic combination includes an anti-PD-1 antibody (e.g., monoclonal antibody) or antigen binding fragment thereof, an anti-CTLA4 antibody (e.g., monoclonal antibody) or antigen binding fragment thereof, and an anti-TIGIT antibody (e.g., monoclonal antibody) or antigen binding fragment thereof.

In various embodiments of the methods, pharmaceutical compositions, kits, uses, or the combinations for use, the anti-PD-1 antibody (e.g., monoclonal antibody) or antigen binding fragment thereof, and/or the anti-CTLA4 antibody (e.g., monoclonal antibody) or antigen binding fragment thereof, and/or the anti-TIGIT antibody (e.g., monoclonal antibody) or antigen binding fragment thereof is administered using a regimen that encompasses administering at least one of the antibodies during specific hours, days or weeks.

In various embodiments of the methods, pharmaceutical compositions, kits, uses, or the combinations for use, the anti-PD-1 antibody (e.g., monoclonal antibody) or antigen binding fragment thereof, and/or the anti-CTLA4 antibody (e.g., monoclonal antibody) or antigen binding fragment thereof, and/or the anti-TIGIT antibody (e.g., monoclonal antibody) or antigen binding fragment thereof is administered Q5D, Q7D, or Q10D.

In various embodiments of the methods, pharmaceutical compositions, kits, uses, or the combinations for use, the anti-PD-1 antibody (e.g., monoclonal antibody) or antigen binding fragment thereof, and/or the anti-CTLA4 antibody (e.g., monoclonal antibody) or antigen binding fragment thereof, and/or the anti-TIGIT antibody (e.g., monoclonal antibody) or antigen binding fragment thereof is administered every day, every 2 days, every 3 days, every 4 days, every 5 days, every 6 days, every 7 days, every 8 days, every 9 days, or every 10 days.

In various embodiments of the methods, pharmaceutical compositions, kits, uses, or the combinations for use, the anti-PD-1 antibody (e.g., monoclonal antibody) or antigen binding fragment thereof, and/or the anti-CTLA4 antibody (e.g., monoclonal antibody) or antigen binding fragment thereof, and/or the anti-TIGIT antibody (e.g., monoclonal antibody) or antigen binding fragment thereof is administered at a dose of at least 0.5 mpk, at least 1 mpk, at least 2 mpk, at least 3 mpk, at least 4 mpk, at least 5 mpk, at least 6 mpk, at least 7 mpk, at least 8 mpk, at least 9 mpk, at least 10 mpk, at least 11 mpk, at least 12 mpk, at least 13 mpk, at least 14 mpk, or at least 15 mpk.

In various embodiments of the methods, pharmaceutical compositions, kits, uses, or the combinations for use, the anti-PD-1 antibody or antigen binding fragment thereof is administered at a dose of 5 mpk. In various embodiments, the anti-PD-1 antibody or antigen binding fragment thereof is administered Q5D.

In various embodiments of the methods, pharmaceutical compositions, kits, uses, or the combinations for use, the anti-TIGIT antibody or antigen binding fragment thereof is administered at a dose of 10 mpk. In various embodiments, the anti-TIGIT antibody or antigen binding fragment thereof is administered Q5D.

In various embodiments of the methods, pharmaceutical compositions, kits, uses, or the combinations for use, the anti-CTLA4 antibody or antigen binding fragment thereof is administered at a dose of 1 mpk. In various embodiments, the anti-CTLA4 antibody or antigen binding fragment thereof is administered at a dose of 0.5 mpk. In various embodiments, the anti-CTLA4 antibody or antigen binding fragment thereof is administered Q5d. In various embodiments, the anti-CTLA4 antibody or antigen binding fragment thereof is administered Q10D.

In one aspect, provided herein is a method of treating cancer, an infection disease or an infection comprising administering to a subject (e.g., a human patient or a murine subject) in need thereof:
(a) an effective amount of an anti-human PD-1 antibody (e.g., monoclonal antibody) or antigen binding fragment thereof,
(b) an effective amount of an anti-human CTLA4 antibody (e.g., monoclonal antibody) or antigen binding fragment thereof, and
(c) an effective amount of an anti-human TIGIT antibody (e.g., monoclonal antibody) or antigen binding fragment thereof.

In various embodiments of the method, the anti-PD-1 antibody (e.g., monoclonal antibody) or antigen binding fragment is administered via intravenous infusion.

In various embodiments of the method, the anti-CTLA4 antibody (e.g., monoclonal antibody) or antigen binding fragment is administered via intravenous infusion.

In various embodiments of the method, the anti-TIGIT antibody (e.g., monoclonal antibody) or antigen binding fragment is administered via intravenous infusion.

In various embodiments of the method, the administering of one or more of the anti-PD-1, anti-CTLA4 and anti-TIGIT is performed by injection. In various embodiments of the methods, the administering is performed by infusion.

In various embodiments of the method, the administering of (a), (b), and (c) is performed sequentially. In various embodiments of the method, (a) is administered, then (b) and (c). In various embodiments, (a) is administered, then (c) and (b). In various embodiments, (b) is administered, then (a) and (c). In various embodiments, (b) is administered, then (c) and (a). In various embodiments, (c) is administered, then (a) and (b). In various embodiments, (c) is administered, then (b) and (c).

Alternatively, in various embodiments of the method, the administering of (a), (b), and (c) is performed concurrently.

In various embodiments, the cancer is a solid tumor. In various embodiments of the method, the cancer is selected from a cancer disclosed in the Examples. In various embodiments of the method, the cancer is at least one from the group consisting of: non-small cell lung cancer (NSCLC), colorectal cancer, cervical cancer, gastric cancer, breast cancer, ovarian, epithelial, fallopian tube, or primary peritoneal carcinoma. For example, the cancer is NSCLC. In various embodiments, the subject or patient has a cancer and expresses at least one Breast Cancer gene (e.g., BRCA). In various embodiments, the cancer or a sample from the subject is found to have a level or to express at least one Breast Cancer gene (BRCA). In various embodiments, the at least one BRCA gene is BRCA1 or BRCA2. In an embodiment, the cancer is BRCA negative. For example, the cancer (for example breast cancer and ovarian cancer) is a BRCA negative cancer. In an embodiment, the cancer is BRCA positive.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer. For example, the cancer is a melanoma. In certain embodiments, the cancer is a treatment of PD-1 refractory melanoma. In yet another embodiment, the subject has melanoma brain metastases. In yet another embodiment, the cancer is refractory melanoma.

In certain embodiments, the cancer is metastatic. In some embodiments, the cancer is relapsed. In other embodiments, the cancer is refractory. In yet other embodiments, the cancer is relapsed and refractory. For example, the relapsed and refractory cancer is any of the cancers described herein.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In yet still another embodiment, the cancer is PD-1 refractory rrNHL. For example, the cancer is a melanoma. In certain embodiments, the cancer is a treatment of PD-1 refractory melanoma. In yet another embodiment, the cancer is melanoma and the subject (e.g., human patient) has melanoma brain metastases. In yet another embodiment, the cancer is refractory melanoma. In various embodiments, the cancer is locally advanced or resectable. In various embodiments, the cancer has been treated with an adjuvant. In various embodiments, the cancer has been treated with a neoadjuvant.

In another aspect, provided herein is a method of enhancing T cell activity, comprising contacting the T cells with:
(a) an anti-human PD-1 antibody (e.g., monoclonal antibody) or antigen binding fragment thereof,
(b) an anti-human CTLA4 antibody (e.g., monoclonal antibody) or antigen binding fragment thereof, and
(c) an anti-human TIGIT antibody (e.g., monoclonal antibody) or antigen binding fragment thereof.

In some embodiments, the enhancement of T cell activity occurs in vitro. In other embodiments, the enhancement of T cell activity occurs in vivo. For example, the enhancement is in a subject including but not limited to a human subject or human patient.

In certain embodiments, the enhancement of T cell activity is measured by increased cytokine production. In other embodiments, the enhancement of T cell activity is measured by increased cell proliferation.

In some embodiments, provided herein is a method of increasing cytokine production of T cells, comprising contacting the T cells with:
(a) an anti-human PD-1 antibody (e.g., monoclonal antibody) or antigen binding fragment thereof,
(b) an anti-human CTLA4 antibody (e.g., monoclonal antibody) or antigen binding fragment thereof, and
(c) an anti-human TIGIT antibody (e.g., monoclonal antibody) or antigen binding fragment thereof.

In some embodiments, the increased cytokine production of T cells occurs in vitro. In other embodiments, the increased cytokine production of T cells occurs in vivo.

In certain embodiments, the cytokine is selected from the group consisting of IL-1, IL-2, IL-6, IL-12, IL-17, IL-22, IL-23, GM-CSF, IFN-α, and TNF-α. In one embodiment, the cytokine is IL-1. In another embodiment, the cytokine is IL-2. In yet another embodiment, the cytokine is IL-6. In still another embodiment, the cytokine is IL-12. In one embodiment, the cytokine is IL-17. In another embodiment, the cytokine is IL-22. In yet another embodiment, the cytokine is IL-23. In still another embodiment, the cytokine is GM-CSF. In one embodiment, the cytokine is IFN-α. In another embodiment, the cytokine is TNF-α. In some embodiments, the cytokine is one, two, three, four, five, six, seven, eight, nine, or ten cytokines selected from the group consisting of IL-1, IL-2, IL-6, IL-12, IL-17, IL-22, IL-23, GM-CSF, IFN-α, and TNF-α.

In other embodiments, provided herein is a method of increasing proliferation of T cells, comprising contacting the T cells with:
(a) an anti-human PD-1 antibody (e.g., monoclonal antibody) or antigen binding fragment thereof,
(b) an anti-human CTLA4 antibody (e.g., monoclonal antibody) or antigen binding fragment thereof, and
(c) an anti-human TIGIT antibody (e.g., monoclonal antibody) or antigen binding fragment thereof.

In some embodiments, the increased proliferation of T cells occurs in vitro. In other embodiments, the increased proliferation of T cells occurs in vivo.

In another aspect, provided herein is a pharmaceutical composition for treating cancer, an infection disease or an infection in a subject in need thereof comprising:
(a) an anti-human PD-1 antibody (e.g., monoclonal antibody) or antigen binding fragment thereof,
(b) an anti-human CTLA4 antibody (e.g., monoclonal antibody) or antigen binding fragment thereof, and
(c) an anti-human TIGIT antibody (e.g., monoclonal antibody) or antigen binding fragment thereof.

In certain embodiments, the pharmaceutical composition further comprises a pharmaceutically acceptable carrier. In various embodiments, the pharmaceutical composition further comprises at least one additional therapeutic agent and/or active agent.

In yet another aspect, provided herein is a kit for treating cancer, an infection disease or an infection in a subject in need thereof comprising:
(a) an anti-human PD-1 antibody (e.g., monoclonal antibody) or antigen binding fragment thereof,
(b) an anti-human CTLA4 antibody (e.g., monoclonal antibody) or antigen binding fragment thereof, and
(c) an anti-human TIGIT antibody (e.g., monoclonal antibody) or antigen binding fragment thereof.

In certain embodiments, the kit further comprises instructions for administering to a subject (e.g., a human patient) in need thereof effective amounts of the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.

In still another aspect, provided herein is use of a therapeutic combination for treating cancer, an infection disease or an infection in a subject in need thereof in a subject (e.g., a human patient), wherein the therapeutic combination comprises effective amounts of:
(a) an anti-human PD-1 antibody (e.g., monoclonal antibody) or antigen binding fragment thereof,
(b) an anti-human CTLA4 antibody (e.g., monoclonal antibody) or antigen binding fragment thereof, and
(c) an anti-human TIGIT antibody (e.g., monoclonal antibody) or antigen binding fragment thereof.

In various embodiments of the method, the cancer is selected from a cancer disclosed in the Examples. In various embodiments of the method, the cancer is at least one from the group consisting of: NSCLC, colorectal cancer, cervical cancer, gastric cancer, breast cancer, ovarian, epithelial, fallopian tube, or primary peritoneal carcinoma.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., DLBCL or NHL), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory. In various embodiments, the cancer is a melanoma. In certain embodiments, the cancer is a treatment of PD-1 refractory melanoma. In yet another embodiment, the subject has melanoma brain metastases. In yet another embodiment, the cancer is refractory melanoma.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In yet still another embodiment, the cancer is PD-1 refractory rrNHL.

In certain embodiments of various methods, pharmaceutical compositions, kits, uses, or the combinations for use provided herein, the subject is a human patient. In certain embodiments of various methods, pharmaceutical compositions, kits, or uses provided herein, the subject is a murine subject. In certain embodiments of various methods, pharmaceutical compositions, kits, or combinations of use provided herein, the subject is a primate.

In certain embodiments the methods, pharmaceutical compositions, kits, uses, or the combinations for use provided herein are for treating cancer.

In certain embodiments of various methods, pharmaceutical compositions, kits, uses, or the combinations for use provided herein, the anti-human PD-1 monoclonal antibody is a human antibody.

In some embodiments of various methods, pharmaceutical compositions, kits, uses, or the combinations for use provided herein, the anti-human PD-1 monoclonal antibody is a humanized antibody.

In other embodiments of various methods, pharmaceutical compositions, kits, uses, or the combinations for use provided herein, the anti-human CTLA4 monoclonal antibody is a human antibody.

In yet other embodiments of various methods, pharmaceutical compositions, kits, uses, or the combinations for use provided herein, the anti-human CTLA4 monoclonal antibody is a humanized antibody.

In still other embodiments of various methods, pharmaceutical compositions, kits, uses, or the combinations for use provided herein, the anti-human TIGIT monoclonal antibody is a human antibody.

In still other embodiments of various methods, pharmaceutical compositions, kits, uses, or the combinations for use provided herein, the anti-human TIGIT monoclonal antibody is a humanized antibody.

In still other embodiments of various methods, pharmaceutical compositions, kits, uses, or the combinations for use provided herein, the anti-PD-1 antibody is independently selected from pembrolizumab, nivolumab, cemiplimab, sintilimab, tislelizumab, camrelizumab and toripalimab.

In certain embodiments of various methods, pharmaceutical compositions, kits, uses, or the combinations for use provided herein, the anti-human PD-1 monoclonal antibody or antigen binding fragment thereof comprises a light chain variable region (V_{L}) complementarity determining region 1 (CDR1), a V_{L} CDR2, and a V_{L} CDR3 comprising amino acid sequences as set forth in SEQ ID NOS:1, 2, and 3, respectively, and a heavy chain variable region (V_{H}) CDR1, a V_{H} CDR2, and a V_{H} CDR3 comprising amino acid sequences as set forth in SEQ ID NOS:6, 7, and 8, respectively.

In some embodiments of various methods, pharmaceutical compositions, kits, uses, or the combinations for use provided herein, the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various methods, pharmaceutical compositions, kits, uses, or the combinations for use provided herein, the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In one embodiment of various methods, pharmaceutical compositions, kits, uses, or the combinations for use provided herein, the anti-human PD-1 monoclonal antibody or antigen binding fragment thereof is pembrolizumab.

In another embodiment of various methods, pharmaceutical compositions, kits, uses, or the combinations for use provided herein, the anti-human PD-1 monoclonal antibody or antigen binding fragment thereof is nivolumab.

In another embodiment of various methods, pharmaceutical compositions, kits, uses, or the combinations for use provided herein, the anti-human PD-1 monoclonal antibody or antigen binding fragment thereof is cemiplimab.

In yet another embodiment of various methods, pharmaceutical compositions, kits, uses, or the combinations for use provided herein, the anti-human PD-1 monoclonal antibody or antigen binding fragment thereof is pidilizumab (U.S. Pat. No. 7,332,582).

In one embodiment of various methods, pharmaceutical compositions, kits, uses, or the combinations for use provided herein, the anti-human PD-1 monoclonal antibody or antigen binding fragment thereof is AMP-514 (MedImmune LLC, Gaithersburg, Md.).

In another embodiment of various methods, pharmaceutical compositions, kits, uses, or the combinations for use provided herein, the anti-human PD-1 monoclonal antibody or antigen binding fragment thereof is PDR001 (U.S. Pat. No. 9,683,048).

In yet another embodiment of various methods, pharmaceutical compositions, kits, uses, or the combinations for use provided herein, the anti-human PD-1 monoclonal antibody or antigen binding fragment thereof is BGB-A317 (U.S. Pat. No. 8,735,553).

In still another embodiment of various methods, pharmaceutical compositions, kits, uses, or the combinations for use provided herein, the anti-human PD-1 monoclonal antibody or antigen binding fragment thereof is MGA012 (MacroGenics, Rockville, Md.).

In certain embodiments of various methods, pharmaceutical compositions, kits, uses, or the combinations for use provided herein, the anti-human CTLA4 antibody (e.g., monoclonal antibody) or antigen binding fragment thereof comprises a V_{L} CDR1 comprising the amino acid sequence of SEQ ID NO: 14, a V_{L} CDR2 comprising the amino acid sequence of SEQ ID NO: 15, and a V_{L} CDR3 comprising the amino acid sequences as set forth in SEQ ID NOS:16, 17, or 18, and a V_{H} CDR1, a V_{H} CDR2, and a V_{H} CDR3 comprising amino acid sequences as set forth in SEQ ID NOS:11, 12, and 13, respectively.

In certain embodiments of various methods, pharmaceutical compositions, kits, uses, or the combinations for use provided herein, the anti-human CTLA4 antibody (e.g., monoclonal antibody) or antigen binding fragment thereof comprises a V_{L} CDR1 comprising an amino acid sequence comprising 80%, 85%, 90%, 95%, or 99% sequence identity to SEQ ID NO: 14, a V_{L} CDR2 comprising an amino acid sequence 80%, 85%, 90%, 95%, or 99% sequence identity to SEQ ID NO: 15, and a V_{L} CDR3 comprising an amino acid sequences 80%, 85%, 90%, 95%, or 99% sequence identity to SEQ ID NOS:16, 17, or 18, and a V_{H} CDR1, a V_{H} CDR2, and a V_{H} CDR3 comprising amino acid sequences comprising 80%, 85%, 90%, 95%, or 99% sequence identity to SEQ ID NOS:11, 12, and 13, respectively.

In certain embodiments of various methods, pharmaceutical compositions, kits, uses, or the combinations for use provided herein, the anti-human CTLA4 antibody (e.g., monoclonal antibody) or antigen binding fragment thereof comprises a V_{L} CDR1, a V_{L} CDR2, and a V_{L} CDR3 comprising the amino acid sequences as set forth in SEQ ID NOS:54, 55, and 56, respectively, and a V_{H} CDR1, a V_{H} CDR2, and a V_{H} CDR3 comprising amino acid sequences as set forth in SEQ ID NOS:57, 105, and 106, respectively.

In certain embodiments of various methods, pharmaceutical compositions, kits, uses, or the combinations for use provided herein, the anti-human CTLA4 antibody (e.g., monoclonal antibody) or antigen binding fragment thereof comprises a V_{L} CDR1, a V_{L} CDR2, and a V_{L} CDR3 comprising amino acid sequences comprising 80%, 85%, 90%, 95%, or 99% sequence identity to SEQ ID NOS:54, 55, and 56, respectively, and a V_{H} CDR1, a V_{H} CDR2, and a V_{H} CDR3 comprising amino acid sequences comprising 80%, 85%, 90%, 95%, or 99% sequence identity to SEQ ID NOS:57, 105, and 106, respectively.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:20, and a V_{H} region comprising an amino acid sequence as set forth in SEQ ID NO:19.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:42, and a V_{H} region comprising an amino acid sequence as set forth in SEQ ID NO:41.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:44, and a V_{H} region comprising an amino acid sequence as set forth in SEQ ID NO: 43 or SEQ ID NO: 45. For example, the anti-human CTLA4 monoclonal antibody or antigen binding fragment thereof comprises a V_{L} region comprising an amino acid sequence as set forth in SEQ ID NO:44, and a V_{H} region comprising an amino acid sequence as set forth in SEQ ID NO: 45.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:47, and a V_{H} region comprising an amino acid sequence as set forth in SEQ ID NO:46.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:49, and a V_{H} region comprising an amino acid sequence as set forth in SEQ ID NO:48.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:51, and a V_{H} region comprising an amino acid sequence as set forth in SEQ ID NO:50.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:108, and a V_{H} region comprising an amino acid sequence as set forth in SEQ ID NO:107.

In other embodiments of various methods, pharmaceutical compositions, kits, uses, or the combinations for use provided herein, the anti-human CTLA4 antibody (e.g., monoclonal antibody) or antigen binding fragment thereof comprises a light chain comprising or consisting of an amino acid sequence as set forth in SEQ ID NO:53 and a heavy chain comprising or consisting of an amino acid sequence as set forth in SEQ ID NO: 52.

In other embodiments of various methods, pharmaceutical compositions, kits, uses, or the combinations for use provided herein, the anti-human CTLA4 antibody (e.g., monoclonal antibody) or antigen binding fragment thereof comprises a light chain comprising or consisting of an amino acid sequence as set forth in SEQ ID NO:110 and a heavy chain comprising or consisting of an amino acid sequence as set forth in SEQ ID NO:109.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence comprising 80%, 85%, 90%, 95%, or 99% sequence identity to SEQ ID NO:42, and a V_{H} region comprising an amino acid sequence comprising 80%, 85%, 90%, 95%, or 99% sequence identity to SEQ ID NO:41.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence comprising 80%, 85%, 90%, 95%, or 99% sequence identity to SEQ ID NO:44, and a V_{H} region comprising an amino acid sequence comprising 80%, 85%, 90%, 95%, or 99% sequence identity to SEQ ID NO: 43 or SEQ ID NO: 45. For example, in some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence comprising 80%, 85%, 90%, 95%, or 99% sequence identity to SEQ ID NO:44, and a V_{H} region comprising an amino acid sequence comprising 80%, 85%, 90%, 95%, or 99% sequence identity to SEQ ID NO: 45.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence comprising 80%, 85%, 90%, 95%, or 99% sequence identity to SEQ ID NO:47, and a V_{H} region comprising an amino acid sequence comprising 80%, 85%, 90%, 95%, or 99% sequence identity to SEQ ID NO:46.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:49, and a V_{H} region comprising an amino acid sequence comprising 80%, 85%, 90%, 95%, or 99% sequence identity to SEQ ID NO:48.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence as set forth in SEQ ID NO:51, and a V_{H} region comprising an amino acid sequence comprising 80%, 85%, 90%, 95%, or 99% sequence identity to SEQ ID NO:50.

In some embodiments of various methods, pharmaceutical compositions, kits, uses, or the combinations for use provided herein, the anti-human CTLA4 antibody (e.g., monoclonal antibody) or antigen binding fragment thereof comprises a V_{L} region comprising an amino acid sequence comprising 80%, 85%, 90%, 95%, or 99% sequence identity to SEQ ID NO:108, and a V_{H} region comprising an amino acid sequence comprising 80%, 85%, 90%, 95%, or 99% sequence identity to SEQ ID NO:107.

In other embodiments of various methods, pharmaceutical compositions, kits, uses, or the combinations for use provided herein, the anti-human CTLA4 antibody (e.g., monoclonal antibody) or antigen binding fragment thereof comprises a light chain comprising or consisting of an amino acid sequence comprising 80%, 85%, 90%, 95%, or 99% sequence identity to SEQ ID NO:53 and a heavy chain comprising or consisting of an amino acid sequence comprising 80%, 85%, 90%, 95%, or 99% sequence identity to SEQ ID NO:52.

In other embodiments of various methods, pharmaceutical compositions, kits, uses, or the combinations for use provided herein, the anti-human CTLA4 antibody (e.g., monoclonal antibody) or antigen binding fragment thereof comprises a light chain comprising or consisting of an amino acid sequence comprising 80%, 85%, 90%, 95%, or 99% sequence identity to SEQ ID NO:110 and a heavy chain comprising or consisting of an amino acid sequence comprising 80%, 85%, 90%, 95%, or 99% sequence identity to SEQ ID NO:109.

In certain embodiments of various methods, pharmaceutical compositions, kits, uses, or the combinations for use provided herein, the anti-human TIGIT antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments of various methods, pharmaceutical compositions, kits, uses, or the combinations for use provided herein, the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In other embodiments of various methods, pharmaceutical compositions, kits, uses, or the combinations for use provided herein, the anti-human TIGIT antibody (e.g., monoclonal antibody) or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In one embodiment of various methods, pharmaceutical compositions, kits, uses, or the combinations for use provided herein, the anti-human TIGIT antibody (e.g., monoclonal antibody) or antigen binding fragment thereof is described in international patent publication number WO2016/028656 (Merck Sharp & Dohme Corp, Rahway, N.J.). In various embodiments, the anti-human TIGIT monoclonal antibody or antigen binding fragment thereof is a humanized antibody.

In certain embodiments of various methods, pharmaceutical compositions, kits, uses, or the combinations for use provided herein, the anti-human TIGIT antibody (e.g., monoclonal antibody) or antigen binding fragment thereof comprises a V_{L} CDR1, a V_{L} CDR2, and a V_{L} CDR3 comprising amino acid sequences comprising 80%, 85%, 90%, 95%, or 99% sequence identity to SEQ ID NOS:21, 22, and 23, respectively, and a V_{H} CDR1, a V_{H} CDR2, and a V_{H} CDR3 comprising amino acid sequences comprising 80%, 85%, 90%, 95%, or 99% sequence identity to SEQ ID NOS:26, 27, and 28, respectively.

In some embodiments of various methods, pharmaceutical compositions, kits, uses, or the combinations for use provided herein, the anti-human TIGIT antibody (e.g., monoclonal antibody) or antigen binding fragment thereof comprises a V_{L} region comprising an amino acid sequence comprising 80%, 85%, 90%, 95%, or 99% sequence identity to SEQ ID NO:24, and a V_{H} region comprising an amino acid sequence comprising 80%, 85%, 90%, 95%, or 99% sequence identity to SEQ ID NO:29.

In other embodiments of various methods, pharmaceutical compositions, kits, uses, or the combinations for use provided herein, the anti-human TIGIT antibody (e.g., monoclonal antibody) or antigen binding fragment thereof comprises a light chain comprising or consisting of an amino acid sequence comprising 80%, 85%, 90%, 95%, or 99% sequence identity to SEQ ID NO:25 and a heavy chain comprising or consisting of an amino acid sequence comprising 80%, 85%, 90%, 95%, or 99% sequence identity to SEQ ID NO:30. In one embodiment of various methods, pharmaceutical compositions, kits, or uses provided herein, the anti-human TIGIT monoclonal antibody or antigen binding fragment thereof is BMS-986207 (Bristol-Myers Squibb, New York, N.Y.).

In another embodiment of various methods, pharmaceutical compositions, kits, uses, or the combinations for use provided herein, the anti-human TIGIT monoclonal antibody or antigen binding fragment thereof is OMP-313M32 (OncoMed Pharmaceuticals, Redwood city, CA).

In yet another embodiment of various methods, pharmaceutical compositions, kits, uses, or the combinations for use provided herein, the anti-human TIGIT monoclonal antibody or antigen binding fragment thereof is MTIG7192A (also known as RG6058, U.S. Publ. No. 2017/0088613).

In still another embodiment of various methods, pharmaceutical compositions, kits, uses, or the combinations for use provided herein, the anti-human TIGIT monoclonal antibody or antigen binding fragment thereof is PTZ-201 (Potenza Therapeutics, Cambridge, Mass.; also known as ASP8374, Astellas Pharma, Tokyo, Japan).

In one specific embodiment of various methods, pharmaceutical compositions, kits, uses, or the combinations for use provided herein, the anti-human PD-1 antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human CTLA4 antibody (e.g., monoclonal antibody) or antigen binding fragment thereof comprises a V_{L} CDR1 comprising the amino acid sequence of SEQ ID NO: 14, a V_{L} CDR2 comprising the amino acid sequence of SEQ ID NO: 15, and a V_{L} CDR3 comprising the amino acid sequences as set forth in SEQ ID NOS:16, 17, or 18, and a V_{H} CDR1, a V_{H} CDR2, and a V_{H} CDR3 comprising amino acid sequences as set forth in SEQ ID NOS:11, 12, and 13, respectively; and the anti-human TIGIT antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, uses, or the combinations for use provided herein, the anti-human PD-1 antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; the anti-human CTLA4 antibody (e.g., monoclonal antibody) or antigen binding fragment thereof comprises a V_{L} CDR1, a V_{L} CDR2, and a V_{L} CDR3 comprising the amino acid sequences as set forth in SEQ ID NOS:54, 55, and 56, respectively, and a V_{H} CDR1, a V_{H} CDR2, and a V_{H} CDR3 comprising amino acid sequences as set forth in SEQ ID NOS:57, 105, and 106, respectively; and the anti-human TIGIT antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:4, and a V_{H} region comprising an amino acid sequence as set forth in SEQ ID NO:9; the anti-human CTLA4 antibody (e.g., monoclonal antibody) or antigen binding fragment thereof comprises a V_{L} region comprising an amino acid sequence as set forth in SEQ ID NO:20, and a V_{H} region comprising an amino acid sequence as set forth in SEQ ID NO:19; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:4, and a V_{H} region comprising an amino acid sequence as set forth in SEQ ID NO:9; the anti-human CTLA4 antibody (e.g., monoclonal antibody) or antigen binding fragment thereof comprises a V_{L} region comprising an amino acid sequence as set forth in SEQ ID NO:44, and a V_{H} region comprising an amino acid sequence as set forth in SEQ ID NO:43; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:4, and a V_{H} region comprising an amino acid sequence as set forth in SEQ ID NO:9; the anti-human CTLA4 antibody (e.g., monoclonal antibody) or antigen binding fragment thereof comprises a V_{L} region comprising an amino acid sequence as set forth in SEQ ID NO:42, and a V_{H} region comprising an amino acid sequence as set forth in SEQ ID NO:41; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:4, and a V_{H} region comprising an amino acid sequence as set forth in SEQ ID NO:9; the anti-human CTLA4 antibody (e.g., monoclonal antibody) or antigen binding fragment thereof comprises a V_{L} region comprising an amino acid sequence as set forth in SEQ ID NO:44, and a V_{H} region comprising an amino acid sequence as set forth in SEQ ID NO:43; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:4, and a V_{H} region comprising an amino acid sequence as set forth in SEQ ID NO:9; the anti-human CTLA4 antibody (e.g., monoclonal antibody) or antigen binding fragment thereof comprises a V_{L} region comprising an amino acid sequence as set forth in SEQ ID NO:47, and a V_{H} region comprising an amino acid sequence as set forth in SEQ ID NO:46; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:4, and a V_{H} region comprising an amino acid sequence as set forth in SEQ ID NO:9; the anti-human CTLA4 antibody (e.g., monoclonal antibody) or antigen binding fragment thereof comprises a V_{L} region comprising an amino acid sequence as set forth in SEQ ID NO:49, and a V_{H} region comprising an amino acid sequence as set forth in SEQ ID NO:48; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:4, and a V_{H} region comprising an amino acid sequence as set forth in SEQ ID NO:9; the anti-human CTLA4 antibody (e.g., monoclonal antibody) or antigen binding fragment thereof comprises a V_{L} region comprising an amino acid sequence as set forth in SEQ ID NO:51, and a V_{H} region comprising an amino acid sequence as set forth in SEQ ID NO:50; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:4, and a V_{H} region comprising an amino acid sequence as set forth in SEQ ID NO:9; the anti-human CTLA4 antibody (e.g., monoclonal antibody) or antigen binding fragment thereof comprises a V_{L} region comprising an amino acid sequence as set forth in SEQ ID NO:108, and a V_{H} region comprising an amino acid sequence as set forth in SEQ ID NO:107; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In yet another specific embodiment of various methods, pharmaceutical compositions, kits, uses, or the combinations for use provided herein, the anti-human PD-1 antibody (e.g., monoclonal antibody) or antigen binding fragment thereof comprises a light chain comprising or consisting of an amino acid sequence as set forth in SEQ ID NO:5 and a heavy chain comprising or consisting of an amino acid sequence as set forth in SEQ ID NO:10; the anti-human CTLA4 antibody (e.g., monoclonal antibody) or antigen binding fragment thereof comprises a light chain comprising or consisting of an amino acid sequence as set forth in SEQ ID NO:53 and a heavy chain comprising or consisting of an amino acid sequence as set forth in SEQ ID NO:52; and the anti-human TIGIT antibody (e.g., monoclonal antibody) or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In yet another specific embodiment of various methods, pharmaceutical compositions, kits, uses, or the combinations for use provided herein, the anti-human PD-1 antibody (e.g., monoclonal antibody) or antigen binding fragment thereof comprises a light chain comprising or consisting of an amino acid sequence as set forth in SEQ ID NO:5 and a heavy chain comprising or consisting of an amino acid sequence as set forth in SEQ ID NO:10; the anti-human CTLA4 antibody (e.g., monoclonal antibody) or antigen binding fragment thereof comprises a light chain comprising or consisting of an amino acid sequence as set forth in SEQ ID NO:110 and a heavy chain comprising or consisting of an amino acid sequence as set forth in SEQ ID NO:109; and the anti-human TIGIT antibody (e.g., monoclonal antibody) or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In one specific embodiment of various methods, pharmaceutical compositions, kits, uses, or the combinations for use provided herein, the anti-human PD-1 antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human CTLA4 antibody (e.g., monoclonal antibody) or antigen binding fragment thereof comprises a V_{L} CDR1 comprising the amino acid sequence of SEQ ID NO: 14, a V_{L} CDR2 comprising the amino acid sequence of SEQ ID NO: 15, and a V_{L} CDR3 comprising the amino acid sequences as set forth in SEQ ID NOS:16, 17, or 18, and a V_{H} CDR1, a V_{H} CDR2, and a V_{H} CDR3 comprising amino acid sequences as set forth in SEQ ID NOS:11, 12, and 13, respectively; and the anti-human TIGIT antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment of various methods, pharmaceutical compositions, kits, uses, or the combinations for use provided herein, the anti-human PD-1 antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; the anti-human CTLA4 antibody (e.g., monoclonal antibody) or antigen binding fragment thereof comprises a V_{L} CDR1, a V_{L} CDR2, and a V_{L} CDR3 comprising the amino acid sequences as set forth in SEQ ID NOS:54, 55, and 56, respectively, and a V_{H} CDR1, a V_{H} CDR2, and a V_{H} CDR3 comprising amino acid sequences as set forth in SEQ ID NOS:57, 105, and 106, respectively; and the anti-human TIGIT antibody (e.g., monoclonal antibody) or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:34, and a V_{H} region comprising an amino acid sequence as set forth in SEQ ID NO:39; the anti-human CTLA4 antibody (e.g., monoclonal antibody) or antigen binding fragment thereof comprises a V_{L} region comprising an amino acid sequence as set forth in SEQ ID NO:20, and a V_{H} region comprising an amino acid sequence as set forth in SEQ ID NO:19; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:34, and a V_{H} region comprising an amino acid sequence as set forth in SEQ ID NO:39; the anti-human CTLA4 antibody (e.g., monoclonal antibody) or antigen binding fragment thereof comprises a V_{L} region comprising an amino acid sequence as set forth in SEQ ID NO:42, and a V_{H} region comprising an amino acid sequence as set forth in SEQ ID NO:41; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:34, and a V_{H} region comprising an amino acid sequence as set forth in SEQ ID NO:39; the anti-human CTLA4 antibody (e.g., monoclonal antibody) or antigen binding fragment thereof comprises a V_{L} region comprising an amino acid sequence as set forth in SEQ ID NO:44, and a V_{H} region comprising an amino acid sequence as set forth in SEQ ID NO:43; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:34, and a V_{H} region comprising an amino acid sequence as set forth in SEQ ID NO:39; the anti-human CTLA4 antibody (e.g., monoclonal antibody) or antigen binding fragment thereof comprises a V_{L} region comprising an amino acid sequence as set forth in SEQ ID NO:44, and a V_{H} region comprising an amino acid sequence as set forth in SEQ ID NO:45; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:34, and a V_{H} region comprising an amino acid sequence as set forth in SEQ ID NO:39; the anti-human CTLA4 antibody (e.g., monoclonal antibody) or antigen binding fragment thereof comprises a V_{L} region comprising an amino acid sequence as set forth in SEQ ID NO:47, and a V_{H} region comprising an amino acid sequence as set forth in SEQ ID NO:46; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:34, and a V_{H} region comprising an amino acid sequence as set forth in SEQ ID NO:39; the anti-human CTLA4 antibody (e.g., monoclonal antibody) or antigen binding fragment thereof comprises a V_{L} region comprising an amino acid sequence as set forth in SEQ ID NO:49, and a V_{H} region comprising an amino acid sequence as set forth in SEQ ID NO:48; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:34, and a V_{H} region comprising an amino acid sequence as set forth in SEQ ID NO:39; the anti-human CTLA4 antibody (e.g., monoclonal antibody) or antigen binding fragment thereof comprises a V_{L} region comprising an amino acid sequence as set forth in SEQ ID NO:51, and a V_{H} region comprising an amino acid sequence as set forth in SEQ ID NO:50; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In another specific embodiment of various methods, pharmaceutical compositions, kits, uses, or the combinations for use provided herein, the anti-human PD-1 antibody (e.g., monoclonal antibody) or antigen binding fragment thereof comprises a V_{L} region comprising an amino acid sequence as set forth in SEQ ID NO:34, and a V_{H} region comprising an amino acid sequence as set forth in SEQ ID NO:39; the anti-human CTLA4 antibody (e.g., monoclonal antibody) or antigen binding fragment thereof comprises a V_{L} region comprising an amino acid sequence as set forth in SEQ ID NO:108, and a V_{H} region comprising an amino acid sequence as set forth in SEQ ID NO:107; and the anti-human TIGIT antibody (e.g., monoclonal antibody) or antigen binding fragment thereof comprises a V_{L} region comprising an amino acid sequence as set forth in SEQ ID NO:24, and a V_{H} region comprising an amino acid sequence as set forth in SEQ ID NO:29.

In yet another specific embodiment of various methods, pharmaceutical compositions, kits, uses, or the combinations for use provided herein, the anti-human PD-1 antibody (e.g., monoclonal antibody) or antigen binding fragment thereof comprises a light chain comprising or consisting of an amino acid sequence as set forth in SEQ ID NO:35 and a heavy chain comprising or consisting of an amino acid sequence as set forth in SEQ ID NO:40; the anti-human CTLA4 antibody (e.g., monoclonal antibody) or antigen binding fragment thereof comprises a light chain comprising or consisting of an amino acid sequence as set forth in SEQ ID NO:53 and a heavy chain comprising or consisting of an amino acid sequence as set forth in SEQ ID NO:52; and the anti-human TIGIT antibody (e.g., monoclonal antibody) or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In yet another specific embodiment of various methods, pharmaceutical compositions, kits, uses, or the combinations for use provided herein, the anti-human PD-1 antibody (e.g., monoclonal antibody) or antigen binding fragment thereof comprises a light chain comprising or consisting of an amino acid sequence as set forth in SEQ ID NO:5 and a heavy chain comprising or consisting of an amino acid sequence as set forth in SEQ ID NO:10; the anti-human CTLA4 antibody (e.g., monoclonal antibody) or antigen binding fragment thereof comprises a light chain comprising or consisting of an amino acid sequence as set forth in SEQ ID NO:53 and a heavy chain comprising or consisting of an amino acid sequence as set forth in SEQ ID NO:52; and the anti-human TIGIT antibody (e.g., monoclonal antibody) or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In some embodiments of various methods, pharmaceutical compositions, kits, or the combinations of use provided herein, the anti-human CTLA4 antibody or antigen binding fragment thereof is administered every six weeks.

In yet another specific embodiment of various methods, pharmaceutical compositions, kits, uses, or the combinations for use provided herein, the anti-human PD-1 monoclonal antibody is pembrolizumab, and 200 mg pembrolizumab is administered once every three weeks.

In yet another specific embodiment of various methods, pharmaceutical compositions, kits, uses, or the combinations for use provided herein, the anti-human PD-1 monoclonal antibody is pembrolizumab, wherein and the subject (e.g., human patient) is administered 400 mg pembrolizumab, and wherein pembrolizumab is administered once every six weeks.

In yet another specific embodiment of various methods, pharmaceutical compositions, kits, uses, or the combinations for use provided herein, 200 mg of the anti-human TIGIT monoclonal antibody or antigen binding fragment thereof is administered.

In yet another specific embodiment of various methods, pharmaceutical compositions, kits, uses, or the combinations for use provided herein, 25 mg of the anti-human CTLA4 monoclonal antibody or antigen binding fragment thereof is administered.

In yet another specific embodiment of various methods, pharmaceutical compositions, kits, uses, or the combinations for use provided herein, the anti-human TIGIT monoclonal antibody or antigen binding fragment thereof is administered every three weeks.

In yet another specific embodiment of various methods, pharmaceutical compositions, kits, uses, or the combinations for use provided herein, the anti-human CTLA4 monoclonal antibody or antigen binding fragment thereof is administered every six weeks.

In yet another specific embodiment of various methods, pharmaceutical compositions, kits, uses, or the combinations for use provided herein, the anti-human TIGIT monoclonal antibody or antigen binding fragment thereof is administered every three weeks.

In yet another specific embodiment of various methods, pharmaceutical compositions, kits, uses, or the combinations for use provided herein, 25 mg of the anti-human CTLA4 monoclonal antibody or antigen binding fragment thereof is administered every six weeks.

In one specific embodiment of various methods, pharmaceutical compositions, kits, uses, or the combinations for use provided herein, the anti-human PD-1 monoclonal antibody or antigen binding fragment thereof is cemiplimab.

In one specific embodiment of various methods, pharmaceutical compositions, kits, uses, or the combinations for use provided herein, the anti-human PD-1 monoclonal antibody or antigen binding fragment thereof is pidilizumab.

In one specific embodiment of various methods, pharmaceutical compositions, kits, uses, or the combinations for use provided herein, the anti-human PD-1 monoclonal antibody or antigen binding fragment thereof is AMP-514.

In one specific embodiment of various methods, pharmaceutical compositions, kits, uses, or the combinations for use provided herein, the anti-human PD-1 monoclonal antibody or antigen binding fragment thereof is PDR001.

In one specific embodiment of various methods, pharmaceutical compositions, kits, uses, or the combinations for use provided herein, the anti-human PD-1 monoclonal antibody or antigen binding fragment thereof is BGB-A317.

In one specific embodiment of various methods, pharmaceutical compositions, kits, uses, or the combinations for use provided herein, the anti-human PD-1 monoclonal antibody or antigen binding fragment thereof is MGA012.

In one specific embodiment of various methods, pharmaceutical compositions, kits, uses, or the combinations for use provided herein, the anti-human TIGIT monoclonal antibody or antigen binding fragment thereof is BMS-986207.

In one specific embodiment of various methods, pharmaceutical compositions, kits, uses, or the combinations for use provided herein, the anti-human TIGIT monoclonal antibody or antigen binding fragment thereof is OMP-313M32.

In one specific embodiment of various methods, pharmaceutical compositions, kits, uses, or the combinations for use provided herein, the anti-human TIGIT monoclonal antibody or antigen binding fragment thereof is MTIG7192A (RG6058).

In one specific embodiment of various methods, pharmaceutical compositions, kits, uses, or the combinations for use provided herein, the anti-human TIGIT monoclonal antibody or antigen binding fragment thereof is PTZ-201 (ASP8374).

In one specific embodiment of various methods, pharmaceutical compositions, kits, uses, or the combinations for use provided herein, the anti-human TIGIT monoclonal antibody or antigen binding fragment thereof is BMS-986207.

In one specific embodiment of various methods, pharmaceutical compositions, kits, uses, or the combinations for use provided herein, the anti-human TIGIT monoclonal antibody or antigen binding fragment thereof is OMP-313M32.

In one specific embodiment of various methods, pharmaceutical compositions, kits, uses, or the combinations for use provided herein, anti-human TIGIT monoclonal antibody or antigen binding fragment thereof is MTIG7192A (RG6058).

In one specific embodiment of various methods, pharmaceutical compositions, kits, uses, or the combinations for use provided herein, the anti-human TIGIT monoclonal antibody or antigen binding fragment thereof is PTZ-201 (ASP8374).

In certain embodiments, the subject (e.g., a human patient) is administered:
(a) 200 mg or 2 mg/kg pembrolizumab or an antigen binding fragment thereof;
(b) an anti-human CTLA4 antibody or antigen binding fragment thereof, and
(c) an anti-human TIGIT antibody or antigen binding fragment thereof, wherein pembrolizumab is administered once every three weeks.

In certain embodiments, the subject (e.g., a human patient) is administered:
(a) 200 mg pembrolizumab or an antigen binding fragment;
(b) an anti-human CTLA4 antibody or antigen binding fragment thereof, and
(c) an anti-human TIGIT antibody or antigen binding fragment thereof, wherein pembrolizumab is administered once every three weeks.

In other embodiments, the subject (e.g., a human patient) is administered:
(a) 2 mg/kg pembrolizumab or an antigen binding fragment thereof,
(b) an anti-human CTLA4 antibody or antigen binding fragment thereof, and
(c) an anti-human TIGIT antibody or antigen binding fragment thereof, wherein pembrolizumab is administered once every three weeks.

In certain embodiments, the subject (e.g., a human patient) is administered:
(a) 400 mg pembrolizumab or an antigen binding fragment;
(b) an anti-human CTLA4 antibody or antigen binding fragment thereof, and
(c) an anti-human TIGIT antibody or antigen binding fragment thereof, wherein pembrolizumab is administered once every six weeks.

In some embodiments, the subject (e.g., a human patient) is administered:
(a) 240 mg or 3 mg/kg nivolumab or an antigen binding fragment thereof,
(b) an anti-human CTLA4 antibody or antigen binding fragment thereof, and
(c) an anti-human TIGIT antibody or antigen binding fragment thereof, wherein nivolumab is administered once every two weeks.

In some embodiments, the subject (e.g., a human patient) is administered:
(a) 240 mg nivolumab or an antigen binding fragment thereof,
(b) an anti-human CTLA4 antibody or antigen binding fragment thereof, and
(c) an anti-human TIGIT antibody or antigen binding fragment thereof, wherein nivolumab is administered once every two weeks.

In some embodiments, the subject (e.g., a human patient) is administered:
(a) 3 mg/kg nivolumab or an antigen binding fragment thereof,
(b) an anti-human CTLA4 antibody or antigen binding fragment thereof, and
(c) an anti-human TIGIT antibody or antigen binding fragment thereof, wherein nivolumab is administered once every two weeks.

In some embodiments, the subject (e.g., a human patient) is administered:
(a) 350 mg cemiplimab or an antigen binding fragment thereof,
(b) an anti-human CTLA4 antibody or antigen binding fragment thereof, and
(c) an anti-human TIGIT antibody or antigen binding fragment thereof, wherein nivolumab is administered once every three weeks.

In one specific embodiment of various methods, pharmaceutical compositions, kits, uses, or the combinations for use provided herein, 200 mg of the anti-human TIGIT antibody (e.g., monoclonal antibody) or antigen binding fragment thereof is administered.

In one specific embodiment of various methods, pharmaceutical compositions, kits, uses, or the combinations for use provided herein, 25 mg of the anti-human CTLA4 antibody (e.g., monoclonal antibody) or antigen binding fragment thereof is administered.

In one specific embodiment of various methods, pharmaceutical compositions, kits, uses, or the combinations for use provided herein, the anti-human TIGIT antibody (e.g., monoclonal antibody) or antigen binding fragment thereof is administered every three weeks.

In one specific embodiment of various methods, pharmaceutical compositions, kits, uses, or the combinations for use provided herein, anti-human CTLA4 antibody (e.g., monoclonal antibody) or antigen binding fragment thereof is administered every six weeks.

In one specific embodiment of various methods, pharmaceutical compositions, kits, uses, or the combinations for use provided herein, 200 mg of the anti-human TIGIT antibody (e.g., monoclonal antibody) or antigen binding fragment thereof is administered every three weeks.

In one specific embodiment of various methods, pharmaceutical compositions, kits, uses, or the combinations for use provided herein, 25 mg of the anti-human CTLA4 antibody (e.g., monoclonal antibody) or antigen binding fragment thereof is administered every six weeks.

In various embodiments of the method, the anti-PD-1 antibody (e.g., monoclonal antibody) or antigen binding fragment is administered via intravenous infusion.

In various embodiments of the method, the anti-CTLA4 antibody (e.g., monoclonal antibody) or antigen binding fragment is administered via intravenous infusion.

In various embodiments of the method, the anti-TIGIT antibody (e.g., monoclonal antibody) or antigen binding fragment is administered via intravenous infusion.

In one specific embodiment of various methods, pharmaceutical compositions, kits, uses, or the combinations for use provided herein, the subject (e.g., a human patient) is administered:
(a) 200 mg, 400 mg, or 2 mg/kg pembrolizumab or an antigen binding fragment thereof;
(b) an anti-human CTLA4 antibody or antigen binding fragment thereof comprising a V_{L} region comprising an amino acid sequence as set forth in SEQ ID NO:44, and a V_{H} region comprising an amino acid sequence as set forth in SEQ ID NO:45; and
(c) an anti-human TIGIT antibody or antigen binding fragment thereof comprising a V_{L} region comprising an amino acid sequence as set forth in SEQ ID NO:24, and a V_{H} region comprising an amino acid sequence as set forth in SEQ ID NO:29.

In one specific embodiment of various methods, pharmaceutical compositions, kits, uses, or the combinations for use provided herein, the subject (e.g., a human patient) is administered
(a) 200 mg, 400 mg, or 2 mg/kg pembrolizumab or an antigen binding fragment thereof;
(b) an anti-human CTLA4 antibody or antigen binding fragment thereof consisting of a V_{L} region comprising an amino acid sequence as set forth in SEQ ID NO:44, and a V_{H} region comprising an amino acid sequence as set forth in SEQ ID NO:45; and
(c) an anti-human TIGIT antibody or antigen binding fragment thereof consisting of a V_{L} region comprising an amino acid sequence as set forth in SEQ ID NO:24, and a V_{H} region comprising an amino acid sequence as set forth in SEQ ID NO:29.

In various embodiments, the anti-PD-1 antibody (e.g., monoclonal antibody) or antigen binding fragment is administered via intravenous infusion.

In various embodiments, the anti-CTLA4 antibody (e.g., monoclonal antibody) or antigen binding fragment is administered via intravenous infusion.

In various embodiments, the anti-TIGIT antibody (e.g., monoclonal antibody) or antigen binding fragment is administered via intravenous infusion.

In one specific embodiment of various methods, pharmaceutical compositions, kits, uses, or the combinations for use provided herein, 200 mg of pembrolizumab is administered every three weeks.

In one specific embodiment of various methods, pharmaceutical compositions, kits, uses, or the combinations for use provided herein, 400 mg of pembrolizumab is administered every six weeks.

In one specific embodiment of various methods, pharmaceutical compositions, kits, uses, or the combinations for use provided herein, 200 mg of the anti-human TIGIT antibody (e.g., monoclonal antibody) or antigen binding fragment thereof is administered every three weeks.

In one specific embodiment of various methods, pharmaceutical compositions, kits, uses, or the combinations for use provided herein, 25 mg of the anti-human CTLA4 antibody (e.g., monoclonal antibody) or antigen binding fragment thereof is administered every six weeks.

In one specific embodiment of various methods, pharmaceutical compositions, kits, uses, or the combinations for use provided herein, the subject (e.g., a human patient) is administered:
(a) 200 mg, 400 mg, or 2 mg/kg pembrolizumab or an antigen binding fragment thereof;
(b) an anti-human CTLA4 antibody or antigen binding fragment thereof comprising an amino acid sequence as set forth in SEQ ID NO:53 and a heavy chain comprising an amino acid sequence as set forth in SEQ ID NO:52; and
(c) an anti-human TIGIT antibody or antigen binding fragment thereof comprising an amino acid sequence as set forth in SEQ ID NO:25 and a heavy chain comprising an amino acid sequence as set forth in SEQ ID NO:30.

In one specific embodiment of various methods, pharmaceutical compositions, kits, uses, or the combinations for use provided herein, the subject (e.g., a human patient) is administered
(a) 200 mg, 400 mg, or 2 mg/kg pembrolizumab or an antigen binding fragment thereof;
(b) an anti-human CTLA4 antibody or antigen binding fragment thereof consisting of an amino acid sequence as set forth in SEQ ID NO:53 and a heavy chain comprising an amino acid sequence as set forth in SEQ ID NO:52; and
(c) an anti-human TIGIT antibody or antigen binding fragment thereof consisting of an amino acid sequence as set forth in SEQ ID NO:25 and a heavy chain comprising an amino acid sequence as set forth in SEQ ID NO:30.

In various embodiments, the anti-PD-1 antibody (e.g., monoclonal antibody) or antigen binding fragment is administered via intravenous infusion.

In various embodiments, the anti-CTLA4 antibody (e.g., monoclonal antibody) or antigen binding fragment is administered via intravenous infusion.

In various embodiments, the anti-TIGIT antibody (e.g., monoclonal antibody) or antigen binding fragment is administered via intravenous infusion.

In various embodiments, 200 mg of pembrolizumab is administered every three weeks.

In various embodiments, 400 mg of pembrolizumab is administered every six weeks.

In various embodiments, 200 mg of the anti-human TIGIT antibody (e.g., monoclonal antibody) or antigen binding fragment thereof is administered every three weeks.

In various embodiments, 25 mg of the anti-human CTLA4 antibody (e.g., monoclonal antibody) or antigen binding fragment thereof is administered every six weeks.

In various embodiments, the methods comprise treating cancer.

In various embodiments, the cancer comprises at least one tumor.

In various embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In various embodiments, the cancer is metastatic. In various embodiments, the cancer is relapsed. In various embodiments, the cancer is refractory. For example, the refractory cancer is any of the cancers described herein.

In one aspect, provided herein is a method of treating a cancer comprising administering to a subject in need thereof:
(a) 200 mg, 400 mg, or 2 mg/kg pembrolizumab or antigen binding fragment thereof;
(b) an effective amount of an anti-human CTLA4 antibody or antigen binding fragment thereof comprising a V_{L} region comprising an amino acid sequence as set forth in SEQ ID NO:44, and a V_{H} region comprising an amino acid sequence as set forth in SEQ ID NO:45; and
(c) an effective amount of an anti-human TIGIT antibody or antigen binding fragment thereof comprising a V_{L} region comprising an amino acid sequence as set forth in SEQ ID NO:24, and a V_{H} region comprising an amino acid sequence as set forth in SEQ ID NO:29.

In one aspect, provided herein is a method of treating a cancer comprising administering to a subject in need thereof
(a) 200 mg, 400 mg, or 2 mg/kg pembrolizumab or antigen binding fragment thereof;
(b) an effective amount of an anti-human CTLA4 antibody or antigen binding fragment thereof comprising a V_{L} region consisting of an amino acid sequence as set forth in SEQ ID NO:44, and a V_{H} region consisting of an amino acid sequence as set forth in SEQ ID NO:45; and
(c) an effective amount of an anti-human TIGIT antibody or antigen binding fragment thereof comprising a V_{L} region consisting of an amino acid sequence as set forth in SEQ ID NO:24, and a V_{H} region consisting of an amino acid sequence as set forth in SEQ ID NO:29.

In certain embodiments, the pembrolizumab or antigen binding fragment thereof, the anti-CTLA4 antibody or antigen binding fragment thereof, and/or the anti-TIGIT antibody or antigen binding fragment thereof is administered via intravenous infusion.

In certain embodiments, the anti-CTLA4 antibody is a monoclonal antibody.

In certain embodiments, the anti-TIGIT antibody is a monoclonal antibody.

In certain embodiments, 200 mg of pembrolizumab is administered every three weeks.

In certain embodiments, 400 mg of pembrolizumab is administered every six weeks.

In certain embodiments, 200 mg of the anti-human TIGIT antibody or antigen binding fragment thereof is administered every three weeks.

In certain embodiments, 25 mg of the anti-human CTLA4 antibody or antigen binding fragment thereof is administered every six weeks.

In one aspect, provided herein is a method of treating a cancer comprising administering to a subject in need thereof:
(a) 200 mg, 400 mg, or 2 mg/kg pembrolizumab or antigen binding fragment thereof;
(b) an effective amount of an anti-human CTLA4 antibody or antigen binding fragment thereof comprising a light chain comprising an amino acid sequence as set forth in SEQ ID NO:53 and a heavy chain comprising an amino acid sequence as set forth in SEQ ID NO:52; and
(c) an effective amount of an anti-human TIGIT antibody or antigen binding fragment thereof comprising a light chain comprising an amino acid sequence as set forth in SEQ ID NO:25 and a heavy chain comprising an amino acid sequence as set forth in SEQ ID NO:30.

In one aspect, provided herein is a method of treating a cancer comprising administering to a subject in need thereof:
(a) 200 mg, 400 mg, or 2 mg/kg pembrolizumab or antigen binding fragment thereof;
(b) an effective amount of an anti-human CTLA4 antibody or antigen binding fragment thereof consisting of a light chain comprising an amino acid sequence as set forth in SEQ ID NO:53 and a heavy chain comprising an amino acid sequence as set forth in SEQ ID NO:52; and
(c) an effective amount an anti-human TIGIT antibody or antigen binding fragment thereof consisting of a light chain comprising an amino acid sequence as set forth in SEQ ID NO:25 and a heavy chain comprising an amino acid sequence as set forth in SEQ ID NO:30.

In certain embodiments, the pembrolizumab or antigen binding fragment thereof, the anti-CTLA4 antibody or antigen binding fragment thereof, and/or the anti-TIGIT antibody or antigen binding fragment thereof is administered via intravenous infusion.

In certain embodiments, the anti-CTLA4 antibody is a monoclonal antibody.

In certain embodiments, the anti-TIGIT antibody is a monoclonal antibody.

In certain embodiments, 200 mg of pembrolizumab is administered every three weeks.

In certain embodiments, 400 mg of pembrolizumab is administered every six weeks.

In certain embodiments, 200 mg of the anti-human TIGIT antibody or antigen binding fragment thereof is administered every three weeks.

In certain embodiments, 25 mg of the anti-human CTLA4 antibody or antigen binding fragment thereof is administered every six weeks.

In certain embodiments, the method is for treating cancer.

In certain embodiments, the cancer comprises at least one tumor.

In certain embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic.

In certain embodiments, the cancer is relapsed.

In certain embodiments, the cancer is refractory.

In certain embodiments, the refractory cancer (e.g., melanoma) has progressed on treatment with an anti-PD-1/L1 mAb administered either as monotherapy, or in combination with a checkpoint inhibitor or other therapy.

In certain embodiments, the subject has received at least 2 doses of an approved anti-PD-1/L1 monoclonal antibody.

In certain embodiments, the subject has demonstrated disease progression after PD-1/L1 treatment as defined by RECIST 1.1.

In certain embodiments, the anti-PD-1 antibody or antigen binding fragment, the anti-TIGIT antibody or antigen binding fragment, and the anti-CTLA4 antibody or antigen binding fragment are administered contemporaneously.

In certain embodiments, the order of administration is the anti-PD-1 antibody followed by the anti-TIGIT antibody and then the anti-CTLA4 antibody.

In certain embodiments, the subject has at least one melanoma selected from the group consisting of: PD-1 refractory melanoma, first-line advanced melanoma, and neoadjuvant melanoma. In various embodiments, the cancer is locally advanced or resectable. In various embodiments, the cancer the cancer has been treated or is being treated with an adjuvant. In various embodiments, the cancer has been treated or is being treated with a neoadjuvant.

In certain embodiments, the method, pharmaceutical composition, kit, or combinations for use further comprises at least one therapeutic agent and/or active agent. For example, the therapeutic agent and/or active agent comprises an antibody or fragment thereof, an immunomodulator, a hormone, a cytotoxic agent, an enzyme, a radionuclide, a second antibody conjugated to at least one immunomodulator, enzyme, radioactive label, hormone, antisense oligonucleotide, biotherapeutic agent, chemotherapeutic agent, cytotoxic agent, or a combination thereof.

### BRIEF DESCRIPTION OF THE DRAWINGS

FIGS. 1A-1G are a series of graphs showing tumor volume (mm³) and progression as a function of time (days) for murine subjects in an EMT6 tumor model treated with murine and/or rat-mouse chimeric antibodies. Subjects in the EMT6 (150 mm³) tumor model were treated with either: vehicle (A); rat-mouse chimeric anti-TIGIT antibody 18G10-IgG2a (B); anti-mouse CTLA4 antibody (C); anti-mouse PD-1 monoclonal antibody (muDX400) (D); a combination of rat-mouse chimeric anti-TIGIT antibody 18G10-IgG2a and anti-mouse CTLA4 antibody (E); a combination of rat-mouse chimeric anti-TIGIT antibody 18G10-IgG2a and anti-mouse PD-1 monoclonal antibody muDX400 (F); and a combination of rat-mouse chimeric anti-TIGIT antibody 18G10-IgG2a, anti-CTLA4 antibody anti-mouse PD-1 monoclonal antibody muDX400.

FIG. 2A and FIG. 2B are two graphs showing tumor volume (mm³) and progression as a function of time (days) for murine subjects in an EMT6 tumor model treated with murine and/or rat-mouse chimeric antibodies binding to either PD-1, TIGIT, or CTLA4.

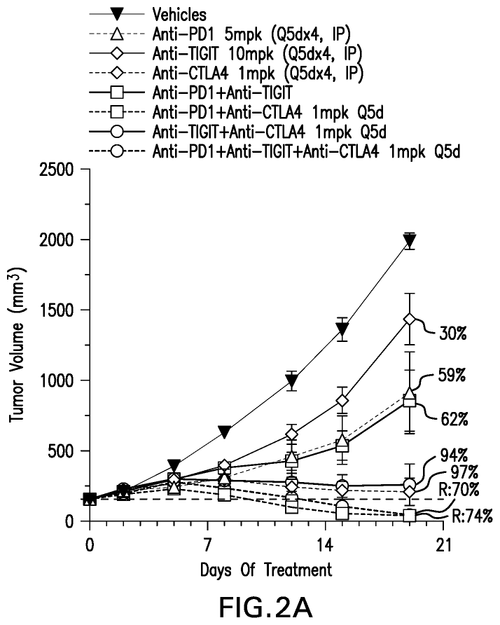

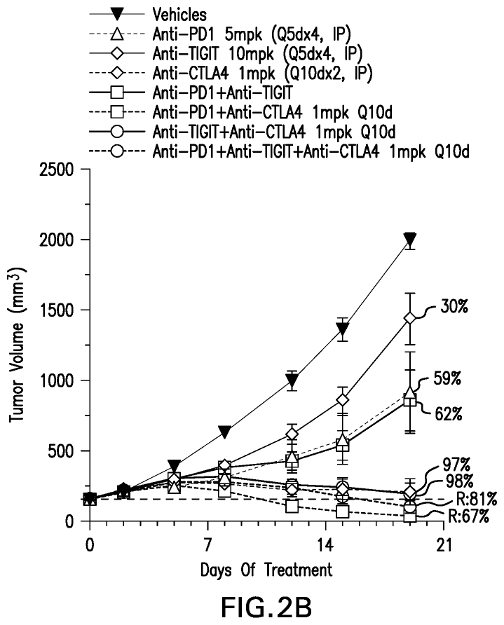

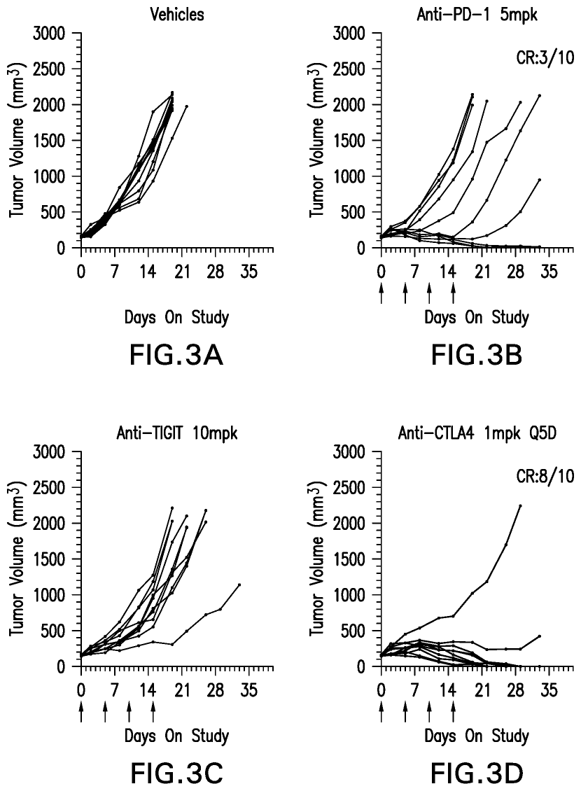

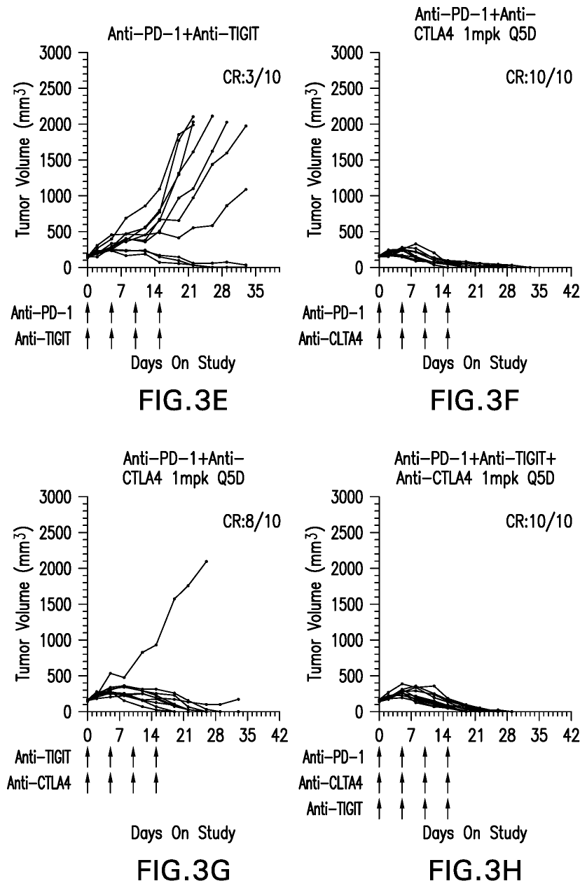

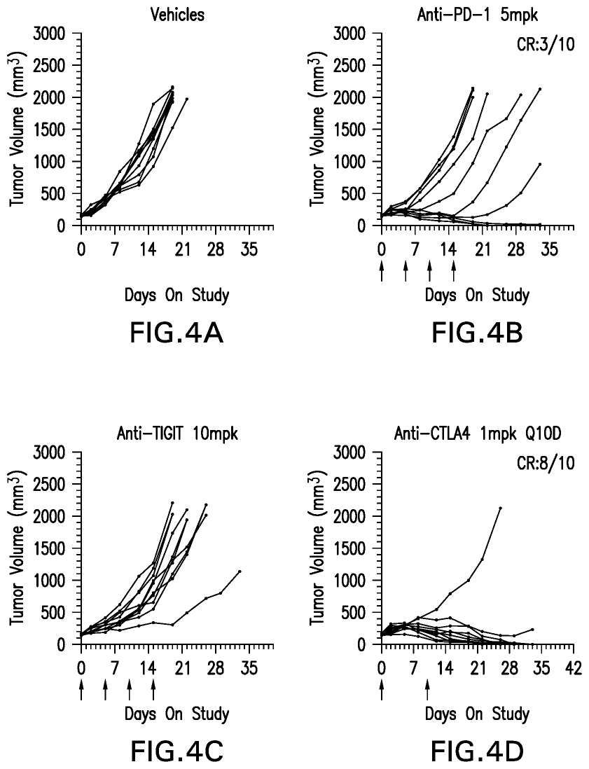

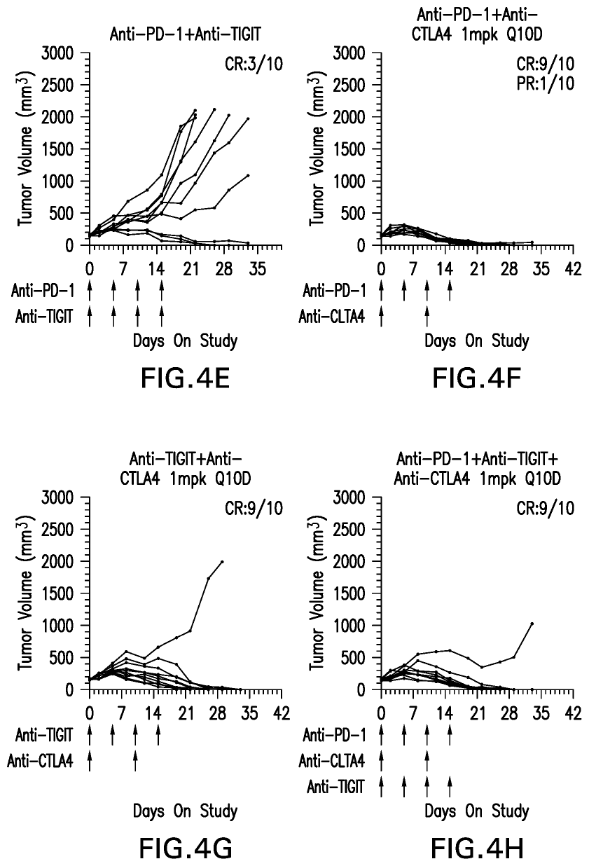

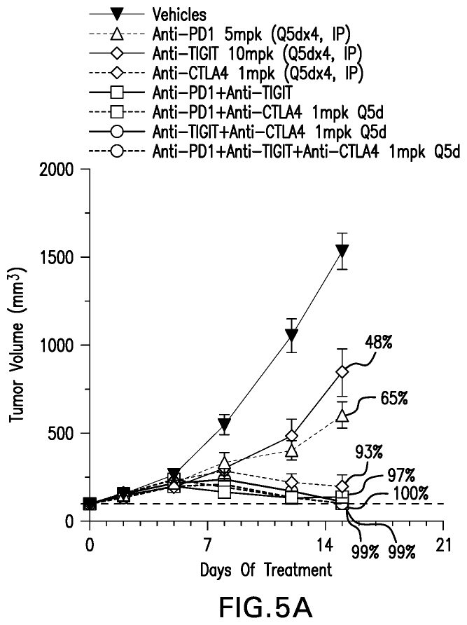

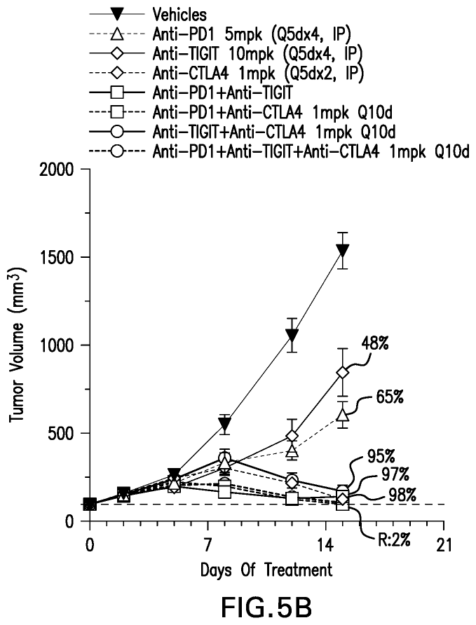

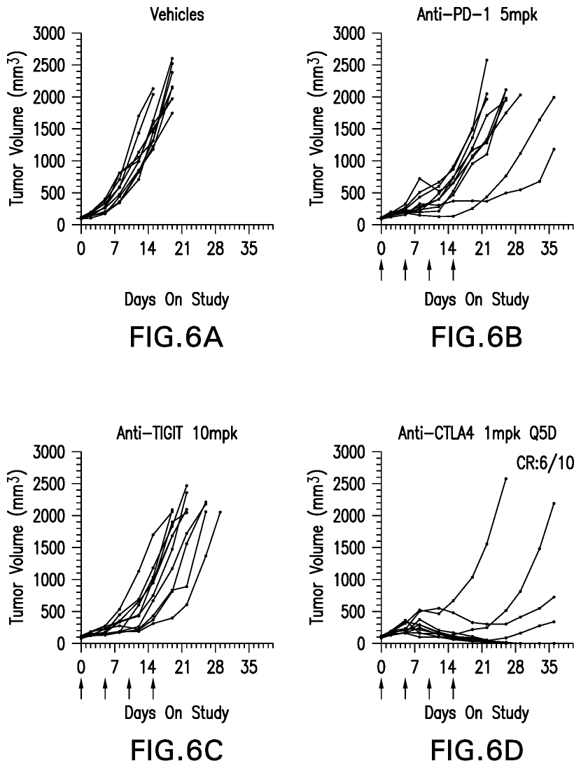

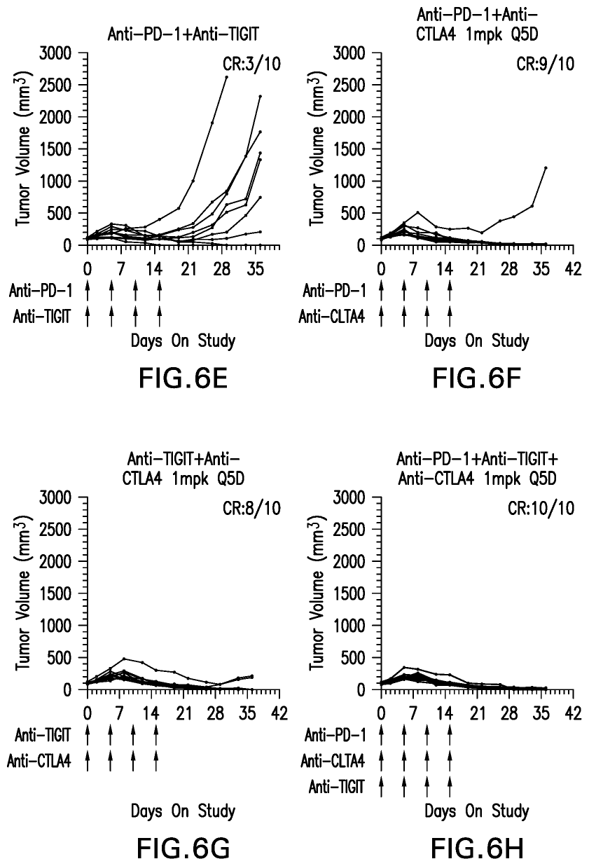

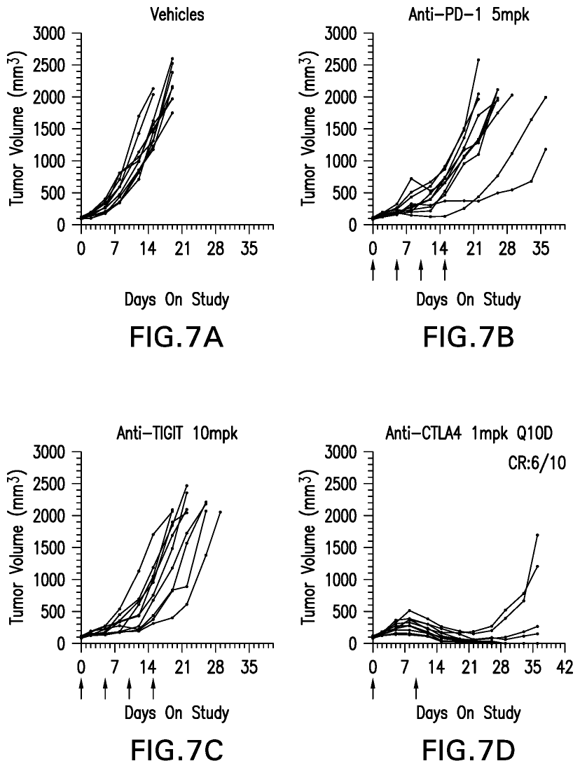

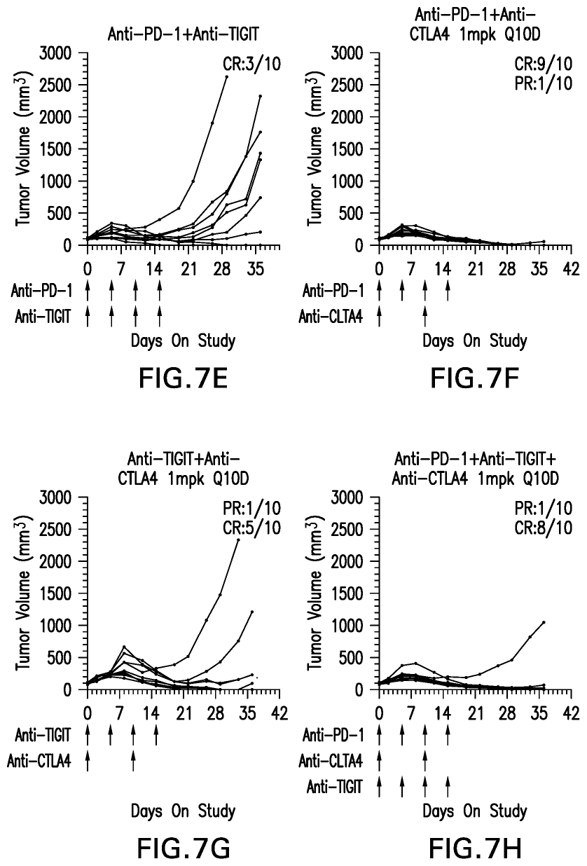

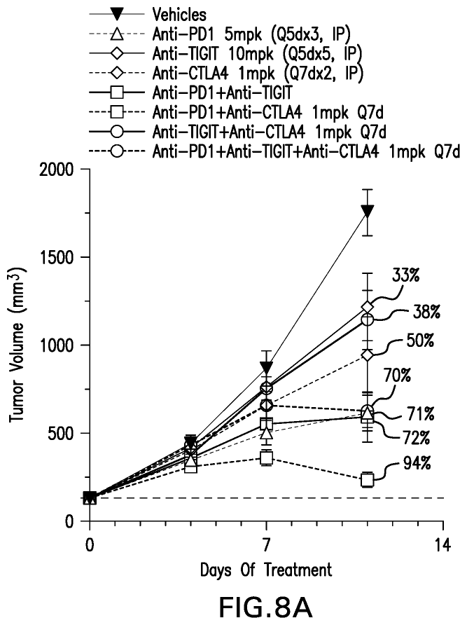

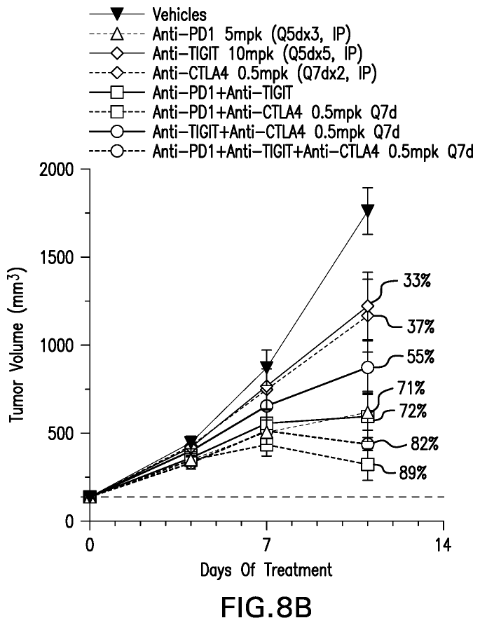

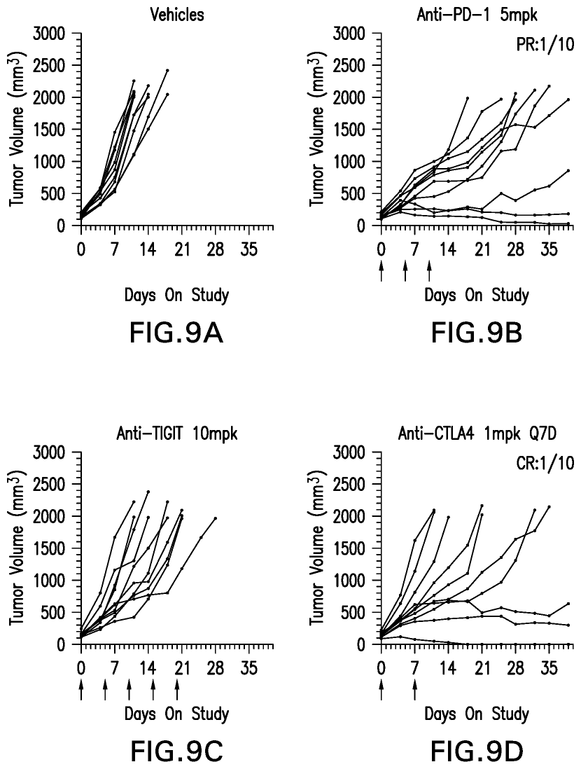

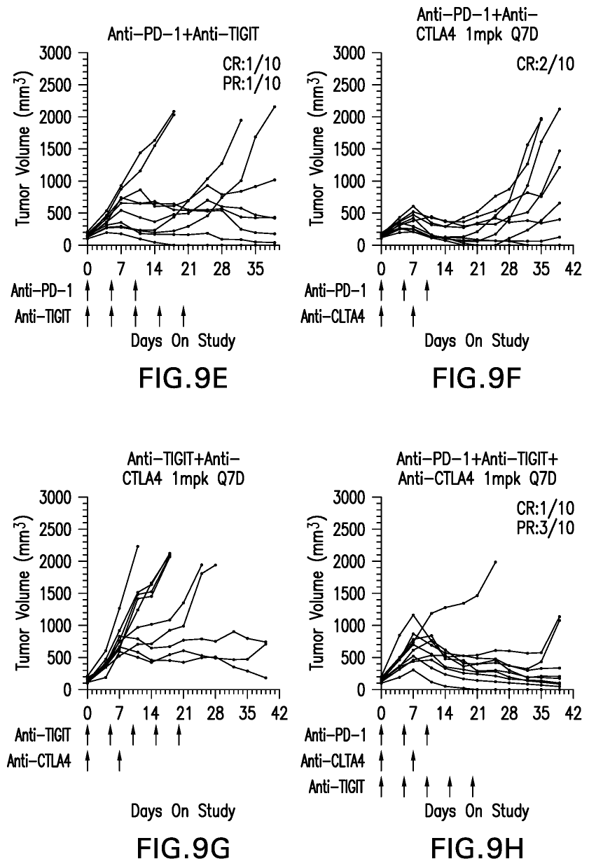

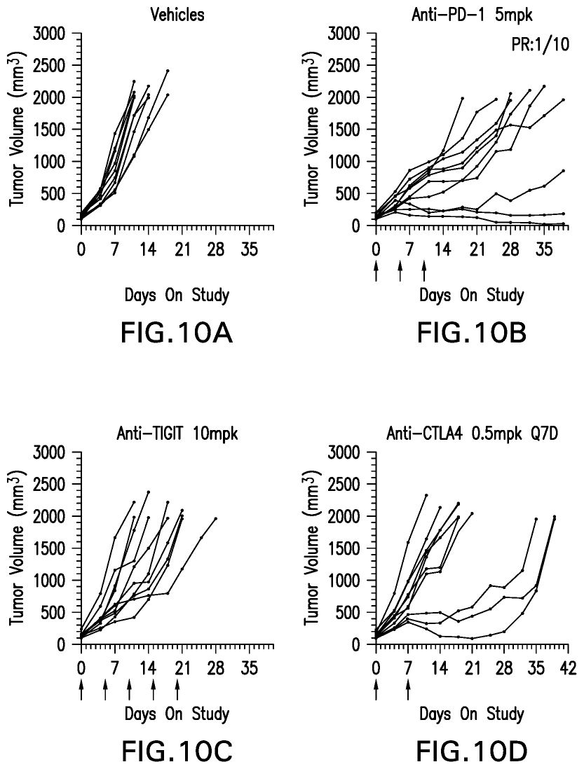

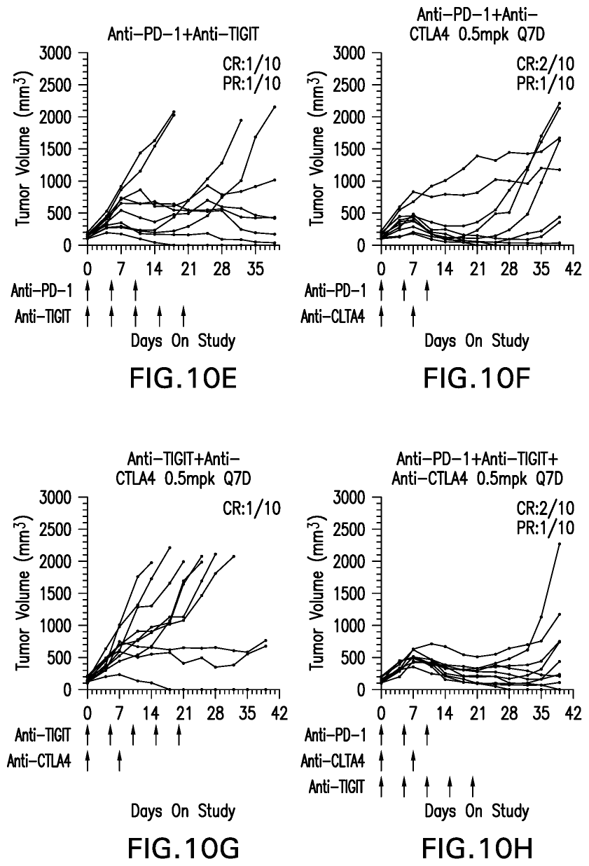

Subjects in the EMT6 tumor model of FIG. 2A were treated with either: vehicle; anti-mouse PD-1 monoclonal antibody muDX400 at a dose of 5 mpk every 5 days for up to 4 separate treatments (i.e., over 20 days) by intraperitoneal injection (5 mpk, Q5D×4, IP); rat-mouse chimeric anti-TIGIT antibody 18G10-IgG2a at a dose of 10 mpk every 5 days for up to 4 separate treatments by intraperitoneal injection (10 mpk, Q5D×4, IP); an anti-mouse CTLA4 antibody (1 mpk, Q5d×4, IP); a combination of anti-PD-1 antibody (5 mpk, Q5D×4, TP); and anti-TIGIT antibody (10 mpk, Q5D×4, IP); a combination of anti-PD-1 antibody (5 mpk, Q5D×4, IP) and anti-CTLA4 antibody (1 mpk, Q5d×4, IP); a combination of anti-TIGIT antibody (10 mpk, Q5D×4, IP) and anti-CTLA4 antibody (1 mpk, Q5D×4, IP); or a combination of anti-PD-1 antibody (5 mpk, Q5D×4, IP), anti-TIGIT antibody (10 mpk, Q5D×4, IP), and anti-CTLA4 antibody (1 mpk, Q5D×4, IP).

Subjects in the EMT6 tumor model of FIG. 2B were treated with either: vehicle; anti-mouse PD-1 monoclonal antibody muDX400 (5 mpk, Q5D×4, IP); rat-mouse chimeric anti-TIGIT antibody 18G10-IgG2a (10 mpk, Q5D×4, IP); an anti-mouse CTLA4 antibody at a dose of 1 mpk every 10 days for up to 2 separate treatments by intraperitoneal injection (1 mpk, Q10D×2, IP); a combination of anti-PD-1 antibody (5 mpk, Q5D×4, IP) and anti-TIGIT antibody (10 mpk, Q5D×4, IP); a combination of anti-PD-1 antibody (5 mpk, Q5D×4, IP) and anti-CTLA4 antibody (1 mpk, Q10D×2, IP); a combination of anti-TIGIT antibody (10 mpk, Q5D×4, IP) and anti-CTLA4 antibody (1 mpk, Q10D×2, IP); or a combination of anti-PD-1 antibody (5 mpk, Q5D×4, IP), anti-TIGIT antibody (10 mpk, Q5D×4, IP), and anti-CTLA4 antibody (1 mpk, Q10D×2, IP).

FIGS. 3A-3H are a series of graphs showing individual tumor volume over a period of days for the murine subjects in FIG. 2A.

FIGS. 4A-4H are a series of graphs showing individual tumor volume over a period of days for the murine subjects in FIG. 2B.

FIG. 5A and FIG. 5B are two graphs showing tumor volume (mm³) and progression as a function of time (days) for murine subjects in a MB49 tumor model treated with murine and/or rat-mouse chimeric antibodies directed to either PD-1, TIGIT, or CTLA4.

Subjects in the MB49 tumor model of FIG. 5A were treated with either: vehicle; anti-mouse PD-1 monoclonal antibody muDX400 (5 mpk, Q5D×4, IP); rat-mouse chimeric anti-TIGIT antibody 18G10-IgG2a (10 mpk, Q5D×4, IP); an anti-mouse CTLA4 antibody (1 mpk, Q5D×4, IP); a combination of anti-PD-1 antibody and anti-TIGIT antibody; a combination of anti-PD-1 antibody (5 mpk, Q5D×4, IP) and anti-TIGIT antibody (10 mpk, Q5D×4, IP); a combination of anti-PD-1 antibody (5 mpk, Q5D×4, IP) and anti-CTLA4 antibody (1 mpk, Q5d×4, IP); a combination of anti-TIGIT antibody (10 mpk, Q5D×4, IP) and anti-CTLA4 antibody (1 mpk, Q5D×4, IP); or a combination of anti-PD-1 antibody (5 mpk, Q5D×4, IP), anti-TIGIT antibody (10 mpk, Q5D×4, IP), and anti-CTLA4 antibody (1 mpk, Q5D×4, IP).

Subjects in the MB49 tumor model of FIG. 5B were treated with either: vehicle; anti-mouse PD-1 monoclonal antibody muDX400 (5 mpk, Q5D×4, IP); rat-mouse chimeric anti-TIGIT antibody 18G10-IgG2a (10 mpk, Q5D×4, IP); an anti-mouse CTLA4 antibody (1 mpk, Q10D×2, IP); a combination of anti-PD-1 antibody (5 mpk, Q5D×4, IP) and anti-TIGIT antibody (10 mpk, Q5D×4, IP); a combination of anti-PD-1 antibody (5 mpk, Q5D×4, IP) and anti-CTLA4 antibody (1 mpk, Q10D×2, IP); a combination of anti-TIGIT antibody (10 mpk, Q5D×4, IP) and anti-CTLA4 antibody (1 mpk, Q10D×2, IP); or a combination of anti-PD-1 antibody (5 mpk, Q5D×4, IP), anti-TIGIT antibody (10 mpk, Q5D×4, TP), and anti-CTLA4 antibody (1 mpk, Q10D×2, IP).

FIGS. 6A-6H are a series of graphs showing individual tumor volume over a period of days for the murine subjects in FIG. 5A.

FIGS. 7A-7H are a series of graphs showing individual tumor volume over a period of days for the murine subjects in FIG. 5B.

FIG. 8A and FIG. 8B are two graphs showing tumor volume (mm³) and progression as a function of time (days) for murine subjects in a MBT2 tumor model treated with murine and/or rat/mouse chimeric antibodies directed to either PD-1, TIGIT, or CTLA4.

Subjects in the MBT2 tumor model of FIG. 8A were treated with either: vehicle; anti-mouse PD-1 monoclonal antibody muDX400 (5 mpk, Q5D×3, IP); rat-mouse chimeric anti-TIGIT antibody 18G10-IgG2a (10 mpk, Q5D×5, IP); an anti-mouse CTLA4 antibody (1 mpk, Q7d×2, IP); a combination of anti-PD-1 antibody (5 mpk, Q5D×3, IP) and anti-TIGIT antibody (10 mpk, Q5D×5, IP); a combination of anti-PD-1 antibody (5 mpk, Q5D×3, IP) and anti-CTLA4 antibody (1 mpk, Q7d×2, IP); a combination of anti-TIGIT antibody (10 mpk, Q5D×5, IP) and anti-CTLA4 antibody (1 mpk, Q7D×2, IP); or a combination of anti-PD-1 antibody (5 mpk, Q5D×3, IP), anti-TIGIT antibody (10 mpk, Q5D×5, IP), and anti-CTLA4 antibody (1 mpk, Q7D×2, IP)

Subjects in the MBT2 tumor model of FIG. 8B were treated with either: vehicle; anti-mouse PD-1 monoclonal antibody muDX400 (5 mpk, Q5D×3, IP); rat-mouse chimeric anti-TIGIT antibody 18G10-IgG2a (10 mpk, Q5D×5, IP); an anti-mouse CTLA4 antibody (0.5 mpk, Q7d×2, IP); a combination of anti-PD-1 antibody (5 mpk, Q5D×3, IP) and anti-TIGIT antibody (10 mpk, Q5D×5, IP); a combination of anti-PD-1 antibody (5 mpk, Q5D×3, IP) and anti-CTLA4 antibody (0.5 mpk, Q7d×2, IP); a combination of anti-TIGIT antibody (10 mpk, Q5D×5, IP) and anti-CTLA4 antibody (0.5 mpk, Q7D×2, IP); or a combination of anti-PD-1 antibody (5 mpk, Q5D×3, IP), anti-TIGIT antibody (10 mpk, Q5D×5, IP), and anti-CTLA4 antibody (0.5 mpk, Q7D×2, IP)

FIGS. 9A-9H are a series of graphs showing individual tumor volume over a period of days for the murine subjects in FIG. 8A.

FIGS. 10A-10H are a series of graphs showing individual tumor volume over a period of days for the murine subjects in FIG. 8n.

### DETAILED DESCRIPTION OF THE INVENTION

### 1. Abbreviations with their Expanded Terms

| Abbreviation | Expanded Term |
|---|---|
| 1L | First line |
| ADA | antidrug antibody |
| ADL | activities of daily living |
| ADR | adverse drug reaction |
| AE | adverse event |
| AJCC | American Joint Committee on Cancer |
| ALT | Alanine aminotransferase |
| anti-CTLA-4 | anticytotoxic T-lymphocyte-associated protein 4 |
| APaT | All-Participants-as-Treated |
| aPTT | Activated partial thromboplastin time |
| AST | Aspartate aminotransferase |
| AUC | area under the curve |
| BCG | Bacillus Calmette-Guérin |
| BICR | blinded independent central review |
| BID | One dose twice daily |
| BP | blood pressure |
| BRAF | v-raf murine sarcoma viral oncogene homolog B1 |
| C1D1 | Cycle 1 Day 1 |
| C57B1/6 | Common inbred strain of laboratory mouse, also “C57 |
| | black 6”, “C57”, “black 6”, or “B6” |
| C/A/P | chest, abdomen, and pelvis |
| C_{avg} | Average concentration over the dosing interval |
| CD | cluster of differentiation |
| CDR | Complementary determining region |
| CD28 | cluster of differentiation 28 |
| CHMP | Committee for Medicinal Products for Human Use |
| | (CHMP) is the European Medicines Agency committee |
| | responsible for human medicines. |
| CHO | Chinese hamster ovary |
| CI | confidence interval |
| CIV | central imaging vendor |
| Cₘₐₓ | This pharmacokinetics term refers to the maximum (or |
| | peak) serum concentration that a drug achieves in a |
| | specified test area of the body after the drug has been |
| | administered, which is the minimum (or trough) |
| | concentration that a drug achieves after dosing. |
| Cₘᵢₙ | This pharmacokinetics term refers to the minimum (or |
| | trough) concentration that a drug achieves in a specified |
| | test area of the body after dosing. |
| CNS | central nervous system |
| CONSORT | Consolidated Standards of Reporting Trials |
| CR | complete response |
| CRF | Case Report Form |
| C-SSRS | Columbia-Suicide Severity Rating Scale |
| CSR | Clinical Study Report |
| CT | computed tomography |
| CTCAE | Common Terminology Criteria for Adverse Events |
| CTCAE 5.0 | Common Terminology Criteria for Adverse Events, |
| | version 5.0 |
| ctDNA | circulating tumor DNA |
| CTFG | clinical trial facilitation group |
| CTLA-4 | cytotoxic T-lymphocyte-associated protein 4 |
| Ctrl | Control |
| DDI | drug-drug interaction |
| DLT | dose-limiting toxicity |
| DMC | data monitoring committee |
| DNA | deoxyribonucleic acid |
| DOR | Duration of Response |
| E-R | Exposure-response |
| ECG | Electrocardiogram |
| ECHO | Echocardiogram |
| ECI | event of clinical interest |
| ECOG | Eastern Cooperative Oncology Group |
| EDC | electronic data capture |
| EFS | event-free survival |
| ELISA | enzyme-linked immunosorbent assay |
| EMT6 | murine mammary carcinoma cell line derived from a |
| | transplanted hyperplastic alveolar nodule of a BALB/c |
| | mouse 1 |
| EMA | European Medicines Agency |
| EORTC | European Organization for Research and Treatment of |
| | Cancer |
| EORTC | European Organization for Research and Treatment of |
| QLQ-30 | Cancer Quality of Life Questionnaire-Core 30 |
| EOT | End of treatment |
| EQ-5D-5L | European Quality of Life 5-dimension 5-level |
| EU | European Union |
| FAS | Full Analysis Set |
| FDA | Food and Drug Administration |
| FDAAA | Food and Drug Administration Amendments Act |
| FGF | fibroblast growth factor |
| FGFR | fibroblast growth factor receptor |
| FR | framework region |
| FSH | follicle stimulating hormone |
| FT4 | free thyroxine |
| G-CSF | Granulocyte Colony-Stimulating Factor |
| GCP | Good Clinical Practice |
| GITR | glucocorticoid-induced TNFR-related protein |
| GVHD | Graft-versus-host disease |
| HBsAg | Hepatitis B surface antigen |
| HBV | Hepatitis B virus |
| HCV | Hepatitis C virus |
| HIV | human immunodeficiency virus |
| HRQoL | health-related quality of life |
| HRT | hormone replacement therapy |
| HUVEC | human umbilical vein endothelial cell |
| IA | interim analysis |
| IB | Investigator’s Brochure |
| IC50 | mean inhibitory concentration |
| ICF | Informed Consent Form |
| ICH | International Council for Harmonisation of Technical |
| | Requirements for Pharmaceuticals for Human Use |
| iCPD | iRECIST confirmed progressive disease |
| iCR | iRECIST complete response |
| IDO | indoleamine-(2,3)-dioxygenase |
| IgG | Immunoglobulin G |
| IgG1 | Immunoglobulin G, subclass 1 |
| IHC | Immunohistochemistry |
| IL-2R | interleukin-2 receptor |
| IMP | Investigational medicinal product |
| IND | Investigational New Drug |
| IO | immuno-oncology |
| INR | International Normalized Ratio |
| IP | Intraperitoneal |
| iPR | iRECIST partial response |
| irAE | immune-related adverse event |
| IRB | Institutional Review Board |
| iRECIST | Response Evaluation Criteria in Solid Tumors 1.1 for |
| | immune-based therapeutics |
| IRT | interactive response technology |
| iSD | iRECIST stable disease |
| IT | intratumoral |
| ITT | immunoglobulin tail tyrosine |
| ITIM | immunoreceptor tyrosine-based inhibitory motif |
| iUPD | iRECIST unconfirmed progressive disease |
| IV | Intravenous |
| IVD | in vitro diagnostic |
| kg | Kilogram |
| KO | knock-out |
| LDH | lactate dehydrogenase |
| LVEF | left ventricular ejection fraction |
| M&S | modeling and simulation |
| mAb | monoclonal antibody |
| MAPK | mitogen-activated protein kinase |
| MB49 | murine bladder-49 tumor cell line |
| MBT2/ | mouse bladder tumor-2 cell line |
| MBT-2 | |
| MedDRA | Medical Dictionary for Regulatory Activities |
| MEK | mitogen-activated protein kinase |
| Mg | Milligram |
| mIgG1 | murine immunoglobulin G subclass 1, Isotype control |
| | mAb for anti-PD-1 antibody muDX400 |
| mL | Milliliter |
| MLR | mixed lymphocyte reaction |
| Mm | Millimeter |
| mm | cubic millimeter, 0.001 mL |
| MPK | milligram per kilogram |
| MRI | magnetic resonance imaging |
| mRNA | messenger RNA |
| MSD | Merck Sharp and Dohme Corp., a subsidiary of |
| | Merck & Co., Inc. |
| MSI | microsatellite instability |
| MTD | maximum tolerated dose |
| mTPI | modified Toxicity Probability Interval |
| MUGA | multiple-gated acquisition |
| N | number of subjects in a treatment group |
| NCI | National Cancer Institute |
| NDA | New Drug Application |
| NE | not estimable |
| NK | Natural killer (cells) |
| NK T | Natural killer T (cells) |
| NSCLC | non-small cell lung cancer |
| OR | overall response |
| ORR | objective response rate |
| OS | overall survival |
| PBPK | physiologically-based PK |
| PBS | phosphate-buffered saline, vehicle control for CXCR2 |
| | antagonists |
| pCR | pathological complete response |
| PD-1 | programmed cell-death 1 |
| PDGFR | platelet-derived growth factor receptor |
| PD-L1 | programmed cell-death ligand 1 |
| PD-L2 | programmed cell-death ligand 2 |
| PET | positron emission tomography |
| PFS | Progression-free survival |
| PgP | P-glycoprotein |
| PK | Pharmacokinetic |
| PO | by mouth (orally) |
| PP | Per Protocol |
| pPR | pathological partial response |
| PR | partial response |
| PRES/ | Posterior Reversible Encephalopathy Syndrome/ |
| RPLS | Reversible Posterior Leukoencephalopathy Syndrome |
| PRO | patient-reported outcome |
| pRR | pathological response rate |
| PT | prothrombin time |
| PTT | partial thromboplastin time |
| p-values | calculated probability |
| PVR | poliovirus receptor |
| PVRL-2 | poliovirus receptor-related 2 |
| Q2W | every 2 weeks |
| Q3W | every 3 weeks |
| Q6W | every 6 weeks |
| QD | once daily/one dose per day |
| Q5D | dose every 5 days |
| Q7D | dose every 7 days |
| Q10D | dose every 10 days |
| QLQ-C30 | Quality of Life Questionnaire-Core 30 |
| QoL | quality of life |
| QT | the time between the beginning of the QRS complex and |
| | the end of the T-wave |
| QTc | The QTc interval is the length of time it take the |
| | electrical system in the heart to repolarize, adjusted |
| | for heart rate. |
| RCC | renal cell carcinoma |
| RCF | relative centrifugal force |
| RECIST | Response Evaluation Criteria in Solid Tumors |
| RECIST 1.1 | Response Evaluation Criteria in Solid Tumors 1.1 |
| RENCA | Renal cell carcinoma |
| RFS | recurrence-free survival |
| RNA | ribonucleic acid |
| RP2D | recommended Phase 2 dose |
| RTKi | receptor tyrosine kinase inhibitor |
| SAC | Scientific Advisory Committee |
| SAE | serious adverse event |
| SAP | Statistical Analysis Plan |
| SD | stable disease |
| SEM | standard error of the mean |
| SHP-1 | Src homology 2 domain-containing protein tyrosine |
| | phosphatase 1 |
| SHP-2 | Src homology 2 domain-containing protein tyrosine |
| | phosphatase 2 |
| SIM | Site Imaging Manual |
| SNP | single-nucleotide polymorphism |
| SoA | Schedule of Activities |
| SOC | standard-of-care |
| sSAP | supplemental Statistical Analysis Plan |
| T3 | Triiodothyronine |
| T/C | median tumor volume of the treated animal/Median |
| | tumor volume of the control animal |
| TCID | tissue culture infectious dose |
| Teffs | effector T-cells |
| TGI | tumor growth inhibition |
| Tregs | regulatory T-cells |
| TSH | thyroid-stimulating hormone |
| TTD | time to true deterioration |
| μg, ug, UG | Microgram |
| ULN | upper limit of normal |
| UPCR | urine protein-to-creatinine ratio |
| US | United States |
| VEGF | vascular endothelial growth factor |
| VEGFR | vascular endothelial growth factor receptors |
| V_{H} | immunoglobulin heavy chain variable region |
| V_{K} | immunoglobulin kappa light chain variable region |
| V_{L} | immunoglobulin light chain variable region |
| WOCBP | woman/women of childbearing potential |

Additional abbreviations may be defined throughout this disclosure.

### 2. Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter (e.g., the dose of an anti-PD-1 antibody or antigen binding fragment thereof, an anti-CTLA4 antibody or antigen binding fragment thereof, or an anti-TIGIT antibody or antigen binding fragment thereof, or the length of treatment time with a combination therapy described herein) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number. For example, a dose of about 2 mg/kg may vary between 1.6 mg/kg and 2.4 mg/kg. In certain embodiments, a dose of about 25 mg may vary between 20 mg and 30 mg. In certain embodiments, a dose of about 200 mg may vary between 160 mg and 240 mg. In certain embodiments, a dose of about 400 mg may vary between 320 mg and 480 mg.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-PD-1 antibody, an anti-CTLA4 antibody, and an anti-TIGIT antibody as described herein) into a subject (e.g., a patient), such as by mucosal, intradermal, intravenous, intramuscular delivery, and/or any other methods of physical delivery described herein or known in the art. For example, the anti-PD-1 antibody, the anti-CTLA4 antibody, and the anti-TIGIT antibody are each administered intravenously or by infusion.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.” Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} f-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” or “antigen binding fragment thereof” refers to a fragment of an antibody that retains the ability to bind specifically to the antigen, e.g., fragments that retain one or more CDR regions. An antibody that “specifically binds to” PD-1, CTLA4, or TIGIT is an antibody that exhibits preferential binding to PD-1, CTLA4, or TIGIT (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., PD-1, CTLA4, or TIGIT). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The term “pharmaceutically acceptable carrier” refers to any inactive substance that is suitable for use in a formulation for the delivery of a therapeutic agent. A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorphs (e.g., rabbits), rodents (e.g., rats or mice), Procyon lotor (e.g., raccoons). In particular embodiments, the subject is human. For example, the human subject is suffering from cancer, an infection or an infectious disease as defined herein.

The term “subject in need thereof” as used herein refers to a subject diagnosed with or suspected of having cancer or an infectious disease as defined herein.

“Biotherapeutic agent” means a biological molecule, such as an antibody or fusion protein, that blocks ligand/receptor signaling in any biological pathway that supports tumor maintenance and/or growth or suppresses the anti-tumor immune response.

“Chemotherapeutic agent” refers to a chemical or biological substance that can cause death of cancer cells, or interfere with growth, division, repair, and/or function of cancer cells. Examples of chemotherapeutic agents include those that are disclosed in WO2006/129163, and US20060153808. Classes of chemotherapeutic agents include, but are not limited to: alkylating agents, antimetabolites, kinase inhibitors, spindle poison, plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell-proliferation or tumor growth. Chemotherapeutic agents useful in the treatment methods of the present disclosure include cytostatic and/or cytotoxic agents.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-PD-1 antibody, an anti-CTLA4 antibody, or an anti-TIGIT antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-PD-1 antibody variant, an anti-CTLA4 antibody variant, or an anti-TIGIT antibody variant) at least retains the antibody functional activity. In specific embodiments, an anti-PD-1 antibody variant binds to PD-1 and/or is antagonistic to PD-1 activity. In some embodiments, an anti-CTLA4 antibody variant binds to CTLA4 and/or is antagonistic to CTLA4 activity. In some embodiments, an anti-TIGIT antibody variant binds to TIGIT and/or is antagonistic to TIGIT activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

“Anti-CTLA4 antibody” means a monoclonal antibody that specifically binds to human CTLA4. Human CTLA4 comprises the amino acid sequence:

(SEQ ID NO: 112)
AMHVAQPAVVLASSRGIASFVCEYASPGKATEVRVTVLRQADSQVTEVCA
ATYMMGNELTFLDDSICTGTSSGNQVNLTIQGLRAMDTGLYICKVELMYP
PPYYLGIGNGTQIYVIDPEPCPDSD

Therefore, in the present invention, the term “CTLA4 protein” should include all such sequences, including the sequence as set forth in SEQ ID NO: 112, as well as its native or artificial variants. Furthermore, when reference is made to a sequence fragment of the CTLA4 protein, it not only includes a sequence fragment of SEQ ID NO: 112, but also includes the corresponding sequence fragments of its native or artificial variants.

As used herein, the term “effective amount” refer to an amount of an anti-PD-1 antibody or antigen binding fragment, an anti-TIGIT or antigen-binding fragment thereof, and/or an anti-CTLA4 antibody or antigen binding fragment of the invention (e.g., described in Table 16) that, when administered alone or in combination with an additional therapeutic agent to a cell, tissue, or subject, is effective to cause a measurable improvement in one or more symptoms of an infection or a disease, for example cancer or the progression of cancer. An effective dose further refers to that amount of the antibody or fragment sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic may result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. Toxicity and therapeutic efficacy of the antibodies or antigen binding fragments of the invention, administered alone or in combination with another therapeutic agent, can be determined by any number of systems or means. For example, the toxicity and therapeutic efficacy of the antibodies or antigen binding fragments of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“Anti-PD-1 antibody” means a monoclonal antibody that specifically binds to human PD-1. PD-1 is recognized as an important molecule in immune regulation and the maintenance of peripheral tolerance. PD-1 is moderately expressed on naive T, B and NKT cells and up-regulated by T/B cell receptor signaling on lymphocytes, monocytes and myeloid cells (Sharpe, A. H, Wherry, E. J., Ahmed R., and Freeman G. J. The function of programmed cell death 1 and its ligands in regulating autoimmunity and infection. Nature Immunology (2007); 8:239-245). Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (Dong H et al. Nat Med. 2002 August; 8(8):793-800; Yang et al. Invest Ophthalmol Vis Sci. 2008 June; 49(6 (2008): 49: 2518-2525; Ghebeh et al. Neoplasia (2006) 8: 190-198; Hamanishi J et al. Proceeding of the National Academy of Sciences (2007): 104: 3360-3365; Thompson R H et al. Clinical genitourin Cancer (2006): 5: 206-211; Nomi, T. Sho, M., Akahori, T., et al. Clinical Cancer Research (2007); 13:2151-2157; Ohigashi Y et al. Clin. Cancer Research (2005): 11: 2947-2953; Inman et al. Cancer (2007): 109: 1499-1505; Shimauchi T et al. Int. J. Cancer (2007): 121:2585-2590; Gao et al. Clinical Cancer Research (2009) 15: 971-979; Nakanishi J., Cancer Immunol Immunother. (2007) 56: 1173-1182; Hino et al., Cancer (2010): 00: 1-9.). Similarly, PD-1 expression on tumor infiltrating lymphocytes was found to mark dysfunctional T cells in breast cancer and melanoma (Ghebeh H., BMC Cancer. 2008 Feb. 23; 8:57; and Ahmadzadeh M et al. Blood (2009) 114: 1537-1544) and to correlate with poor prognosis in renal cancer (Thompson R H et al, Clinical Cancer Research (2007) 15: 1757-1761). Thus, it has been proposed that PD-L1 expressing tumor cells interact with PD-1 expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 have been approved for treating cancer. Pembrolizumab is a potent humanized immunoglobulin G4 (IgG4) mAb with high specificity of binding to the programmed cell death 1 (PD 1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1.

Keytruda® (pembrolizumab) is indicated for the treatment of patients across a number of indications.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

“Treat” or “treating” cancer as used herein means to administer a therapeutic combination of an anti-human PD-1 monoclonal antibody or antigen binding fragment thereof, an anti-human CTLA4 monoclonal antibody or antigen binding fragment thereof, and an anti-human TIGIT monoclonal antibody or antigen binding fragment thereof, to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth. Such “treatment” may result in a slowing, interrupting, arresting, controlling, or stopping of the progression of cancer as described herein but does not necessarily indicate a total elimination of the cancer or the symptoms of the cancer. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treat” or “treating” an infection and/or an infectious disease means to administer a therapeutic agent, such as a composition containing any of the antibodies or antigen-binding fragments of the present invention, internally or externally to a subject or patient having one or more disease symptoms of the infection or the infectious disease, or being suspected of having the infection and/or the infectious disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

As used herein, the terms “combination therapy” and “therapeutic combination” refer to treatments in which at least one anti-human PD-1 monoclonal antibody or antigen-binding fragment thereof, at least one anti-human CTLA4 monoclonal antibody or antigen-binding fragment thereof, and at least one anti-human TIGIT monoclonal antibody or antigen-binding fragment thereof, and optionally additional therapeutic agents, each are administered to a patient in a coordinated manner, over an overlapping period of time. The period of treatment with the at least one anti-human PD-1 monoclonal antibody (or antigen-binding fragment thereof) (the “anti-PD-1 treatment”) is the period of time that a patient undergoes treatment with the anti-human PD-1 monoclonal antibody (or antigen-binding fragment thereof); that is, the period of time from the initial dosing with the anti-human PD-1 monoclonal antibody (or antigen-binding fragment thereof) through the final day of a treatment cycle. Similarly, the period of treatment with the at least one anti-human CTLA4 monoclonal antibody (or antigen-binding fragment thereof) (the “anti-CTLA4 treatment”) is the period of time that a patient undergoes treatment with the anti-human CTLA4 monoclonal antibody (or antigen-binding fragment thereof); that is, the period of time from the initial dosing with the anti-human CTLA4 monoclonal antibody (or antigen-binding fragment thereof) through the final day of a treatment cycle. The period of treatment with the at least one anti-human TIGIT monoclonal antibody (or antigen-binding fragment thereof) (the “anti-TIGIT treatment”) is the period of time that a patient undergoes treatment with the anti-human TIGIT monoclonal antibody (or antigen-binding fragment thereof); that is, the period of time from the initial dosing with the anti-human TIGIT monoclonal antibody (or antigen-binding fragment thereof) through the final day of a treatment cycle. In the methods and therapeutic combinations described herein, the anti-PD-1 treatment overlaps by at least one day with the anti-CTLA4 treatment and overlaps by at least one day with the anti-TIGIT treatment. In certain embodiments, the anti-PD-1 treatment, the anti-CTLA4 treatment, and the anti-TIGIT treatment are the same period of time. In some embodiments, the anti-PD-1 treatment begins prior to the anti-CTLA4 and/or the anti-TIGIT treatment. In other embodiments, the anti-PD-1 treatment begins after the anti-CTLA4 and/or the anti-TIGIT treatment. In yet other embodiments, the anti-CTLA4 treatment begins prior to the anti-PD-1 and/or the anti-TIGIT treatment. In still other embodiments, the anti-CTLA4 treatment begins after the anti-PD-1 and/or the anti-TIGIT treatment. In some embodiments, the anti-TIGIT treatment begins prior to the anti-CTLA4 and/or the anti-PD-1 treatment. In other embodiments, the anti-TIGIT treatment begins after the anti-CTLA4 and/or the anti-PD-1 treatment. In certain embodiments, the anti-PD-1 treatment is terminated prior to termination of the anti-CTLA4 and/or the anti-TIGIT treatment. In other embodiments, the anti-PD-1 treatment is terminated after termination of the anti-CTLA4 and/or the anti-TIGIT treatment. In yet other embodiments, the anti-CTLA4 treatment is terminated prior to termination of the anti-PD-1 and/or the anti-TIGIT treatment. In still other embodiments, the anti-CTLA4 treatment is terminated after termination of the anti-PD-1 and/or the anti-TIGIT treatment. In certain embodiments, the anti-TIGIT treatment is terminated prior to termination of the anti-CTLA4 and/or the anti-PD-1 treatment. In other embodiments, the anti-TIGIT treatment is terminated after termination of the anti-CTLA4 and/or the anti-PD-1 treatment.

Auxiliary or supplementary therapeutic agents and/or active agents (e.g., compounds or molecules) can also be incorporated into the methods, compositions, kits and uses described herein. In certain embodiments, the claimed treatment (i.e., administering the anti-PD-1 antibody or antigen-binding fragment thereof, the anti-CTLA4 antibody or antigen-binding fragment thereof, and the anti-TIGIT antibody or antigen-binding fragment thereof) disclosed herein is coformulated with and/or coadministered with one or more additional therapeutic agents and/or active agents that are useful for treating disorders. For example, the anti-PD-1 antibody or antigen-binding fragment thereof, the anti-CTLA4 antibody or antigen-binding fragment thereof, and the anti-TIGIT antibody or antigen-binding fragment thereof disclosed herein may be coformulated and/or coadministered with one or more additional therapeutic agents and/or active agents that bind other targets (e.g., antibodies that bind other cytokines, cancer antigens, or that bind cell surface molecules). Furthermore, one or more binding proteins disclosed herein may be used in combination with two or more of the foregoing therapeutic agents and/or active agents: a second antibody or fragment thereof, an immunomodulator, a hormone, a cytotoxic agent, an enzyme, a radionuclide, a second antibody conjugated to at least one immunomodulator, enzyme, radioactive label, hormone, antisense oligonucleotide, biotherapeutic, chemotherapeutic, cytotoxic agent, or a combination thereof. In an embodiment, the one or more additional therapeutic agents and/or active agents comprise at least one from the group consisting of: an imaging agent; a cytotoxic agent; an angiogenesis inhibitor; a kinase inhibitor; a co-stimulation molecule blocker; an adhesion molecule blocker; an anti-cytokine antibody or functional fragment thereof; methotrexate; cyclosporin; rapamycin; FK506; a detectable label or reporter; a narcotic, a non-steroid anti-inflammatory drug (NSAID); an analgesic; an anesthetic; a sedative; a local anesthetic; a neuromuscular blocker; an antimicrobial; an antipsoriatic; a corticosteroid; an erythropoietin; an immunization; an immunoglobulin; a hormone replacement drug; a radiopharmaceutical; a cytokine; and a cytokine antagonist. Such combination therapies (comprising one or more additional therapeutic agents and/or active agents) may advantageously utilize lower dosages of the administered therapeutic agents, thus improving efficacy and/or avoiding possible toxicities or complications associated with the treatment.

The terms “treatment regimen,” “dosing protocol,” and “dosing regimen” are used interchangeably to refer to the dose and timing of administration of each therapeutic agent in a combination therapy of the disclosure.

“Anti-TIGIT antibody” means a monoclonal antibody that specifically binds to human TIGIT. Human TIGIT comprises the amino acid sequence:

(SEQ ID NO: 111)
MRWCLLLIWA QGLRQAPLAS GMMTGTIETT GNISAEKGGS
IILQCHLSST TAQVTQVNWE QQDQLLAICN ADLGWHISPS
FKDRVAPGPG LGLTLQSLTV NDTGEYFCIY HTYPDGTYTG
RIFLEVLESS VAEHGARFQI PLLGAMAATL VVICTAVIVV
VALTRKKKAL RIHSVEGDLR RKSAGQEEWS PSAPSPPGSC
VQAEAAPAGL CGEQRGEDCA ELHDYFNVLS YRSLGNCSFF
TETG;

See also amino acid residues 25-244 of Genbank Accession Number NP_776160.2 (SEQ ID NO: 111) (amino acid residues 1-24 of SEQ ID NO:111 correspond to a leader peptide).

Therefore, in the present invention, the term “TIGIT protein” should include all such sequences, including the sequence as set forth in SEQ ID NO: 111, as well as its native or artificial variants.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

It is understood that wherever embodiments are described herein with the language “comprising,” otherwise analogous embodiments described in terms of “consisting of” and/or “consisting essentially of” are also provided.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, +2%, ±3%, ±4%, +5%, +10%, +15%, and ±20% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

Embodiment 1. A method of treating cancer, an infection disease or an infection comprising administering to a subject in need thereof effective amounts of:

(a) an anti-human PD-1 monoclonal antibody or antigen binding fragment thereof,

(b) an anti-human CTLA4 monoclonal antibody or antigen binding fragment thereof; and

(c) an anti-human TIGIT monoclonal antibody or antigen binding fragment thereof.

Embodiment 2. The method of embodiment 1, wherein the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.
Embodiment 3. A method of enhancing T cell activity, comprising contacting the T cells with:

(a) an anti-human PD-1 monoclonal antibody or antigen binding fragment thereof;

(b) an anti-human CTLA4 monoclonal antibody or antigen binding fragment thereof; and

(c) an anti-human TIGIT monoclonal antibody or antigen binding fragment thereof.

Embodiment 4. A method of increasing cytokine production of T cells, comprising contacting the T cells with:

(a) an anti-human PD-1 monoclonal antibody or antigen binding fragment thereof;

(b) an anti-human CTLA4 monoclonal antibody or antigen binding fragment thereof; and

(c) an anti-human TIGIT monoclonal antibody or antigen binding fragment thereof.

Embodiment 5. The method of embodiment 4, wherein the cytokine is selected from the group consisting of IL-1, IL-2, IL-6, IL-12, IL-17, IL-22, IL-23, GM-CSF, IFN-α, and TNF-α.
Embodiment 6. A method of increasing proliferation of T cells, comprising contacting the T cells with:

(a) an anti-human PD-1 monoclonal antibody or antigen binding fragment thereof;

(b) an anti-human CTLA4 monoclonal antibody or antigen binding fragment thereof; and

(c) an anti-human TIGIT monoclonal antibody or antigen binding fragment thereof.

Embodiment 7. A pharmaceutical composition for treating cancer, an infection disease or an infection in a subject in need thereof comprising:

(a) an anti-human PD-1 monoclonal antibody or antigen binding fragment thereof;

(b) an anti-human CTLA4 monoclonal antibody or antigen binding fragment thereof; and

(c) an anti-human TIGIT monoclonal antibody or antigen binding fragment thereof.

Embodiment 8. The pharmaceutical composition of embodiment 7, further comprising a pharmaceutically acceptable carrier.
Embodiment 9. A kit for treating cancer, an infection disease or an infection in a subject in need thereof comprising:

(a) an anti-human PD-1 monoclonal antibody or antigen binding fragment thereof;

(b) an anti-human CTLA4 monoclonal antibody or antigen binding fragment thereof; and

(c) an anti-human TIGIT monoclonal antibody or antigen binding fragment thereof.

Embodiment 10. The kit of embodiment 9, further comprising instructions for administering to a subject in need thereof effective amounts of the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.
Embodiment 11. Use of a therapeutic combination for treating cancer, an infection disease or an infection in a subject in need thereof, wherein the therapeutic combination comprises effective amounts of:

(a) an anti-human PD-1 monoclonal antibody or antigen binding fragment thereof;

(b) an anti-human CTLA4 monoclonal antibody or antigen binding fragment thereof; and

(c) an anti-human TIGIT monoclonal antibody or antigen binding fragment thereof.

Embodiment 12. The use of embodiment 11, wherein the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.
Embodiment 13. The method, pharmaceutical composition, kit, or use of any one of embodiments 1-12, wherein the subject is a human patient.
Embodiment 14. The method, pharmaceutical composition, kit, or use of any one of embodiments 1-13 for treating cancer.
Embodiment 15. The method, pharmaceutical composition, kit, or use of any one of embodiments 1-14, wherein the anti-human PD-1 monoclonal antibody is a human antibody.
Embodiment 16. The method, pharmaceutical composition, kit, or use of any one of embodiments 1-14, wherein the anti-human PD-1 monoclonal antibody is a humanized antibody.
Embodiment 17. The method, pharmaceutical composition, kit, or use of any one of embodiments 1-16, wherein the anti-human CTLA4 monoclonal antibody is a human antibody.
Embodiment 18. The method, pharmaceutical composition, kit, or use of any one of embodiments 1-16, wherein the anti-human CTLA4 monoclonal antibody is a humanized antibody.
Embodiment 19. The method, pharmaceutical composition, kit, or use of any one of embodiments 1-18, wherein the anti-human TIGIT monoclonal antibody is a human antibody.
Embodiment 20. The method, pharmaceutical composition, kit, or use of any one of embodiments 1-18, wherein the anti-human TIGIT monoclonal antibody is a humanized antibody.
Embodiment 21. The method, pharmaceutical composition, kit, or use of any one of embodiments 1-14, wherein the anti-human PD-1 monoclonal antibody or antigen binding fragment thereof comprises a light chain variable region V_{L} complementarity determining region 1 (CDR1), a V_{L} CDR2, and a V_{L} CDR3 comprising amino acid sequences as set forth in SEQ ID NOS:1, 2, and 3, respectively, and a heavy chain variable region V_{H} CDR1, a V_{H} CDR2, and a V_{H} CDR3 comprising amino acid sequences as set forth in SEQ ID NOS:6, 7, and 8, respectively.
Embodiment 22. The method, pharmaceutical composition, kit, or use of any of embodiments 1-14, wherein the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.
Embodiment 23. The method, pharmaceutical composition, kit, or use of any of embodiments 1-14, wherein the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.
Embodiment 24. The method, pharmaceutical composition, kit, or use of any one of embodiments 1-14, wherein the anti-human PD-1 monoclonal antibody or antigen binding fragment thereof comprises a light chain variable region V_{L} complementarity determining region 1 (CDR1), a V_{L} CDR2, and a V_{L} CDR3 comprising amino acid sequences as set forth in SEQ ID NOS:31, 32, and 33, respectively, and a heavy chain variable region V_{H} CDR1, a V_{H} CDR2, and a V_{H} CDR3 comprising amino acid sequences as set forth in SEQ ID NOS:36, 37, and 38, respectively.
Embodiment 25. The method, pharmaceutical composition, kit, or use of any of embodiments 1-14, wherein the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39.
Embodiment 26. The method, pharmaceutical composition, kit, or use of any of embodiments 1-14, wherein the anti-human PD-1 monoclonal antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40.
Embodiment 27. The method, pharmaceutical composition, kit, or use of any of embodiments 1-14, wherein the anti-human PD-1 monoclonal antibody or antigen binding fragment thereof is pembrolizumab.
Embodiment 28. The method, pharmaceutical composition, kit, or use of any of embodiments 1-14, wherein the anti-human PD-1 monoclonal antibody or antigen binding fragment thereof is nivolumab.
Embodiment 29. The method, pharmaceutical composition, kit, or use of any of embodiments 1-14, wherein the anti-human PD-1 monoclonal antibody or antigen binding fragment thereof is cemiplimab.
Embodiment 30. The method, pharmaceutical composition, kit, or use of any of embodiments 1-14, wherein the anti-human CTLA4 monoclonal antibody or antigen binding fragment thereof comprises:

(a) a V_{L} CDR1 comprising the amino acid sequence of SEQ ID NO: 14, a V_{L} CDR2 comprising the amino acid sequence of SEQ ID NO: 15, and a V_{L} CDR3 comprising the amino acid sequences as set forth in SEQ ID NOS:16, 17, or 18, and a V_{H} CDR1, a V_{H} CDR2, and a V_{H} CDR3 comprising amino acid sequences as set forth in SEQ ID NOS:11, 12, and 13, respectively; or

(b) a V_{L} CDR1, a V_{L} CDR2, and a V_{L} CDR3 comprising the amino acid sequences as set forth in SEQ ID NOS:54, 55, and 56, respectively, and a V_{H} CDR1, a V_{H} CDR2, and a V_{H} CDR3 comprising amino acid sequences as set forth in SEQ ID NOS:57, 105, and 106, respectively.

Embodiment 31. The method, pharmaceutical composition, kit, or use of any of embodiments 1-12, wherein the anti-human CTLA4 monoclonal antibody or antigen binding fragment thereof comprises:

(a) a V_{L} region comprising an amino acid sequence as set forth in SEQ ID NO:20, and a V_{H} region comprising an amino acid sequence as set forth in SEQ ID NO:19;

(b) a V_{L} region comprising an amino acid sequence as set forth in SEQ ID NO:42, and a V_{H} region comprising an amino acid sequence as set forth in SEQ ID NO:41;

(c) a V_{L} region comprising an amino acid sequence as set forth in SEQ ID NO:44, and a V_{H} region comprising an amino acid sequence as set forth in SEQ ID NO: 43;

(d) a V_{L} region comprising an amino acid sequence as set forth in SEQ ID NO:44, and a V_{H} region comprising an amino acid sequence as set forth in SEQ ID NO: 45;

(e) a V_{L} region comprising an amino acid sequence as set forth in SEQ ID NO:47, and a V_{H} region comprising an amino acid sequence as set forth in SEQ ID NO:46;

(f) a V_{L} region comprising an amino acid sequence as set forth in SEQ ID NO:49, and a V_{H} region comprising an amino acid sequence as set forth in SEQ ID NO:48;

(g) a V_{L} region comprising an amino acid sequence as set forth in SEQ ID NO:51, and a V_{H} region comprising an amino acid sequence as set forth in SEQ ID NO:50; or

(h) a V_{L} region comprising an amino acid sequence as set forth in SEQ ID NO:108, and a V_{H} region comprising an amino acid sequence as set forth in SEQ ID NO:107.

Embodiment 32. The method, pharmaceutical composition, kit, or use of any of embodiments 1-14, wherein the anti-human CTLA4 monoclonal antibody or antigen binding fragment thereof comprises:

(a) a light chain comprising or consisting of an amino acid sequence as set forth in SEQ ID NO:53 and a heavy chain comprising or consisting of an amino acid sequence as set forth in SEQ ID NO:52; or

(b) a light chain comprising or consisting of an amino acid sequence as set forth in SEQ ID NO:110 and a heavy chain comprising or consisting of an amino acid sequence as set forth in SEQ ID NO:109.

Embodiment 33. The method, pharmaceutical composition, kit, or use of any of embodiments 1-14, wherein the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.
Embodiment 34. The method, pharmaceutical composition, kit, or use of any of embodiments 1-14, wherein the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.
Embodiment 35. The method, pharmaceutical composition, kit, or use of any of embodiments 1-14, wherein the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.
Embodiment 36. The method, pharmaceutical composition, kit, or use of any of embodiments 1-14, wherein:

(a) the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;

(b) the anti-human CTLA4 monoclonal antibody or antigen binding fragment thereof comprises a V_{L} CDR1 comprising the amino acid sequence of SEQ ID NO: 14, a V_{L} CDR2 comprising the amino acid sequence of SEQ ID NO: 15, and a V_{L} CDR3 comprising the amino acid sequences as set forth in SEQ ID NOS:16, 17, or 18, and a V_{H} CDR1, a V_{H} CDR2, and a V_{H} CDR3 comprising amino acid sequences as set forth in SEQ ID NOS:11, 12, and 13, respectively; and

(c) the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

Embodiment 37. The method, pharmaceutical composition, kit, or use of any of embodiments 1-14, wherein:

(a) the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;

(b) the anti-human CTLA4 monoclonal antibody or antigen binding fragment thereof comprises a V_{L} CDR1, a V_{L} CDR2, and a V_{L} CDR3 comprising the amino acid sequences as set forth in SEQ ID NOS:54, 55, and 56, respectively, and a V_{H} CDR1, a V_{H} CDR2, and a V_{H} CDR3 comprising amino acid sequences as set forth in SEQ ID NOS:57, 105, and 106, respectively; and

(c) the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

Embodiment 38. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;

(b) the anti-human CTLA4 monoclonal antibody or antigen binding fragment thereof comprises a V_{L} region comprising an amino acid sequence as set forth in SEQ ID NO:20, and a V_{H} region comprising an amino acid sequence as set forth in SEQ ID NO:19; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 39. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;

(b) the anti-human CTLA4 monoclonal antibody or antigen binding fragment thereof comprises a V_{L} region comprising an amino acid sequence as set forth in SEQ ID NO:44, and a V_{H} region comprising an amino acid sequence as set forth in SEQ ID NO:43; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 40. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;

(b) the anti-human CTLA4 monoclonal antibody or antigen binding fragment thereof comprises a V_{L} region comprising an amino acid sequence as set forth in SEQ ID NO:42, and a V_{H} region comprising an amino acid sequence as set forth in SEQ ID NO:41; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 41. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;

(b) the anti-human CTLA4 monoclonal antibody or antigen binding fragment thereof comprises a V_{L} region comprising an amino acid sequence as set forth in SEQ ID NO:44, and a V_{H} region comprising an amino acid sequence as set forth in SEQ ID NO: NO:45; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 42. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;

(b) the anti-human CTLA4 monoclonal antibody or antigen binding fragment thereof comprises a V_{L} region comprising an amino acid sequence as set forth in SEQ ID NO:47, and a V_{H} region comprising an amino acid sequence as set forth in SEQ ID NO:46; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 43. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;

(b) the anti-human CTLA4 monoclonal antibody or antigen binding fragment thereof comprises a V_{L} region comprising an amino acid sequence as set forth in SEQ ID NO:49, and a V_{H} region comprising an amino acid sequence as set forth in SEQ ID NO:48; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 44. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;

(b) the anti-human CTLA4 monoclonal antibody or antigen binding fragment thereof comprises a V_{L} region comprising an amino acid sequence as set forth in SEQ ID NO:51, and a V_{H} region comprising an amino acid sequence as set forth in SEQ ID NO:50; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 45. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9;

(b) the anti-human CTLA4 monoclonal antibody or antigen binding fragment thereof comprises a V_{L} region comprising an amino acid sequence as set forth in SEQ ID NO:108, and a V_{H} region comprising an amino acid sequence as set forth in SEQ ID NO:107; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 46. The method, pharmaceutical composition, kit, or use of any of embodiments 1-14, wherein:

(a) the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10;

(b) the anti-human CTLA4 monoclonal antibody or antigen binding fragment thereof comprises a light chain comprising or consisting of an amino acid sequence as set forth in SEQ ID NO:53 and a heavy chain comprising or consisting of an amino acid sequence as set forth in SEQ ID NO:52; and

(c) the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

Embodiment 47. The method, pharmaceutical composition, kit, or use of any of embodiments 1-14, wherein:

(a) the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10;

(b) the anti-human CTLA4 monoclonal antibody or antigen binding fragment thereof comprises a light chain comprising or consisting of an amino acid sequence as set forth in SEQ ID NO:110 and a heavy chain comprising or consisting of an amino acid sequence as set forth in SEQ ID NO:109; and

(c) the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

Embodiment 48. The method, pharmaceutical composition, kit, or use of any of embodiments 1-14, wherein:

(a) the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively;

(b) a V_{L} CDR1 comprising the amino acid sequence of SEQ ID NO: 14, a V_{L} CDR2 comprising the amino acid sequence of SEQ ID NO: 15, and a V_{L} CDR3 comprising the amino acid sequences as set forth in SEQ ID NOS:16, 17, or 18, and a V_{H} CDR1, a V_{H} CDR2, and a V_{H} CDR3 comprising amino acid sequences as set forth in SEQ ID NOS:11, 12, and 13, respectively; and

(c) the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

Embodiment 49. The method, pharmaceutical composition, kit, or use of any of embodiments 1-14, wherein:

(a) the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively;

(b) a V_{L} CDR1, a V_{L} CDR2, and a V_{L} CDR3 comprising the amino acid sequences as set forth in SEQ ID NOS:54, 55, and 56, respectively, and a V_{H} CDR1, a V_{H} CDR2, and a V_{H} CDR3 comprising amino acid sequences as set forth in SEQ ID NOS:57, 105, and 106, respectively; and

(c) the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

Embodiment 50. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;

(b) the anti-human CTLA4 monoclonal antibody or antigen binding fragment thereof comprises a V_{L} region comprising an amino acid sequence as set forth in SEQ ID NO:20, and a V_{H} region comprising an amino acid sequence as set forth in SEQ ID NO:19; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 51. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;

(b) the anti-human CTLA4 monoclonal antibody or antigen binding fragment thereof comprises a a V_{L} region comprising an amino acid sequence as set forth in SEQ ID NO:42, and a V_{H} region comprising an amino acid sequence as set forth in SEQ ID NO:41; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 52. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;

(b) the anti-human CTLA4 monoclonal antibody or antigen binding fragment thereof comprises a V_{L} region comprising an amino acid sequence as set forth in SEQ ID NO:44, and a V_{H} region comprising an amino acid sequence as set forth in SEQ ID NO:43; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 53. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;

(b) the anti-human CTLA4 monoclonal antibody or antigen binding fragment thereof comprises a V_{L} region comprising an amino acid sequence as set forth in SEQ ID NO:44, and a V_{H} region comprising an amino acid sequence as set forth in SEQ ID NO: 45 or SEQ ID NO: 45; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29

Embodiment 54. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;

(b) the anti-human CTLA4 monoclonal antibody or antigen binding fragment thereof comprises a V_{L} region comprising an amino acid sequence as set forth in SEQ ID NO:47, and a V_{H} region comprising an amino acid sequence as set forth in SEQ ID NO:46; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 55. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;

(b) the anti-human CTLA4 monoclonal antibody or antigen binding fragment thereof comprises a V_{L} region comprising an amino acid sequence as set forth in SEQ ID NO:49, and a V_{H} region comprising an amino acid sequence as set forth in SEQ ID NO:48; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 56. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;

(b) the anti-human CTLA4 monoclonal antibody or antigen binding fragment thereof comprises a V_{L} region comprising an amino acid sequence as set forth in SEQ ID NO:51, and a V_{H} region comprising an amino acid sequence as set forth in SEQ ID NO:50; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 57. The method, pharmaceutical composition, kit, or use of any of embodiments 1-14, wherein:

(a) the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39;

(b) the anti-human CTLA4 monoclonal antibody or antigen binding fragment thereof comprises a V_{L} region comprising an amino acid sequence as set forth in SEQ ID NO:108, and a V_{H} region comprising an amino acid sequence as set forth in SEQ ID NO:107; and

(c) the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

Embodiment 58. The method, pharmaceutical composition, kit, or use of any of embodiments 1-14, wherein:

(a) the anti-human PD-1 monoclonal antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40;

(b) the anti-human CTLA4 monoclonal antibody or antigen binding fragment thereof comprises a light chain comprising or consisting of an amino acid sequence as set forth in SEQ ID NO:53 and a heavy chain comprising or consisting of an amino acid sequence as set forth in SEQ ID NO:52; and

(c) the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

Embodiment 59. The method, pharmaceutical composition, kit, or use of any of embodiments 1-14, wherein:

(a) the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10;

(b) the anti-human CTLA4 monoclonal antibody or antigen binding fragment thereof comprises a light chain comprising or consisting of an amino acid sequence as set forth in SEQ ID NO:53 and a heavy chain comprising or consisting of an amino acid sequence as set forth in SEQ ID NO:52; and

(c) the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

Embodiment 60. The method of any of embodiments 1-59, wherein the anti-human PD-1 monoclonal antibody is pembrolizumab, and 200 mg pembrolizumab is administered once every three weeks.
Embodiment 61. The method of any of embodiments 1-59, wherein the anti-human PD-1 monoclonal antibody is pembrolizumab, and the subject is administered 400 mg pembrolizumab, and wherein pembrolizumab is administered once every six weeks.
Embodiment 62. The method, pharmaceutical composition, kit, or use of any of embodiments 1-61, wherein 200 mg of the anti-human TIGIT monoclonal antibody or antigen binding fragment thereof is administered.
Embodiment 63. The method, pharmaceutical composition, kit, or use of any of embodiments 1-62, wherein 25 mg of the anti-human CTLA4 monoclonal antibody or antigen binding fragment thereof is administered.
Embodiment 64. The method, pharmaceutical composition, kit, or use of any of embodiments 1-63, wherein the anti-human TIGIT monoclonal antibody or antigen binding fragment thereof is administered every three weeks.
Embodiment 65. The method, pharmaceutical composition, kit, or use of any of embodiments 1-64, wherein the anti-human CTLA4 monoclonal antibody or antigen binding fragment thereof is administered every six weeks.
Embodiment 66. The method, pharmaceutical composition, kit, or use of any of embodiments 1-64, wherein 200 mg of the anti-human TIGIT monoclonal antibody or antigen binding fragment thereof is administered every three weeks.
Embodiment 67. The method, pharmaceutical composition, kit, or use of any of embodiments 1-65, wherein 25 mg of the anti-human CTLA4 monoclonal antibody or antigen binding fragment thereof is administered every six weeks.
Embodiment 68. A method of treating a cancer comprising administering to a subject in need thereof:

(a) 200 mg, 400 mg, or 2 mg/kg pembrolizumab or antigen binding fragment thereof;

(b) an effective amount of an anti-human CTLA4 antibody or antigen binding fragment thereof comprising a V_{L} region comprising an amino acid sequence as set forth in SEQ ID NO:44, and a V_{H} region comprising an amino acid sequence as set forth in SEQ ID NO:45; and

(c) an effective amount of an anti-human TIGIT antibody or antigen binding fragment thereof comprising a V_{L} region comprising an amino acid sequence as set forth in SEQ ID NO:24, and a V_{H} region comprising an amino acid sequence as set forth in SEQ ID NO:29.

Embodiment 69. A method of treating a cancer comprising administering to a subject in need thereof.

(a) 200 mg, 400 mg, or 2 mg/kg pembrolizumab or antigen binding fragment thereof;

(b) an effective amount of an anti-human CTLA4 antibody or antigen binding fragment thereof comprising a V_{L} region consisting of an amino acid sequence as set forth in SEQ ID NO:44, and a V_{H} region consisting of an amino acid sequence as set forth in SEQ ID NO:45; and

(c) an effective amount of an anti-human TIGIT antibody or antigen binding fragment thereof comprising a V_{L} region consisting of an amino acid sequence as set forth in SEQ ID NO:24, and a V_{H} region consisting of an amino acid sequence as set forth in SEQ ID NO:29.

Embodiment 70. The method of embodiment 68 or embodiment 69, wherein the pembrolizumab or antigen binding fragment thereof, the anti-CTLA4 antibody or antigen binding fragment thereof, and/or the anti-TIGIT antibody or antigen binding fragment thereof is administered via intravenous infusion.
Embodiment 71. The method of any of embodiments 68-70, wherein the anti-CTLA4 antibody is a monoclonal antibody.
Embodiment 72. The method of any of embodiments 68-71, wherein the anti-TIGIT antibody is a monoclonal antibody.
Embodiment 73. The method of any of embodiments 68-72, wherein 200 mg of pembrolizumab is administered every three weeks.
Embodiment 74. The method of any of embodiments 68-72, wherein 400 mg of pembrolizumab is administered every six weeks.
Embodiment 75. The method of any of embodiments 68-74, wherein 200 mg of the anti-human TIGIT antibody or antigen binding fragment thereof is administered every three weeks.
Embodiment 76. The method of any of embodiments 68-75, wherein 25 mg of the anti-human CTLA4 antibody or antigen binding fragment thereof is administered every six weeks.
Embodiment 77. A method of treating a cancer comprising administering to a subject in need thereof:

(a) 200 mg, 400 mg, or 2 mg/kg pembrolizumab or antigen binding fragment thereof;

(b) an effective amount of an anti-human CTLA4 antibody or antigen binding fragment thereof comprising a light chain comprising an amino acid sequence as set forth in SEQ ID NO:53 and a heavy chain comprising an amino acid sequence as set forth in SEQ ID NO:52; and

(c) an effective amount of an anti-human TIGIT antibody or antigen binding fragment thereof comprising a light chain comprising an amino acid sequence as set forth in SEQ ID NO:25 and a heavy chain comprising an amino acid sequence as set forth in SEQ ID NO:30.

Embodiment 78. A method of treating a cancer comprising administering to a subject in need thereof:

(a) 200 mg, 400 mg, or 2 mg/kg pembrolizumab or antigen binding fragment thereof;

(b) an effective amount of an anti-human CTLA4 antibody or antigen binding fragment thereof consisting of a light chain comprising an amino acid sequence as set forth in SEQ ID NO:53 and a heavy chain comprising an amino acid sequence as set forth in SEQ ID NO:52; and

(c) an effective amount an anti-human TIGIT antibody or antigen binding fragment thereof consisting of a light chain comprising an amino acid sequence as set forth in SEQ ID NO:25 and a heavy chain comprising an amino acid sequence as set forth in SEQ ID NO:30.

Embodiment 79. The method of embodiment 77 or embodiment 78, wherein the pembrolizumab or antigen binding fragment thereof, the anti-CTLA4 antibody or antigen binding fragment thereof, and/or the anti-TIGIT antibody or antigen binding fragment thereof is administered via intravenous infusion.
Embodiment 80. The method of any of embodiments 77-79, wherein the anti-CTLA4 antibody is a monoclonal antibody.
Embodiment 81. The method of any of embodiments 77-80, wherein the anti-TIGIT antibody is a monoclonal antibody.
Embodiment 82. The method of any of embodiments 77-81, wherein 200 mg of pembrolizumab is administered every three weeks.
Embodiment 83. The method of any of embodiments 77-81, wherein 400 mg of pembrolizumab is administered every six weeks.
Embodiment 84. The method of any of embodiments 77-83, wherein 200 mg of the anti-human TIGIT antibody or antigen binding fragment thereof is administered every three weeks.
Embodiment 85. The method of any of embodiments 77-84, wherein 25 mg of the anti-human CTLA4 antibody or antigen binding fragment thereof is administered every six weeks.
Embodiment 86. The method of any of embodiments 68-85 wherein the method is for treating cancer.
Embodiment 87. The method of embodiment 86, wherein the cancer comprises at least one tumor.
Embodiment 88. The method of embodiment 86 or 87, wherein the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.
Embodiment 89. The method of any of embodiments 86-88, wherein the cancer is metastatic.
Embodiment 90. The method of any of embodiments 86-89, wherein the cancer is locally advanced or resectable.
Embodiment 91. The method of any of embodiments 86-88, wherein the cancer is relapsed.
Embodiment 92. The method of any of embodiments 86-88, wherein the cancer is refractory.
Embodiment 93. The method of embodiment 92, wherein the refractory cancer (e.g., melanoma) has progressed on treatment with an anti-PD-1/L1 mAb administered either as monotherapy, or in combination with a checkpoint inhibitor or other therapy.
Embodiment 94. The method of any of embodiments 68-93, wherein the subject has received at least 2 doses of an approved anti-PD-1/L1 monoclonal antibody.
Embodiment 95. The method of any of embodiments 65-94, wherein the subject has demonstrated disease progression after PD-1/L1 treatment as defined by RECIST 1.1.
Embodiment 96. The method of any of embodiments 65-94, wherein the subject has at least one melanoma selected from the group consisting of: PD-1 refractory melanoma, first-line advanced melanoma, and neoadjuvant melanoma.
Embodiment 97. The method, pharmaceutical composition, kit, or use of any of embodiments 1-96, wherein the anti-PD-1 antibody or antigen binding fragment, the anti-TIGIT antibody or antigen binding fragment, and the anti-CTLA4 antibody or antigen binding fragment are administered contemporaneously.
Embodiment 98. The method, pharmaceutical composition, kit, or use of any of embodiments 1-96, wherein the order of administration is the anti-PD-1 antibody followed by the anti-TIGIT antibody and then the anti-CTLA4 antibody.

Cancer, infections and infectious disease are a major health concern. Cancer refers to any one of a large number of diseases characterized by the development of abnormal cells that divide uncontrollably and have the ability to infiltrate and destroy normal body tissue. Cancer survival rates are improving for many types of cancer, however further improvements in cancer screening and cancer treatment are needed, including in treatment of sarcoma, melanoma, breast cancer, pancreatic cancer, etc.

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372: 2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA® (pembrolizumab), Merck and Co., Inc., Kenilworth, N.J.; OPDIVO® (nivolumab), Bristol-Myers Squibb Company, Princeton, N.J.) and those that bind to the PD-L1 ligand (e.g., TECENTRIQ® (atezolizumab), Genentech, San Francisco, Calif.).

### PD-1 Refractory Melanoma

Analyses of clinical study data have identified 2 broad patterns of resistance to PD-1 inhibitors in patients with advanced melanoma: patients who fail to respond to PD-1 inhibitors administered as 1L therapy (also known as innate resistance), and patients who initially respond to therapy but eventually develop disease progression (also known as acquired resistance) [Pitt, J. M., et al 2016] [Restifo, N. P., et al 2016] [O'Donnell, J. S., et al 2017][Sharma, P., et al 2017].

In addition, with the approval of anti-PD-1 agents in Stage III melanoma, while the majority of patients treated with adjuvant pembrolizumab are recurrence-free at 1 year (pembrolizumab 1-year RFS is 75.4% [95% CI. 71.3 to 78.9]), some patients do not benefit from adjuvant therapy and experience disease recurrence while on anti-PD-1 therapy or shortly thereafter [Eggermont, A. M. M., et al 2018] [Weber, J., et al 2017] [Long, G. V., et al 2017].

Several mechanisms have been proposed to explain resistance to PD-1 inhibition such as insufficient antitumor T-cell generation, inadequate antitumor T-cell effector function, or impaired T-cell memory. However, these mechanisms remain largely unknown [Jenkins, R. W., et al 2018].

Clinical data in this setting is very limited. Retrospective data in melanoma patients previously exposed to anti-PD-1 inhibitors showed an ORR to available checkpoint inhibitors (CTLA-4 and CTLA-4+PD-1 combination) of 16% and 21%, respectively [Zimmer, L., et al 2017].

Phase 2 data of pembrolizumab plus ipilimumab immediately following progression on anti-PD-1 therapy in 17 evaluable melanoma patients showed an ORR of 47% and a disease control rate of 76% [Olsonm, D., et al 2018]. Recent results from the Phase 2 ILLUMINATE-204 study investigating tilsotolimod, an intratumorally delivered toll-like receptor agonist in combination with ipilimumab (Yervoy®) in 15 patients with unresectable or metastatic melanoma following failure of PD-1 inhibitor treatment, demonstrated an ORR of 47% and a disease control rate of 67% [Olsonm, D., et al 2018]. Therefore, this population represents a high medical need and effective therapies are urgently needed.

### First-Line (1L) Advanced Melanoma

Unresectable Stage III or Stage IV melanoma has historically been resistant to anticancer therapies and was associated with a median OS of less than 1 year. However, with the arrival of new therapies including targeted therapies such as BRAF and/or MEK inhibition for BRAF-mutant melanoma and immune-checkpoint-based immunotherapy (anti-CTLA-4 and anti-PD-1 inhibitors), OS for patients with metastatic melanoma has tripled [Robert, C., et al 2015] [Robert, C., et al 2015] [Robert, C., et al 2015]. The 5-year OS rate for patients treated with pembrolizumab was 38.7% and the 4-year PFS rate was 23.0% [Robert, C., et al 2019].

Despite these results, there is a large population of advanced melanoma patients (approximately 60% of all unresectable Stage III or Stage IV melanoma patients who receive anti-PD-1 inhibitors as 1L therapy for their advanced disease) who do not respond to therapy with anti-PD-1 agents.

Thus, the goal of evaluating combination strategies in this setting is to improve the overall outcome of patients with 1L advanced melanoma.

### Neoadjuvant Melanoma

The International Neoadjuvant Melanoma Consortium has recently standardized the pathologic evaluation of resected melanoma following neoadjuvant therapy including the assessment of pretreatment biopsies, initial specimen handling, histopathologic assessment of excised tumor-involved lymph nodes, and definition of pathologic response, including the definition of pCR described above [Tetzlaff, M. T., et al 2018]. The FDA has designated pCR as a surrogate endpoint to justify accelerated approval of a particular agent administered in the neoadjuvant (and adjuvant) setting for breast cancer [Center for Drug Evaluation and Research 2014] [Fumagalli, D., et al 2012].

Surgery is the main treatment for Stage I or Stage II melanoma (tumor limited to the primary site) and Stage III melanoma (tumor invading regional lymph nodes). Although the long-term RFS rates for most patients with low-risk Stage I and Stage II melanoma are excellent following surgery, patients who have high-risk features such as tumor-involved lymph nodes (Stage III) have poorer outcomes, with an average 5-year OS of approximately 50% [Gershenwald, J. E., et al. 2017]. As mentioned above, adjuvant therapy with PD-1 inhibitors pembrolizumab and nivolumab and the combination of BRAF and MEK inhibitors is standard-of-care in patients with high-risk Stage III melanoma with lymph node involvement following complete resection.

While adequate and effective surgery is the goal of early treatment of primary melanoma, some cases with bulky nodal involvement are at high risk of local and distant recurrence despite upfront surgical resection. Neoadjuvant treatment offers the benefit of an early on-treatment pathological specimen that can be profiled for additional biomarkers and correlated with survival (pathological response as a surrogate marker for RFS and OS). Neoadjuvant treatment also allows to determine the efficacy of a treatment-of-interest within an individual patient and reduce the tumor burden before surgery, facilitating the procedure. Recent preclinical data also provide support for the superior activity of T-cell checkpoint blockade when given before surgery [Liu, J., et al. 2016].

Patients with bulky resectable disease have been treated in pilot studies using a neoadjuvant approach with targeted therapy, and more recently, checkpoint inhibitors. However, improved treatments are still needed.

It has been proposed that the efficacy of anti-PD-1 or anti-PD-L1 antagonistic antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents. However, there are no clear guidelines as to which agent combined with the anti-PD-1 or anti-PD-L1 antibodies may be effective or in which patients the combination may enhance the efficacy of treatment. Thus, there is an unmet need in the art for high efficacy therapeutic combinations that can generate a robust immune response to cancer.

CTLA4 has a very close relationship with the CD28 molecule in gene structure, chromosome location, sequence homology and gene expression. Both are receptors for the co-stimulative molecule B7, mainly expressed on the surface of activated T cells. After binding to B7, CTLA4 can inhibit the activation of mouse and human T cells, playing a negative regulating role in the activation of T cells.

CTLA4 mAbs or CTLA4 ligands can prevent CTLA4 from binding to its native ligands, thereby blocking the transduction of the T cell negative regulating signal by CTLA4 and enhancing the responsiveness of T cells to various antigens. In this aspect, results from in vivo and in vitro studies are substantially in concert. At present, there are some CTLA4 mAbs being tested in clinical trials for treating prostate cancer, bladder cancer, colorectal cancer, cancer of gastrointestinal tract, liver cancer, malignant melanoma, etc. (Grosso et al., CTLA-4 blockade in tumor models: an overview of preclinical and translational research. Cancer Immun. 13:5 (2013)).

As important factors affecting the function of T cells, CTLA4 and CTLA4 mAbs can produce specific therapeutic effects on diseases by interfering with the immune microenvironment in the body. They have high efficacy and remedy the deficiency of traditional medication, opening a novel pathway of gene therapy. CTLA4 and CTLA4 mAbs are being tested in experiments and various stages of clinical trials. For example, in autoimmune diseases, they have been shown to effectively inhibit airway hyperresponsiveness in an animal model of asthma, prevent the development of rheumatic diseases, mediate immune tolerance to an allograft in the body, and the like. On the other hand, although biological gene therapy has not shown any adverse effect in short term clinical trials, attention should be paid to the potential effect after long term application. For example, excessive blockade of CTLA4-B7 signaling by CTLA4 mAbs may result in the development of autoimmune diseases. As antibodies can specifically bind to their antigens and induce the lysis of target cells or block the progress of pathology, development and utilization of drugs based on antibodies, especially humanized antibodies have important significance in the clinical treatment of malignant tumors and other immune diseases in humans.

TIGIT is an immunomodulatory receptor expressed primarily on activated T cells and NK cells (Yu et al., Nat Immunol, 10(1):48-57 (2009)). TIGIT forms part of a co-stimulatory network that consists of positive (such as CD226) and negative (such as TIGIT) immunomodulatory receptors on T cells, and ligands expressed on antigen-presenting cells (such as CD155 and CD112). Ligation of TIGIT by its ligands CD155 and CD112 expressed on tumor cells or tumor-associated macrophages may contribute to the suppression of T cell receptor signaling and T cell activation, which is essential for mounting effective anti-tumor immunity.

The data described above show that a combination of an ant-PD-1 antibody or antigen binding fragment thereof, an anti-TIGIT antibody or antigen binding fragment thereof, and an anti-CTLA4 antibody or antigen binding fragment thereof is a promising and effective treatment for treatment of a cancer (e.g., melanoma), an infection and/or an infectious disease.

Developing therapeutic agents for treating cancer encompasses using different experiments and models, e.g., ex vivo and in vivo models. Different human and murine cell lines have been used and can be purchased from various sources for these models. Murine tumor models (e.g., EMT6 and MBT-2) are described herein and were used to show the efficacy of the claimed combination treatment.

The EMT6 cell line was established from a transplantable murine mammary carcinoma that arose in a BALB/cCRGL mouse after implantation of a hyperplastic mammary alveolar nodule. The resulting tumor line (named KHJJ) was propagated in BALB/cKa mice and adapted to tissue culture after the 25th animal passage, and the cell line was named EMT. EMT6 is a clonal isolate of EMT isolated in 1971 at Stanford University. See Rockwell et al., 1977, Lab. Anim. Sci. 27: 831-851. PubMed: 338981 and Palom et al., 2000, Chem. Res. Toxicol. 13: 479-488. PubMed: 10858321.

MBT-2 is a poorly differentiated murine bladder cancer cell line derived from a transplantable N-[4-(5-nitro-2-furyl)-2-thiazolyl] formamide-induced bladder cancer in a female C3H/He mouse. The mouse bladder carcinoma cell line MBT-2 (originally described by Soloway et al. 1977 Cancer Res., 37: 2918-2929, and Soloway et al., 1980 Cancer (Phila.), 46: 1158-1158), is distributed and used by many research groups. This cell line which can be orthotopically transplanted in syngeneic C3H/He mice2 has been and is still used to study various aspects of bladder cancer. Warner et al. Neoplasia 2008; 10: 131-9.

MB49 is mouse urothelial carcinoma cell line that is widely used as an in vitro and in vivo models of bladder cancer. MB49 cells derived from an adult C57BL/6 mouse by exposure of primary bladder epithelial cell explants to 7,12-dimethylbenz[a]anthracene (DMBA) for 24 hr followed by long-term culture. See Alberto et al., 2019 Oncol Lett; 17(3): 3141-3150; and Summerhayes et al., 1979 J Natl Cancer Inst; 62:1017-1023. The MB49 cell line is used to demonstrate the effects of ageing on neoplastic transformation in long term primary cultures of the bladder. See Chan et al., 2009, BJU Int.; 104:1286-1291. doi: 10.1111/j.1464-410X.2009.08577. There is a high age-associated bladder tumour incidence in men.

### 3. Anti-Human PD-1 Monoclonal Antibodies and Antigen Binding Fragments Thereof

Provided herein are anti-human PD-1 monoclonal antibodies or antigen binding fragments thereof that can be used in the various methods, pharmaceutical compositions, kits, and uses disclosed herein. Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

In certain embodiments of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively.

In some embodiments of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:4, and a V_{H} region comprising an amino acid sequence as set forth in SEQ ID NO:9.

In other embodiments of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

In certain embodiments of various methods, pharmaceutical compositions, kits, or uses provided herein, the anti-human PD-1 monoclonal antibody or antigen binding fragment thereof comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively.

In some embodiments of various methods, pharmaceutical compositions, kits, or uses provided herein, the anti-human PD-1 monoclonal antibody or antigen binding fragment thereof comprises a V_{L} region comprising an amino acid sequence as set forth in SEQ ID NO:34, and a V_{H} region comprising an amino acid sequence as set forth in SEQ ID NO:39.

In other embodiments of various methods, pharmaceutical compositions, kits, or uses provided herein, the anti-human PD-1 monoclonal antibody or antigen binding fragment thereof comprises a light chain comprising or consisting of an amino acid sequence as set forth in SEQ ID NO:35 and a heavy chain comprising or consisting of an amino acid sequence as set forth in SEQ ID NO:40.

In various embodiments, the anti-human PD-1 monoclonal antibody or antigen binding fragment thereof comprises a variant of the amino acid sequences disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human PD-1 monoclonal antibody or antigen binding fragment thereof has a V_{L} domain with at least 95%, 90%, 85%, 80%, 75% or 50% sequence homology to one of the V_{L} domains described herein, and exhibits specific binding to PD-1. In another embodiment, the anti-human PD-1 monoclonal antibody or antigen binding fragment thereof has a V_{H} domain with at least 95%, 90%, 85%, 80%, 75% or 50% sequence homology to one of the V_{H} domains described herein, and exhibits specific binding to PD-1. In yet another embodiment, the anti-human PD-1 monoclonal antibody or antigen binding fragment thereof has a V_{L} domain with at least 95%, 90%, 85%, 80%, 75% or 50% sequence homology to one of the V_{L} domains described herein and a V_{H} domain with at least 95%, 90%, 85%, 80%, 75% or 50% sequence homology to one of the V_{H} domains described herein, and exhibits specific binding to PD-1.

In one embodiment, the anti-human PD-1 monoclonal antibody or antigen binding fragment thereof has a V_{L} domain having up to 1, 2, 3, 4, 5 or more amino acid substitutions, deletions and/or additions in one of the V_{L} domains described herein, and exhibits specific binding to PD-1. In another embodiment, the anti-human PD-1 monoclonal antibody or antigen binding fragment thereof has a V_{H} domain having up to 1, 2, 3, 4, 5 or more amino acid substitutions, deletions, and/or additions in one of the V_{H} domains described herein, and exhibits specific binding to PD-1. In yet another embodiment, the anti-human PD-1 monoclonal antibody or antigen binding fragment thereof has a V_{L} domain having up to 1, 2, 3, 4, 5 or more amino acid substitutions, deletions, and/or additions in one of the V_{L} domains described herein and a V_{H} domain having up to 1, 2, 3, 4, 5 or more amino acid substitutions, deletions, and/or additions in one of the V_{H} domains described herein, and exhibits specific binding to PD-1.

In various embodiments, the anti-human PD-1 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-PD-1 monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human PD-1 monoclonal antibody is a human antibody. In other embodiments, the anti-human PD-1 monoclonal antibody is a humanized antibody.

In some embodiments, the heavy chain of the anti-human PD-1 monoclonal antibody has a human IgG1 backbone. In other embodiments, the heavy chain of the anti-human PD-1 monoclonal antibody has a human IgG2 backbone. In yet other embodiments, the heavy chain of the anti-human PD-1 monoclonal antibody has a human IgG3 backbone. In still other embodiments, the heavy chain of the anti-human PD-1 monoclonal antibody has a human IgG4 backbone.

In some embodiments, the heavy chain of the anti-human PD-1 monoclonal antibody has a human IgG1 variant backbone. In other embodiments, the heavy chain of the anti-human PD-1 monoclonal antibody has a human IgG2 variant backbone. In yet other embodiments, the heavy chain of the anti-human PD-1 monoclonal antibody has a human IgG3 variant backbone. In still other embodiments, the heavy chain of the anti-human PD-1 monoclonal antibody has a human IgG4 variant (e.g., IgG4 with S228P mutation) backbone.

In certain embodiments, the anti-human PD-1 monoclonal antibody is selected from the group consisting of pembrolizumab, nivolumab, cemiplimab, sintilimab, tislelizumab, camrelizumab, toripalimab, pidilizumab (U.S. Pat. No. 7,332,582), AMP-514 (MedImmune LLC, Gaithersburg, Md.), PDR001 (U.S. Pat. No. 9,683,048), BGB-A317 (U.S. Pat. No. 8,735,553), and MGA012 (MacroGenics, Rockville, Md.). In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, the anti-human PD-1 monoclonal antibody can be any antibody, antigen binding fragment thereof, or variant thereof disclosed in U.S. Pat. Nos. 7,488,802, 7,521,051, 8,008,449, 8,354,509, 8,168,757, international patent publication WO/2004/004771, international patent publication WO/2004/072286, international patent publication WO/2004/056875, US patent number 2011/0271358, and international patent publication WO/2008/156712, the disclosures of which are incorporated by reference herein in their entireties.

### 4. Anti-human CTLA4 Monoclonal Antibodies and Antigen Binding Fragments Thereof

Also provided herein are anti-human CTLA4 monoclonal antibodies or antigen binding fragments thereof that can be used in the various methods, pharmaceutical compositions, kits, and uses disclosed herein. Any monoclonal antibodies that bind to a CTLA4 polypeptide, a CTLA4 polypeptide fragment, a CTLA4 peptide, or a CTLA4 epitope and block the interaction between CTLA4 and its ligand Class II MHC can be used.

In various embodiments, the anti-human CTLA4 monoclonal antibody or antigen binding fragment thereof comprises a variant of the amino acid sequences disclosed herein, for example in a claim or a Table 16 herein (e.g., at least one amino acid sequence of SEQ ID NOs: 11-20, 41-57, and 105-110). A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human CTLA4 monoclonal antibody or antigen binding fragment thereof has a V_{L} domain with at least 95%, 90%, 85%, 80%, 75% or 50% sequence homology to one of the V_{L} domains described herein (e.g., Table 16), and exhibits specific binding to CTLA4. In another embodiment, the anti-human CTLA4 monoclonal antibody or antigen binding fragment thereof has a V_{H} domain with at least 95%, 90%, 85%, 80%, 75% or 50% sequence homology to one of the V_{H} domains described herein (e.g., Table 16), and exhibits specific binding to CTLA4. In yet another embodiment, the anti-human CTLA4 monoclonal antibody or antigen binding fragment thereof has a V_{L} domain with at least 95%, 90%, 85%, 80%, 75% or 50% sequence homology to one of the V_{L} domains described herein (e.g., Table 16) and a V_{H} domain with at least 95%, 90%, 85%, 80%, 75% or 50% sequence homology to one of the V_{H} domains described herein, and exhibits specific binding to CTLA4.

In one embodiment, the anti-human CTLA4 monoclonal antibody or antigen binding fragment thereof has a V_{L} domain having up to 1, 2, 3, 4, 5 or more amino acid substitutions, deletions and/or additions in one of the V_{L} domains described herein (e.g., Table 16), and exhibits specific binding to CTLA4. In another embodiment, the anti-human CTLA4 monoclonal antibody or antigen binding fragment thereof has a V_{H} domain having up to 1, 2, 3, 4, or more amino acid substitutions, deletions, and/or additions in one of the V_{H} domains described herein (e.g., Table 16), and exhibits specific binding to CTLA4. In yet another embodiment, the anti-human CTLA4 monoclonal antibody or antigen binding fragment thereof has a V_{L} domain having up to 1, 2, 3, 4, 5 or more amino acid substitutions, deletions, and/or additions in one of the V_{L} domains described herein (e.g., Table 16) and a V_{H} domain having up to 1, 2, 3, 4, 5 or more amino acid substitutions, deletions, and/or additions in one of the V_{H} domains described herein (e.g., Table 16), and exhibits specific binding to CTLA4.

In various embodiments, the anti-human CTLA4 monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-CTLA4 monoclonal antibodies and antigen binding fragments thereof disclosed herein (e.g., Table 16) will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human CTLA4 monoclonal antibody is a human antibody. In other embodiments, the anti-human CTLA4 monoclonal antibody is a humanized antibody.

In some embodiments, the heavy chain of the anti-human CTLA4 monoclonal antibody has a human IgG1 backbone. In other embodiments, the heavy chain of the anti-human CTLA4 monoclonal antibody has a human IgG2 backbone. In yet other embodiments, the heavy chain of the anti-human CTLA4 monoclonal antibody has a human IgG3 backbone. In still other embodiments, the heavy chain of the anti-human CTLA4 monoclonal antibody has a human IgG4 backbone.

In some embodiments, the heavy chain of the anti-human CTLA4 monoclonal antibody has a human IgG1 variant backbone. In other embodiments, the heavy chain of the anti-human CTLA4 monoclonal antibody has a human IgG2 variant backbone. In yet other embodiments, the heavy chain of the anti-human CTLA4 monoclonal antibody has a human IgG3 variant backbone. In still other embodiments, the heavy chain of the anti-human CTLA4 monoclonal antibody has a human IgG4 variant (e.g., IgG4 with S228P mutation) backbone.

### 5. Anti-Human TIGIT Monoclonal Antibodies and Antigen Binding Fragments Thereof

In addition, provided herein are anti-human TIGIT monoclonal antibodies or antigen binding fragments thereof that can be used in the various methods, pharmaceutical compositions, kits, and uses disclosed herein (e.g., Table 16). Any monoclonal antibodies that bind to a TIGIT polypeptide, a TIGIT polypeptide fragment, a TIGIT peptide, or a TIGIT epitope and block the interaction between TIGIT and its ligand CD155 and/or CD112 can be used. In some embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD155. In other embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD112. In yet other embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD155 and the interaction between TIGIT and CD112.

In certain embodiments of various methods, pharmaceutical compositions, kits, or uses provided herein, the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In some embodiments of various methods, pharmaceutical compositions, kits, or uses provided herein, the anti-human TIGIT monoclonal antibody or antigen binding fragment thereof comprises a V_{L} region comprising an amino acid sequence as set forth in SEQ ID NO:24, and a V_{H} region comprising an amino acid sequence as set forth in SEQ ID NO:29.

In other embodiments of various methods, pharmaceutical compositions, kits, or uses provided herein, the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

In certain embodiments of various methods, pharmaceutical compositions, kits, or uses provided herein, the anti-human TIGIT monoclonal antibody or antigen binding fragment thereof comprises a V_{L} CDR1 comprising the amino acid sequence as set forth in SEQ ID NO:21, a V_{L} CDR2 comprising the amino acid sequence as set forth in one of SEQ ID NOS:22 or 92-104, a V_{L} CDR3 comprising the amino acid sequence as set forth in SEQ ID NO:23, and a V_{H} CDR1 comprising the amino acid sequence as set forth in SEQ ID NO:26, a V_{H} CDR2 comprising the amino acid sequence as set forth in one of SEQ ID NOS:27 or 58-76, a V_{H} CDR3 comprising the amino acid sequence as set forth in one of SEQ ID NOS:28 or 77-91.

In certain embodiments of various methods, pharmaceutical compositions, kits, or uses provided herein, the anti-human TIGIT monoclonal antibody or antigen binding fragment thereof comprises a V_{L} CDR1 comprising the amino acid sequence as set forth in SEQ ID NO:21; a V_{L} CDR2 comprising the amino acid sequence as set forth in SEQ ID NO:104 (X₁X₂KTLAE), wherein X₁ is N, A, V, W, S, T, R, H, G, I, or V, and wherein X₂ is A, N, I, L, T, or V; a V_{L} CDR3 comprising the amino acid sequence as set forth in SEQ ID NO:23; a V_{H} CDR1 comprising the amino acid sequence as set forth in SEQ ID NO:26; a V_{H} CDR2 comprising the amino acid sequence as set forth in SEQ ID NO:76 (YIDPYNX₇X₈AKYX₁₂X₁₃KFX₁₆G), wherein X₇ is D, R, L, K, F, S, Y or V, wherein X₈ is G, R, N, Q, E, L K, S, Y or V, wherein X₁₂ is N, A or S, wherein X₁₃ is E or Q, and wherein X₁i is K or Q; and a V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods, pharmaceutical compositions, kits, or uses provided herein, the anti-human TIGIT monoclonal antibody or antigen binding fragment thereof comprises a V_{L} that comprises a V_{L} CDR1, a V_{L} CDR2, and a V_{L} CDR3, and a V_{H} that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in one of SEQ ID NOS:22 or 92-104; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in one of SEQ ID NOS:27 or 58-76; the V_{H} CDR3 comprising the amino acid sequence as set forth in one of SEQ ID NOS:28 or 77-91.

In certain embodiments of various methods, pharmaceutical compositions, kits, or uses provided herein, the anti-human TIGIT monoclonal antibody or antigen binding fragment thereof comprises a V_{L} that comprises a V_{L} CDR1, a V_{L} CDR2, and a V_{L} CDR3, and a V_{H} that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in SEQ ID NO:104 (X₁X₂KTLAE), wherein X₁ is N, A, V, W, S, T, R, H, G, I, or V, and wherein X₂ is A, N, I, L, T, or V; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in SEQ ID NO:76 (YIDPYNX₇X₈AKYX₁₂X₁₃KFX₁₆G), wherein X₇ is D, R, L, K, F, S, Y or V, wherein X₈ is G, R, N, Q, E, L K, S, Y or V, wherein X₁₂ is N, A or S, wherein X₁₃ is E or Q, and wherein X₁₆ is K or Q; and the V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods, pharmaceutical compositions, kits, or uses provided herein, the anti-human TIGIT monoclonal antibody or antigen binding fragment thereof comprises a light chain that comprises a V_{L} CDR1, a V_{L} CDR2, and a V_{L} CDR3, and a heavy chain that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in one of SEQ ID NOS:22 or 92-104; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in one of SEQ ID NOS:27 or 58-76; the V_{H} CDR3 comprising the amino acid sequence as set forth in one of SEQ ID NOS:28 or 77-91.

In certain embodiments of various methods, pharmaceutical compositions, kits, or uses provided herein, the anti-human TIGIT monoclonal antibody or antigen binding fragment thereof comprises a light chain that comprises a V_{L} CDR1, a V_{L} CDR2, and a V_{L} CDR3, and a heavy chain that comprises a V_{H} CDR1, a V_{H} CDR2, and a V_{H} CDR3; the V_{L} CDR1 comprising the amino acid sequence as set forth in SEQ ID NO:21; the V_{L} CDR2 comprising the amino acid sequence as set forth in SEQ ID NO:104 (X₁X₂KTLAE), wherein X₁ is N, A, V, W, S, T, R, H, G, I, or V, and wherein X₂ is A, N, I, L, T, or V; the V_{L} CDR3 comprising the amino acid sequence as set forth in SEQ ID NO:23; the V_{H} CDR1 comprising the amino acid sequence as set forth in SEQ ID NO:26; the V_{H} CDR2 comprising the amino acid sequence as set forth in SEQ ID NO:76 (YIDPYNX₇X₈AKYX₁₂X₁₃KFX₁₆G), wherein X₇ is D, R, L, K, F, S, Y or V, wherein X₈ is G, R, N, Q, E, L K, S, Y or V, wherein X₁₂ is N, A or S, wherein X₁₃ is E or Q, and wherein X₁₆ is K or Q; and the V_{H} CDR3 comprising the amino acid sequence as set forth in SEQ ID NO:91 (GGPYGX₆YFDV), wherein X₆ is W, A, D, E, F, G, I, K, N, Q, R, S, T, V or Y.

In certain embodiments of various methods, pharmaceutical compositions, kits, or uses provided herein, the V_{H} CDR2 of the anti-human TIGIT monoclonal antibody or antigen binding fragment thereof comprises the amino acid sequence as set forth in SEQ ID NO:58. In some embodiments, the V_{H} CDR2 of the anti-human TIGIT monoclonal antibody or antigen binding fragment thereof comprises the amino acid sequence as set forth in SEQ ID NO:59. In other embodiments, the V_{H} CDR2 of the anti-human TIGIT monoclonal antibody or antigen binding fragment thereof comprises the amino acid sequence as set forth in SEQ ID NO:60. In yet other embodiments, the V_{H} CDR2 of the anti-human TIGIT monoclonal antibody or antigen binding fragment thereof comprises the amino acid sequence as set forth in SEQ ID NO:61. In certain embodiments, the V_{H} CDR2 of the anti-human TIGIT monoclonal antibody or antigen binding fragment thereof comprises the amino acid sequence as set forth in SEQ ID NO:62. In some embodiments, the V_{H} CDR2 of the anti-human TIGIT monoclonal antibody or antigen binding fragment thereof comprises the amino acid sequence as set forth in SEQ ID NO:63. In other embodiments, the V_{H} CDR2 of the anti-human TIGIT monoclonal antibody or antigen binding fragment thereof comprises the amino acid sequence as set forth in SEQ ID NO:64. In yet other embodiments, the V_{H} CDR2 of the anti-human TIGIT monoclonal antibody or antigen binding fragment thereof comprises the amino acid sequence as set forth in SEQ ID NO:65. In certain embodiments, the V_{H} CDR2 of the anti-human TIGIT monoclonal antibody or antigen binding fragment thereof comprises the amino acid sequence as set forth in SEQ ID NO:66. In some embodiments, the V_{H} CDR2 of the anti-human TIGIT monoclonal antibody or antigen binding fragment thereof comprises the amino acid sequence as set forth in SEQ ID NO:67. In other embodiments, the V_{H} CDR2 of the anti-human TIGIT monoclonal antibody or antigen binding fragment thereof comprises the amino acid sequence as set forth in SEQ ID NO:68. In yet other embodiments, the V_{H} CDR2 of the anti-human TIGIT monoclonal antibody or antigen binding fragment thereof comprises the amino acid sequence as set forth in SEQ ID NO:69. In certain embodiments, the V_{H} CDR2 of the anti-human TIGIT monoclonal antibody or antigen binding fragment thereof comprises the amino acid sequence as set forth in SEQ ID NO:70. In some embodiments, the V_{H} CDR2 of the anti-human TIGIT monoclonal antibody or antigen binding fragment thereof comprises the amino acid sequence as set forth in SEQ ID NO:71. In other embodiments, the V_{H} CDR2 of the anti-human TIGIT monoclonal antibody or antigen binding fragment thereof comprises the amino acid sequence as set forth in SEQ ID NO:72. In yet other embodiments, the V_{H} CDR2 of the anti-human TIGIT monoclonal antibody or antigen binding fragment thereof comprises the amino acid sequence as set forth in SEQ ID NO:73. In certain embodiments, the V_{H} CDR2 of the anti-human TIGIT monoclonal antibody or antigen binding fragment thereof comprises the amino acid sequence as set forth in SEQ ID NO:74. In some embodiments, the V_{H} CDR2 of the anti-human TIGIT monoclonal antibody or antigen binding fragment thereof comprises the amino acid sequence as set forth in SEQ ID NO:75.

In certain embodiments of various methods, pharmaceutical compositions, kits, or uses provided herein, the V_{H} CDR3 of the anti-human TIGIT monoclonal antibody or antigen binding fragment thereof comprises the amino acid sequence as set forth in SEQ ID NO:77. In some embodiments, the V_{H} CDR3 of the anti-human TIGIT monoclonal antibody or antigen binding fragment thereof comprises the amino acid sequence as set forth in SEQ ID NO:78. In other embodiments, the V_{H} CDR3 of the anti-human TIGIT monoclonal antibody or antigen binding fragment thereof comprises the amino acid sequence as set forth in SEQ ID NO:79. In yet other embodiments, the V_{H} CDR3 of the anti-human TIGIT monoclonal antibody or antigen binding fragment thereof comprises the amino acid sequence as set forth in SEQ ID NO:80. In certain embodiments, the V_{H} CDR3 of the anti-human TIGIT monoclonal antibody or antigen binding fragment thereof comprises the amino acid sequence as set forth in SEQ ID NO:81. In some embodiments, the V_{H} CDR3 of the anti-human TIGIT monoclonal antibody or antigen binding fragment thereof comprises the amino acid sequence as set forth in SEQ ID NO:82. In other embodiments, the V_{H} CDR3 of the anti-human TIGIT monoclonal antibody or antigen binding fragment thereof comprises the amino acid sequence as set forth in SEQ ID NO:83. In yet other embodiments, the V_{H} CDR3 of the anti-human TIGIT monoclonal antibody or antigen binding fragment thereof comprises the amino acid sequence as set forth in SEQ ID NO:84. In certain embodiments, the V_{H} CDR3 of the anti-human TIGIT monoclonal antibody or antigen binding fragment thereof comprises the amino acid sequence as set forth in SEQ ID NO:85. In some embodiments, the V_{H} CDR3 of the anti-human TIGIT monoclonal antibody or antigen binding fragment thereof comprises the amino acid sequence as set forth in SEQ ID NO:86. In other embodiments, the V_{H} CDR3 of the anti-human TIGIT monoclonal antibody or antigen binding fragment thereof comprises the amino acid sequence as set forth in SEQ ID NO:87. In yet other embodiments, the V_{H} CDR3 of the anti-human TIGIT monoclonal antibody or antigen binding fragment thereof comprises the amino acid sequence as set forth in SEQ ID NO:88. In certain embodiments, the V_{H} CDR3 of the anti-human TIGIT monoclonal antibody or antigen binding fragment thereof comprises the amino acid sequence as set forth in SEQ ID NO:89. In some embodiments, the V_{H} CDR3 of the anti-human TIGIT monoclonal antibody or antigen binding fragment thereof comprises the amino acid sequence as set forth in SEQ ID NO:90.

In certain embodiments of various methods, pharmaceutical compositions, kits, or uses provided herein, the V_{L} CDR2 of the anti-human TIGIT monoclonal antibody or antigen binding fragment thereof comprises the amino acid sequence as set forth in SEQ ID NO:92. In some embodiments, the V_{L} CDR2 of the anti-human TIGIT monoclonal antibody or antigen binding fragment thereof comprises the amino acid sequence as set forth in SEQ ID NO:93. In other embodiments, the V_{L} CDR2 of the anti-human TIGIT monoclonal antibody or antigen binding fragment thereof comprises the amino acid sequence as set forth in SEQ ID NO:94. In yet other embodiments, the V_{L} CDR2 of the anti-human TIGIT monoclonal antibody or antigen binding fragment thereof comprises the amino acid sequence as set forth in SEQ ID NO:95. In certain embodiments, the V_{L} CDR2 of the anti-human TIGIT monoclonal antibody or antigen binding fragment thereof comprises the amino acid sequence as set forth in SEQ ID NO:96. In some embodiments, the V_{L} CDR2 of the anti-human TIGIT monoclonal antibody or antigen binding fragment thereof comprises the amino acid sequence as set forth in SEQ ID NO:97. In other embodiments, the V_{L} CDR2 of the anti-human TIGIT monoclonal antibody or antigen binding fragment thereof comprises the amino acid sequence as set forth in SEQ ID NO:98. In yet other embodiments, the V_{L} CDR2 of the anti-human TIGIT monoclonal antibody or antigen binding fragment thereof comprises the amino acid sequence as set forth in SEQ ID NO:99. In certain embodiments, the V_{L} CDR2 of the anti-human TIGIT monoclonal antibody or antigen binding fragment thereof comprises the amino acid sequence as set forth in SEQ ID NO:100. In some embodiments, the V_{L} CDR2 of the anti-human TIGIT monoclonal antibody or antigen binding fragment thereof comprises the amino acid sequence as set forth in SEQ ID NO:101. In other embodiments, the V_{L} CDR2 of the anti-human TIGIT monoclonal antibody or antigen binding fragment thereof comprises the amino acid sequence as set forth in SEQ ID NO:102. In yet other embodiments, the V_{L} CDR2 of the anti-human TIGIT monoclonal antibody or antigen binding fragment thereof comprises the amino acid sequence as set forth in SEQ ID NO:103.

In various embodiments, the anti-human TIGIT monoclonal antibody or antigen binding fragment thereof comprises a variant of the amino acid sequences disclosed herein. A variant amino acid sequence is identical to the reference sequence except having one, two, three, four, or five amino acid substitutions, deletions, and/or additions. In some embodiments, the substitutions, deletions and/or additions are in the CDRs. In some embodiments, the substitutions, deletions and/or additions are in the framework regions. In certain embodiments, the one, two, three, four, or five of the amino acid substitutions are conservative substitutions.

In one embodiment, the anti-human TIGIT monoclonal antibody or antigen binding fragment thereof has a V_{L} domain with at least 95%, 90%, 85%, 80%, 75% or 50% sequence homology to one of the V_{L} domains described herein (e.g., Table 16), and exhibits specific binding to TIGIT. In another embodiment, the anti-human TIGIT monoclonal antibody or antigen binding fragment thereof has a V_{H} domain with at least 95%, 90%, 85%, 80%, 75% or 50% sequence homology to one of the V_{H} domains described herein (e.g., Table 16), and exhibits specific binding to TIGIT. In yet another embodiment, the anti-human TIGIT monoclonal antibody or antigen binding fragment thereof has a V_{L} domain with at least 95%, 90%, 85%, 80%, 75% or 50% sequence homology to one of the V_{L} domains described herein and a V_{H} domain with at least 95%, 90%, 85%, 80%, 75% or 50% sequence homology to one of the V_{H} domains described herein (e.g., Table 16), and exhibits specific binding to TIGIT.

In one embodiment, the anti-human TIGIT monoclonal antibody or antigen binding fragment thereof has a V_{L} domain having up to 1, 2, 3, 4, 5 or more amino acid substitutions, deletions and/or additions in one of the V_{L} domains described herein (e.g., Table 16), and exhibits specific binding to TIGIT. In another embodiment, the anti-human TIGIT monoclonal antibody or antigen binding fragment thereof has a V_{H} domain having up to 1, 2, 3, 4, or more amino acid substitutions, deletions, and/or additions in one of the V_{H} domains described herein, and exhibits specific binding to TIGIT. In yet another embodiment, the anti-human TIGIT monoclonal antibody or antigen binding fragment thereof has a V_{L} domain having up to 1, 2, 3, 4, or more amino acid substitutions, deletions, and/or additions in one of the V_{L} domains described herein and a V_{H} domain having up to 1, 2, 3, 4, 5 or more amino acid substitutions, deletions, and/or additions in one of the V_{H} domains described herein (e.g., Table 16), and exhibits specific binding to TIGIT.

In various embodiments, the anti-human TIGIT monoclonal antibody or antigen binding fragment thereof is selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances, an IgG4 constant domain, for example, may be used. In various embodiments, the heavy chain constant domain contains one or more amino acid mutations (e.g., IgG4 with S228P mutation) to generate desired characteristics of the antibody. These desired characteristics include but are not limited to modified effector functions, physical or chemical stability, half-life of antibody, etc.

Ordinarily, amino acid sequence variants of the anti-TIGIT monoclonal antibodies and antigen binding fragments thereof disclosed herein will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference sequence, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3. M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

In some embodiments, the anti-human TIGIT monoclonal antibody is a human antibody. In other embodiments, the anti-human TIGIT monoclonal antibody is a humanized antibody.

In some embodiments, the heavy chain of the anti-human TIGIT monoclonal antibody has a human IgG1 backbone. In other embodiments, the heavy chain of the anti-human TIGIT monoclonal antibody has a human IgG2 backbone. In yet other embodiments, the heavy chain of the anti-human TIGIT monoclonal antibody has a human IgG3 backbone. In still other embodiments, the heavy chain of the anti-human TIGIT monoclonal antibody has a human IgG4 backbone.

In some embodiments, the heavy chain of the anti-human TIGIT monoclonal antibody has a human IgG1 variant backbone. In other embodiments, the heavy chain of the anti-human TIGIT monoclonal antibody has a human IgG2 variant backbone. In yet other embodiments, the heavy chain of the anti-human TIGIT monoclonal antibody has a human IgG3 variant backbone. In still other embodiments, the heavy chain of the anti-human TIGIT monoclonal antibody has a human IgG4 variant (e.g., IgG4 with S228P mutation) backbone.

In certain embodiments, the anti-human TIGIT monoclonal antibody is selected from the group consisting of BMS-986207, OMP-313M32, MTIG7192A (RG6058) and PTZ-201 (ASP8374). In one embodiment, the anti-human TIGIT monoclonal antibody is BMS-986207. In another embodiment, the anti-human TIGIT monoclonal antibody is OMP-313M32. In yet another embodiment, the anti-human TIGIT monoclonal antibody is MTIG7192A (RG6058). In still another embodiment, the anti-human TIGIT monoclonal antibody is PTZ-201 (ASP8374).

In some embodiments, the anti-human TIGIT monoclonal antibody can be any antibody, antigen binding fragment thereof, or variant thereof disclosed in WO 2016/028656 and WO 2017/030823, the disclosures of which are incorporated by reference herein in their entireties.

### 6. Methods of Treating Cancer or Infection Using a Combination of an Anti-PD-1 Monoclonal Antibody, an Anti-CTLA4 Monoclonal Antibody, and an Anti-TIGIT Monoclonal Antibody

In another aspect, provided herein are methods of treating cancer (e.g., colorectal cancer) or an infectious disease (e.g., a viral infection) using a combination of an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof,

In certain embodiments, provided herein are methods of treating cancer, comprising administering to a human patient in need thereof.
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., DLBCL or NHL), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In various embodiments, the cancer is locally advanced or resectable. In various embodiments, the cancer has been treated or is being treated with an adjuvant. In various embodiments, the cancer has been treated or is being treated with a neoadjuvant. In some embodiments, the cancer is relapsed. In other embodiments, the cancer is refractory. In yet other embodiments, the cancer is relapsed and refractory. For example, the cancer is a melanoma. In certain embodiments, the cancer is a treatment of PD-1 refractory melanoma.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In another embodiment, the cancer is naïve rrNHL. In yet still another embodiment, the cancer is PD-1 refractory rrNHL.

In certain embodiments, provided herein is a method of treating colorectal cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In some embodiments, provided herein is a method of treating gastric cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In other embodiments, provided herein is a method of treating head and neck cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In yet other embodiments, provided herein is a method of treating refractory head and neck cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In still other embodiments, provided herein is a method of treating lung cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In certain embodiments, provided herein is a method of treating non-small cell lung cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In some embodiments, provided herein is a method of treating breast cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In other embodiments, provided herein is a method of treating cervical cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In yet other embodiments, provided herein is a method of treating ovarian cancer, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In still other embodiments, provided herein is a method of treating DLBCL, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In certain embodiments, provided herein is a method of treating NHL, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In some embodiments, provided herein is a method of treating rrNHL, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In other embodiments, provided herein is a method of treating naïve rrNHL, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In yet other embodiments, provided herein is a method of treating PD-1 refractory rrNHL, comprising administering to a human patient in need thereof:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In certain embodiments, the method of treating cancer comprises administering to a human patient in need thereof:
(a) an anti-human PD-1 monoclonal antibody or antigen binding fragment thereof as disclosed in Table 15 or Table 16;
(b) an anti-human CTLA4 monoclonal antibody or antigen binding fragment thereof as disclosed in Table 15 or Table 16; and
(c) an anti-human TIGIT antibody or antigen binding fragment thereof as disclosed in Table 16.

Any combination of an anti-PD-1 monoclonal antibody or antigen binding fragment thereof disclosed in Table 15 or Table 16, an anti-CTLA4 monoclonal antibody or antigen binding fragment thereof disclosed in Table 15 or Table 16, and an anti-TIGIT monoclonal antibody or antigen binding fragment thereof disclosed in Table 15 or Table 16 are contemplated herein in methods of treating cancer (e.g., colorectal cancer) or an infectious disease (e.g., a viral infection, a bacterial infection, etc.).

Any combination of an anti-PD-1 monoclonal antibody or antigen binding fragment thereof disclosed in Table 16, an anti-CTLA4 monoclonal antibody or antigen binding fragment thereof disclosed in Table 16, and an anti-TIGIT monoclonal antibody or antigen binding fragment thereof disclosed in Table 16 are contemplated herein in methods of treating cancer (e.g., colorectal cancer) or an infectious disease (e.g., a viral infection, a bacterial infection, etc.).

In various embodiments of the methods, the administering of one or more of the anti-PD-1, anti-CTLA4 and anti-TIGIT is performed by injection. In various embodiments of the methods, the administering of one or more of the anti-PD-1, anti-CTLA4 and anti-TIGIT is performed by infusion.

In various embodiments of the methods, the administering of (a), (b), and (c) is performed sequentially. For example, (a) is administered, then (b) and (c). In various embodiments, (a) is administered, then (c) and (b). In various embodiments, (b) is administered, then (a) and (c). In various embodiments, (b) is administered, then (c) and (a). In various embodiments, (c) is administered, then (a) and (b). In various embodiments, (c) is administered, then (b) and (c).

Alternatively, in various embodiments of the methods, the administering of (a), (b), and (c) is performed concurrently.

### 7. Methods of Enhancing Immune Cell Activity Using a Combination of an Anti-PD-1 Monoclonal Antibody, an Anti-CTLA4 Monoclonal Antibody, and an Anti-TIGIT Monoclonal Antibody

Further, the antibodies or antigen binding fragments of disclosed herein can enhance the activity of an immune cell. The increase of the activity of an immune cell can be detected using any methods known in the art. In one embodiment, the increase in activity of an immune cell can be detected by measuring the proliferation of the immune cell. For example, an increase in activity of a T cell can be detected by measuring the proliferation of the T cell or signal transduction events such as tyrosine phosphorylation of immune receptors or downstream kinases that transmit signals to transcriptional regulators. In other embodiments, the increase in activity of an immune cell can be detected by measuring CTL or NK cell cytotoxic function on specific target cells or IFN-γ cytokine responses, which are associated with stimulation of immunity. In yet other embodiments, the increase in activity of an immune cell can be detected by measuring T cell activation ex vivo in a sample derived from the subject. In one embodiment, the increase in T cell activity is determined by measuring production of one or more cytokines selected from the group consisting of: IL-1, IL-2, IL-6, IL-12, IL-17, IL-22, IL-23, GM-CSF, IFN-γ, and TNF-α. In some embodiments, the ability of the antibodies or antigen binding fragments of the invention to increase the activity of an immune cell can be detected by CD25 and CD69 upregulation by flow cytometry.

Thus, also provided herein are methods of enhancing T cell activity (e.g., increasing cytokine production and/or cell proliferation), comprising contacting the T cells with:
(a) an anti-human PD-1 antibody or antigen binding fragment thereof,
(b) an anti-human CTLA4 antibody or antigen binding fragment thereof, and
(c) an anti-human TIGIT antibody or antigen binding fragment thereof.

In some embodiments, the enhancement of T cell activity occurs in vitro. In other embodiments, the enhancement of T cell activity occurs in vivo.

In some embodiments, the enhancement of T cell activity is measured by increased cytokine production. In other embodiments, the enhancement of T cell activity is measured by increased cell proliferation.

Thus, in certain embodiments, provided herein is a method of increasing cytokine production of T cells, comprising contacting the T cells with:
(a) an anti-human PD-1 antibody or antigen binding fragment thereof,
(b) an anti-human CTLA4 antibody or antigen binding fragment thereof, and
(c) an anti-human TIGIT antibody or antigen binding fragment thereof.

In some embodiments, the increased cytokine production of T cells occurs in vitro. In other embodiments, the increased cytokine production of T cells occurs in vivo.

In some embodiments, provided herein is a method of increasing proliferation of T cells, comprising contacting the T cells with:
(a) an anti-human PD-1 antibody or antigen binding fragment thereof,
(b) an anti-human CTLA4 antibody or antigen binding fragment thereof, and
(c) an anti-human TIGIT antibody or antigen binding fragment thereof.

In some embodiments, the increased proliferation of T cells occurs in vitro. In other embodiments, the increased proliferation of T cells occurs in vivo.

In other embodiments, provided herein is a method of increasing cytokine production and proliferation of T cells, comprising contacting the T cells with:
(a) an anti-human PD-1 antibody or antigen binding fragment thereof,
(b) an anti-human CTLA4 antibody or antigen binding fragment thereof, and
(c) an anti-human TIGIT antibody or antigen binding fragment thereof.

In some embodiments, the increased cytokine production and the increased proliferation of T cells occur in vitro. In other embodiments, the increased cytokine production and the increased proliferation of T cells occur in vivo.

In certain embodiments, the cytokine is selected from the group consisting of IL-1, IL-2, IL-6, IL-12, IL-17, IL-22, IL-23, GM-CSF, IFN-γ, and TNF-α. In one embodiment, the cytokine is IL-1. In another embodiment, the cytokine is IL-2. In yet another embodiment, the cytokine is IL-6. In still another embodiment, the cytokine is IL-12. In one embodiment, the cytokine is IL-17. In another embodiment, the cytokine is IL-22. In yet another embodiment, the cytokine is IL-23. In still another embodiment, the cytokine is GM-CSF. In one embodiment, the cytokine is IFN-γ. In another embodiment, the cytokine is TNF-α. In some embodiments, the cytokine is one, two, three, four, five, six, seven, eight, nine, or ten cytokines selected from the group consisting of IL-1, IL-2, IL-6, IL-12, IL-17, IL-22, IL-23, GM-CSF, IFN-γ, and TNF-α.

In some embodiments, the maximal enhancement of T cell activity is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In certain embodiments, the maximal increase of cytokine production is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold. In other embodiments, the maximal increase of T cell proliferation is at least about 10%, 20%, 30%, 40%, 45%, 50%, 55%, 60%, 65%, 70%, 75%, 80%, 85%, 90%, 95%, 100%, 2-fold, 3-fold, 4-fold, 5-fold, 10-fold, 15-fold, 20-fold, 50-fold, 75-fold, or 100-fold.

In some embodiments, the maximal enhancement of T cell activity is relative to the T cell activity in cells that are not contacted with a combination of an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof. In some embodiments, the maximal enhancement of T cell activity is relative to the T cell activity in cells that are not contacted with any antibody at all. In some embodiments, the maximal enhancement of T cell activity is relative to the T cell activity in cells that are contacted with an unrelated antibody (e.g., an antibody that does not specifically bind to PD-1, CTLA4, or TIGIT). In some embodiments, the maximal enhancement of T cell activity is relative to the T cell activity in cells that are contacted with one or two antibodies selected from the group consisting of an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof. In some embodiments, the maximal enhancement of T cell activity is relative to the T cell activity in cells that are contacted with an anti-PD-1 monoclonal antibody or antigen binding fragment thereof. In some embodiments, the maximal enhancement of T cell activity is relative to the T cell activity in cells that are contacted with an anti-CTLA4 monoclonal antibody or antigen binding fragment thereof. In some embodiments, the maximal enhancement of T cell activity is relative to the T cell activity in cells that are contacted with an anti-TIGIT monoclonal antibody or antigen binding fragment thereof. In some embodiments, the maximal enhancement of T cell activity is relative to the T cell activity in cells that are contacted with an anti-PD-1 monoclonal antibody or antigen binding fragment thereof and an anti-CTLA4 monoclonal antibody or antigen binding fragment thereof. In some embodiments, the maximal enhancement of T cell activity is relative to the T cell activity in cells that are contacted with an anti-PD-1 monoclonal antibody or antigen binding fragment thereof and an anti-TIGIT monoclonal antibody or antigen binding fragment thereof. In some embodiments, the maximal enhancement of T cell activity is relative to the T cell activity in cells that are contacted with an anti-CTLA4 monoclonal antibody or antigen binding fragment thereof and an anti-TIGIT monoclonal antibody or antigen binding fragment thereof.

In some embodiments, the maximal increase of cytokine production is relative to the cytokine production in cells that are not contacted with a combination of an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof. In some embodiments, the maximal increase of cytokine production is relative to the cytokine production in cells that are not contacted with any antibody at all. In some embodiments, the maximal increase of cytokine production is relative to the cytokine production in cells that are contacted with an unrelated antibody (e.g., an antibody that does not specifically bind to PD-1, CTLA4, or TIGIT). In some embodiments, the maximal increase of cytokine production is relative to the cytokine production in cells that are contacted with one or two antibodies selected from the group consisting of an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof. In some embodiments, the maximal increase of cytokine production is relative to the cytokine production in cells that are contacted with an anti-PD-1 monoclonal antibody or antigen binding fragment thereof. In some embodiments, the maximal increase of cytokine production is relative to the cytokine production in cells that are contacted with an anti-CTLA4 monoclonal antibody or antigen binding fragment thereof. In some embodiments, the maximal increase of cytokine production is relative to the cytokine production in cells that are contacted with an anti-TIGIT monoclonal antibody or antigen binding fragment thereof. In some embodiments, the maximal increase of cytokine production is relative to the cytokine production in cells that are contacted with an anti-PD-1 monoclonal antibody or antigen binding fragment thereof and an anti-CTLA4 monoclonal antibody or antigen binding fragment thereof. In some embodiments, the maximal increase of cytokine production is relative to the cytokine production in cells that are contacted with an anti-PD-1 monoclonal antibody or antigen binding fragment thereof and an anti-TIGIT monoclonal antibody or antigen binding fragment thereof. In some embodiments, the maximal increase of cytokine production is relative to the cytokine production in cells that are contacted with an anti-CTLA4 monoclonal antibody or antigen binding fragment thereof and an anti-TIGIT monoclonal antibody or antigen binding fragment thereof.

In some embodiments, the maximal increase of T cell proliferation is relative to the proliferation of T cells that are not contacted with a combination of an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof. In some embodiments, the maximal increase of T cell proliferation is relative to the proliferation of T cells that are not contacted with any antibody at all. In some embodiments, the maximal increase of T cell proliferation is relative to the proliferation of T cells that are contacted with an unrelated antibody (e.g., an antibody that does not specifically bind to PD-1, CTLA4, or TIGIT). In some embodiments, the maximal increase of T cell proliferation is relative to the proliferation of T cells that are contacted with one or two antibodies selected from the group consisting of an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof. In some embodiments, the maximal increase of T cell proliferation is relative to the proliferation of T cells that are contacted with an anti-PD-1 monoclonal antibody or antigen binding fragment thereof. In some embodiments, the maximal increase of T cell proliferation is relative to the proliferation of T cells that are contacted with an anti-CTLA4 monoclonal antibody or antigen binding fragment thereof. In some embodiments, the maximal increase of T cell proliferation is relative to the proliferation of T cells that are contacted with an anti-TIGIT monoclonal antibody or antigen binding fragment thereof. In some embodiments, the maximal increase of T cell proliferation is relative to the proliferation of T cells that are contacted with an anti-PD-1 monoclonal antibody or antigen binding fragment thereof and an anti-CTLA4 monoclonal antibody or antigen binding fragment thereof. In some embodiments, the maximal increase of T cell proliferation is relative to the proliferation of T cells that are contacted with an anti-PD-1 monoclonal antibody or antigen binding fragment thereof and an anti-TIGIT monoclonal antibody or antigen binding fragment thereof. In some embodiments, the maximal increase of T cell proliferation is relative to the proliferation of T cells that are contacted with an anti-CTLA4 monoclonal antibody or antigen binding fragment thereof and an anti-TIGIT monoclonal antibody or antigen binding fragment thereof.

In certain embodiments, the method of enhancing T cell activity comprises contacting the T cells with:
(a) an anti-human PD-1 monoclonal antibody or antigen binding fragment thereof as disclosed in Table 16;
(b) an anti-human CTLA4 monoclonal antibody or antigen binding fragment thereof as disclosed in Table 16; and
(c) an anti-human TIGIT monoclonal antibody or antigen binding fragment thereof as disclosed in Table 16.

In some embodiments, the enhancement of T cell activity occurs in vitro. In other embodiments, the enhancement of T cell activity occurs in vivo.

In certain embodiments, the method of increasing cytokine production of T cells comprises contacting the T cells with:
(a) an anti-human PD-1 monoclonal antibody or antigen binding fragment thereof as disclosed in Table 16;
(b) an anti-human CTLA4 monoclonal antibody or antigen binding fragment thereof as disclosed in Table 16; and
(c) an anti-human TIGIT monoclonal antibody or antigen binding fragment thereof as disclosed in Table 16.

In some embodiments, the increased cytokine production of T cells occurs in vitro. In other embodiments, the increased cytokine production of T cells occurs in vivo.

In certain embodiments, the method of increasing T cell proliferation comprises contacting the T cells with:
(a) an anti-human PD-1 monoclonal antibody or antigen binding fragment thereof as disclosed in Table 16;
(b) an anti-human CTLA4 monoclonal antibody or antigen binding fragment thereof as disclosed in Table 16; and
(c) an anti-human TIGIT monoclonal antibody or antigen binding fragment thereof as disclosed in Table 16.

In some embodiments, the increased proliferation of T cells occurs in vitro. In other embodiments, the increased proliferation of T cells occurs in vivo.

In one specific embodiment, the method of enhancing T cell activity comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of increasing cytokine production of T cells comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of increasing T cell proliferation comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively; (b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of enhancing T cell activity comprises contacting the T cells with: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human CTLA4 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO: 19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In another specific embodiment, the method of increasing cytokine production of T cells comprises contacting the T cells with: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human CTLA4 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In another specific embodiment, the method of increasing T cell proliferation comprises contacting the T cells with: (a) an anti-human PD-1 monoclonal antibody or antigen binding fragment thereof that comprises a V_{L} region comprising an amino acid sequence as set forth in SEQ ID NO:4, and a V_{H} region comprising an amino acid sequence as set forth in SEQ ID NO:9; (b) an anti-human CTLA4 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO: 19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of enhancing T cell activity comprises contacting the T cells with: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human CTLA4 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In yet another specific embodiment, the method of increasing cytokine production of T cells comprises contacting the T cells with: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human CTLA4 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In yet another specific embodiment, the method of increasing T cell proliferation comprises contacting the T cells with: (a) an anti-human PD-1 monoclonal antibody or antigen binding fragment thereof that comprises a light chain comprising or consisting of an amino acid sequence as set forth in SEQ ID NO:5 and a heavy chain comprising or consisting of an amino acid sequence as set forth in SEQ ID NO:10; (b) an anti-human CTLA4 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In one specific embodiment, the method of enhancing T cell activity comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of increasing cytokine production of T cells comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS: 11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In one specific embodiment, the method of increasing T cell proliferation comprises contacting the T cells with: (a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively; (b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and (c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

In another specific embodiment, the method of enhancing T cell activity comprises contacting the T cells with: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human CTLA4 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO: 19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In another specific embodiment, the method of increasing cytokine production of T cells comprises contacting the T cells with: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human CTLA4 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In another specific embodiment, the method of increasing T cell proliferation comprises contacting the T cells with: (a) an anti-human PD-1 monoclonal antibody or antigen binding fragment thereof that comprises a V_{L} region comprising an amino acid sequence as set forth in SEQ ID NO:34, and a V_{H} region comprising an amino acid sequence as set forth in SEQ ID NO:39; (b) an anti-human CTLA4 monoclonal antibody or antigen binding fragment thereof that comprises a V_{L} region comprising an amino acid sequence as set forth in SEQ ID NO:14, and a V_{H} region comprising an amino acid sequence as set forth in SEQ ID NO:19; and (c) an anti-human TIGIT monoclonal antibody or antigen binding fragment thereof that comprises a V_{L} region comprising an amino acid sequence as set forth in SEQ ID NO:24, and a V_{H} region comprising an amino acid sequence as set forth in SEQ ID NO:29.

In yet another specific embodiment, the method of enhancing T cell activity comprises contacting the T cells with: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human CTLA4 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In yet another specific embodiment, the method of increasing cytokine production of T cells comprises contacting the T cells with: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human CTLA4 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In yet another specific embodiment, the method of increasing T cell proliferation comprises contacting the T cells with: (a) an anti-human PD-1 monoclonal antibody or antigen binding fragment thereof that comprises a light chain comprising or consisting of an amino acid sequence as set forth in SEQ ID NO:35 and a heavy chain comprising or consisting of an amino acid sequence as set forth in SEQ ID NO:40; (b) an anti-human CTLA4 monoclonal antibody or antigen binding fragment thereof that comprises a light chain comprising or consisting of an amino acid sequence as set forth in SEQ ID NO:15 and a heavy chain comprising or consisting of an amino acid sequence as set forth in SEQ ID NO:20; and (c) an anti-human TIGIT monoclonal antibody or antigen binding fragment thereof that comprises a light chain comprising or consisting of an amino acid sequence as set forth in SEQ ID NO:25 and a heavy chain comprising or consisting of an amino acid sequence as set forth in SEQ ID NO:30.

In some embodiments, the anti-human PD-1 monoclonal antibody is a human antibody. In other embodiments, the anti-human PD-1 monoclonal antibody is a humanized antibody.

In some embodiments, the anti-human CTLA4 monoclonal antibody is a human antibody. In other embodiments, the anti-human CTLA4 monoclonal antibody is a humanized antibody.

In some embodiments, the anti-human TIGIT monoclonal antibody is a human antibody. In other embodiments, the anti-human TIGIT monoclonal antibody is a humanized antibody.

Thus, in certain embodiments, provided herein is a method for enhancing T cell activity, comprising contacting the T cells with:
(a) a human or humanized anti-human PD-1 monoclonal antibody or antigen binding fragment thereof,
(b) a human or humanized anti-human CTLA4 monoclonal antibody or antigen binding fragment thereof, and
(c) a human or humanized anti-human TIGIT monoclonal antibody or antigen binding fragment thereof.

In some embodiments, the enhancement of T cell activity occurs in vitro. In other embodiments, the enhancement of T cell activity occurs in vivo.

In some embodiments, provided herein is a method for increasing cytokine production of T cells, comprising contacting the T cells with:
(a) a human or humanized anti-human PD-1 monoclonal antibody or antigen binding fragment thereof,
(b) a human or humanized anti-human CTLA4 monoclonal antibody or antigen binding fragment thereof, and
(c) a human or humanized anti-human TIGIT monoclonal antibody or antigen binding fragment thereof.

In some embodiments, the increased cytokine production of T cells occurs in vitro. In other embodiments, the increased cytokine production of T cells occurs in vivo.

In other embodiments, provided herein is a method for increasing proliferation of T cells, comprising contacting the T cells with:
(a) a human or humanized anti-human PD-1 monoclonal antibody or antigen binding fragment thereof,
(b) a human or humanized anti-human CTLA4 monoclonal antibody or antigen binding fragment thereof, and
(c) a human or humanized anti-human TIGIT monoclonal antibody or antigen binding fragment thereof.

In some embodiments, the increased proliferation of T cells occurs in vitro. In other embodiments, the increased proliferation of T cells occurs in vivo.

Any combination of an anti-PD-1 monoclonal antibody or antigen binding fragment thereof disclosed in Table 16, an anti-CTLA4 monoclonal antibody or antigen binding fragment thereof disclosed in Table 16, and an anti-TIGIT monoclonal antibody or antigen binding fragment thereof disclosed in Table 16 are contemplated herein in methods of enhancing T cell activity (e.g., increasing cytokine production or increasing T cell proliferation).

In one specific embodiment, the anti-PD-1 monoclonal antibody or antigen binding fragment thereof is pembrolizumab. In another specific embodiment, the anti-PD-1 monoclonal antibody or antigen binding fragment thereof is nivolumab. In another specific embodiment, the anti-PD-1 monoclonal antibody or antigen binding fragment thereof is cemiplimab.

In certain embodiments of various methods of increasing cytokine production of T cells, the cytokine is selected from the group consisting of IL-1, IL-2, IL-6, IL-12, IL-17, IL-22, IL-23, GM-CSF, IFN-γ, and TNF-α. In one embodiment, the cytokine is IL-1. In another embodiment, the cytokine is IL-2. In yet another embodiment, the cytokine is IL-6. In still another embodiment, the cytokine is IL-12. In one embodiment, the cytokine is IL-17. In another embodiment, the cytokine is IL-22. In yet another embodiment, the cytokine is IL-23. In still another embodiment, the cytokine is GM-CSF. In one embodiment, the cytokine is IFN-γ. In another embodiment, the cytokine is TNF-α. In some embodiments, the cytokine is one, two, three, four, five, six, seven, eight, nine, or ten cytokines selected from the group consisting of IL-1, IL-2, IL-6, IL-12, IL-17, IL-22, IL-23, GM-CSF, IFN-γ, and TNF-α.

### 8. Dosing and Administration

Further provided herein are dosing regimens and routes of administration for treating cancer (e.g., colorectal cancer) or an infectious disease (e.g., a viral infection) using a combination of an anti-human PD-1 monoclonal antibody or antigen binding fragment thereof, an anti-human CTLA4 monoclonal antibody or antigen binding fragment thereof, and an anti-human TIGIT monoclonal antibody or antigen binding fragment thereof.

The anti-PD-1 monoclonal antibody or antigen binding fragment thereof, the anti-CTLA4 monoclonal antibody or antigen binding fragment thereof, and anti-TIGIT monoclonal antibody or antigen binding fragment thereof disclosed herein may be administered by continuous infusion, or by doses administered, e.g., daily, 1-7 times per week, weekly, bi-weekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. A total dose for a treatment interval is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more. Doses may also be provided to achieve a pre-determined target concentration of the antibody (eg., anti-PD-1 antibody) or antigen binding fragment thereof in the subject's serum, such as 0.1, 0.3, 1, 3, 10, 30, 100, 300 μg/mL or more.

In some embodiments, the anti-PD-1 monoclonal antibody or antigen binding fragment thereof is administered subcutaneously or intravenously, on a weekly, biweekly, triweekly, every 3 weeks, every 4 weeks, every 5 weeks, every 6 weeks, monthly, bimonthly, or quarterly basis at 10, 20, 50, 80, 100, 200, 300, 400, 500, 1000 or 2500 mg/subject. In some specific methods, the dose of the anti-PD-1 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-PD-1 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-PD-1 monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

In some embodiments, the anti-CTLA4 monoclonal antibody or antigen binding fragment thereof is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 500, 1000 or 2500 mg/subject. In some specific methods, the dose of the anti-CTLA4 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-CTLA4 monoclonal antibody or antigen binding fragment thereof is from about 1 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-CTLA4 monoclonal antibody or antigen binding fragment thereof is 1 mg, 5 mg, 10 mg, 15 mg, 20 mg, 30 mg, 40 mg, 45 mg, 50 mg, 75 mg, 100 mg, 125 mg, 150 mg, 175 mg, 200 mg, 225 mg, 240 mg, 250 mg, 300 mg, 400 mg, or 500 mg.

In some embodiments, the anti-TIGIT monoclonal antibody or antigen binding fragment thereof is administered subcutaneously or intravenously, on a weekly, biweekly, triweekly, every 4 weeks, every 5 weeks, every 6 weeks, monthly, bimonthly, or quarterly basis at 10, 20, 50, 80, 100, 200, 500, 1000 or 2500 mg/subject. In some specific methods, the dose of the anti-TIGIT monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-TIGIT monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-TIGIT monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

In a specific embodiment, the anti-human PD-1 monoclonal antibody or antigen binding fragment thereof is pembrolizumab. In another specific embodiment, the anti-human PD-1 monoclonal antibody or antigen binding fragment thereof is nivolumab. In another specific embodiment, the anti-human PD-1 monoclonal antibody or antigen binding fragment thereof is cemiplimab.

Thus, in certain embodiments, the human patient is administered:
(a) 200 mg of pembrolizumab;
(b) an anti-human CTLA4 monoclonal antibody or antigen binding fragment thereof; and
(c) an anti-human TIGIT monoclonal antibody or antigen binding fragment thereof; wherein pembrolizumab is administered once every three weeks.

In other embodiments, the human patient is administered:
(a) 2 mg/kg of pembrolizumab;
(b) an anti-human CTLA4 monoclonal antibody or antigen binding fragment thereof; and
(c) an anti-human TIGIT monoclonal antibody or antigen binding fragment thereof; wherein pembrolizumab is administered once every three weeks.

In some embodiments, the human patient is administered:
(a) 400 mg of pembrolizumab;
(b) an anti-human CTLA4 monoclonal antibody or antigen binding fragment thereof; and
(c) an anti-human TIGIT monoclonal antibody or antigen binding fragment thereof; wherein pembrolizumab is administered once every six weeks.

In some embodiments, the human patient is administered:
(a) 240 mg of nivolumab;
(b) an anti-human CTLA4 monoclonal antibody or antigen binding fragment thereof; and
(c) an anti-human TIGIT monoclonal antibody or antigen binding fragment thereof; wherein nivolumab is administered once every two weeks.

In other embodiments, the human patient is administered:
(a) 3 mg/kg of nivolurnab;
(b) an anti-human CTLA4 monoclonal antibody or antigen binding fragment thereof; and
(c) an anti-human TIGIT monoclonal antibody or antigen binding fragment thereof; wherein nivolumab is administered once every two weeks.

In some embodiments, the human patient is administered:
(a) 350 mg of cemiplimab;
(b) an anti-human CTLA4 monoclonal antibody or antigen binding fragment thereof; and
(c) an anti-human TIGIT monoclonal antibody or antigen binding fragment thereof; wherein pembrolizumab is administered once every three weeks.

In certain embodiments, the human patient is administered:
(a) an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein the anti-human PD-1 monoclonal antibody or antigen binding fragment thereof is administered once every three weeks.

In some embodiments, the human patient is administered:
(a) 200 mg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein the anti-human PD-1 monoclonal antibody or antigen binding fragment thereof is administered once every three weeks.

In other embodiments, the human patient is administered:
(a) 2 mg/kg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein the anti-human PD-1 monoclonal antibody or antigen binding fragment thereof is administered once every three weeks.

In some embodiments, the human patient is administered:
(a) 400 mg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:1, 2, and 3, respectively, and a V_{H} CDR1, a V_{H} CDR2, and a V_{H} CDR3 comprising amino acid sequences as set forth in SEQ ID NOS:6, 7, and 8, respectively;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein the anti-human PD-1 monoclonal antibody or antigen binding fragment thereof is administered once every six weeks.

In some embodiments, the human patient is administered:
(a) 200 mg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein the anti-human PD-1 monoclonal antibody or antigen binding fragment thereof is administered once every two weeks.

In other embodiments, the human patient is administered:
(a) about 2 mg/kg of an anti-human PD-1 monoclonal antibody or antigen binding fragment thereof that comprises a V_{L} CDR1, a V_{L} CDR2, and a V_{L} CDR3 comprising amino acid sequences as set forth in SEQ ID NOS:31, 32, and 33, respectively, and a V_{H} CDR1, a V_{H} CDR2, and a V_{H} CDR3 comprising amino acid sequences as set forth in SEQ ID NOS:36, 37, and 38, respectively;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein the anti-human PD-1 monoclonal antibody or antigen binding fragment thereof is administered once every two weeks.

In certain embodiments, the human patient is administered:
(a) about 200 mg of pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein pembrolizumab is administered once every three weeks.

In some embodiments, the human patient is administered:
(a) 200 mg of pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein pembrolizumab is administered once every three weeks.

In other embodiments, the human patient is administered:
(a) 2 mg/kg of pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein pembrolizumab is administered once every three weeks.

In some embodiments, the human patient is administered:
(a) 400 mg of pembrolizumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein pembrolizumab is administered once every six weeks.

In some embodiments, the human patient is administered:
(a) 240 mg of nivolumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein nivolumab is administered once every two weeks.

In other embodiments, the human patient is administered:
(a) 3 mg/kg of nivolumab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein nivolumab is administered once every two weeks.

In some embodiments, the human patient is administered:
(a) 350 mg of cemiplimab;
(b) an anti-human CTLA4 monoclonal antibody or antigen binding fragment thereof that comprises a V_{L} CDR1, a V_{L} CDR2, and a V_{L} CDR3 comprising amino acid sequences as set forth in SEQ ID NOS:11, 12, and 13, respectively, and a V_{H} CDR1, a V_{H} CDR2, and a V_{H} CDR3 comprising amino acid sequences as set forth in SEQ ID NOS:16, 17, and 18, respectively; and
(c) an anti-human TIGIT monoclonal antibody or antigen binding fragment thereof that comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively; wherein pembrolizumab is administered once every three weeks.

In various embodiments of the methods, the anti-TIGIT antibody or antigen binding fragment thereof is administered in an effective amount. In various embodiments of the methods, the anti-CTLA4 antibody or antigen binding fragment thereof is administered in an effective amount.

In some embodiments, at least one of the therapeutic agents (e.g., the anti-PD-1 monoclonal antibody or binding fragment thereof, the anti-CTLA4 monoclonal antibody or binding fragment thereof, or the anti-TIGIT monoclonal antibody or binding fragment thereof) in the combination therapy is administered using the same dosage regimen (dose, frequency, and duration of treatment) that is typically employed when the agent is used as monotherapy for treating the same condition. In other embodiments, the patient receives a lower total amount of at least one of the therapeutic agents (e.g., the anti-PD-1 monoclonal antibody or binding fragment thereof, the anti-CTLA4 monoclonal antibody or binding fragment thereof, or the anti-TIGIT monoclonal antibody or binding fragment thereof) in the combination therapy than when the agent is used as monotherapy, e.g., smaller doses, less frequent doses, and/or shorter treatment duration.

A combination therapy disclosed herein may be used prior to or following surgery to remove a tumor and may be used prior to, during, or after radiation treatment.

In some embodiments, a combination therapy disclosed herein is administered to a patient who has not previously been treated with a biotherapeutic or chemotherapeutic agent, i.e., is treatment-naïve. In other embodiments, the combination therapy is administered to a patient who failed to achieve a sustained response after prior therapy with the biotherapeutic or chemotherapeutic agent, i.e., is treatment-experienced.

The therapeutic combination disclosed herein may be used in combination with one or more other active agents, including but not limited to, other anti-cancer agents that are used in the prevention, treatment, control, amelioration, or reduction of risk of a particular disease or condition (e.g., cancer). Such other active agents may be administered, by a route and in an amount commonly used therefor, contemporaneously or sequentially with a therapeutic combination of the present disclosure.

The one or more additional active agents may be co-administered with the anti-PD-1 monoclonal antibody or antigen binding fragment thereof, the anti-CTLA4 monoclonal antibody or antigen binding fragment thereof, or the anti-TIGIT monoclonal antibody or antigen binding fragment thereof. The additional active agent(s) can be administered in a single dosage form with one or more co-administered agent selected from the anti-PD-1 monoclonal antibody or antigen binding fragment thereof, the anti-CTLA4 monoclonal antibody or antigen binding fragment thereof, and the anti-TIGIT monoclonal antibody or antigen binding fragment thereof. The additional active agent(s) can also be administered in separate dosage form(s) from the dosage forms containing the anti-PD-1 monoclonal antibody or antigen binding fragment thereof, the anti-CTLA4 monoclonal antibody or antigen binding fragment thereof, and/or the anti-TIGIT monoclonal antibody or antigen binding fragment thereof.

The additional active agent can be, e.g., a chemotherapeutic, a biotherapeutic agent (including but not limited to antibodies or antigen binding fragments thereof that specifically bind to an antigen selected from the group consisting of: PD-L1, PD-L2, CTLA4, BTLA, TIM3, HVEM, GITR, CD27, ILT2, ILT3, ILT4, ILT5, SIRPα, NKG2A, NKG2C, NKG2E, TSLP, ILi0, VISTA, VEGF, EGFR, Her2/neu, VEGF receptors, other growth factor receptors, CD20, CD28, CD40, CD-40L, CD70, OX-40, 4-1BB, and ICOS).

The additional active agent can be selected from the group consisting of STING agonists, poly ADP ribose polymerase (PARP) inhibitors, mitogen-activated protein kinase (MEK) inhibitors, cyclin-dependent kinase (CDK) inhibitors, indoleamine 2,3-dioxygenase (IDO) inhibitors, tryptophan 2,3-dioxygenase (TDO) selective inhibitors, anti-viral compounds, antigens, adjuvants, anti-cancer agents, CTLA-4 pathway antagonists, lipids, liposomes, peptides, cytotoxic agents, chemotherapeutic agents, immunomodulatory cell lines, checkpoint inhibitors, vascular endothelial growth factor (VEGF) receptor inhibitors, topoisomerase II inhibitors, smoothen inhibitors, alkylating agents, anti-tumor antibiotics, anti-metabolites, retinoids, and immunomodulatory agents including but not limited to anti-cancer vaccines.

The additional active agent can be an anti-viral compound, including but not limited to, hepatitis B virus (HBV) inhibitors, hepatitis C virus (HCV) protease inhibitors, HCV polymerase inhibitors, HCV NS4A inhibitors, HCV NS5A inhibitors, HCV NS5b inhibitors, and human immunodeficiency virus (HIV) inhibitors.

The additional active agent can be a cytotoxic agent, including but not limited to, arsenic trioxide (sold under the tradename TRISENOX®), asparaginase (also known as L-asparaginase, and Erwinia L-asparaginase, sold under the tradenames ELSPAR® and KIDROLASE).

The additional active agent can be an chemotherapeutic agent, including but not limited to, abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-prolyl-1-Lproline-t-butylamide, cachectin, cemadotin, chlorambucil, cyclophosphamide, 3′,4′-didehydro-4′deoxy-8′-norvin-caleukoblastine, dinaciclib, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyurea andtaxanes, ifosfamide, liarozole, lonidamine, lomustine, MDV3100, mechlorethamine (nitrogen mustard), melphalan, mivobulin isethionate, rhizoxin, sertenef, streptozocin, mitomycin, methotrexate, taxanes, nilutamide, olaparib, onapristone, paclitaxel, prednimustine, procarbazine, RPR109881, selumetinib, stramustine phosphate, tamoxifen, tasonermin, taxol, tretinoin, vinblastine, vincristine, vindesine sulfate, and vinflunine, and pharmaceutically acceptable salts thereof.

The additional active agent can be a vascular endothelial growth factor (VEGF) receptor inhibitors, including but not limited to, bevacizumab (sold under the trademark AVASTIN by Genentech/Roche), axitinib (described in PCT International Patent Publication No. Wo01/002369), Brivanib Alaninate ((S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indol-6-yl)-2-[(4-pyridinylmethyl)amino]-3-pyridinecarboxamide. and described in PCT International Patent Application Publication No. WO02/068470), pasireotide (also known as SO 230, and described in PCT International Patent Publication No. WO02/010192), and sorafenib.

The additional active agent can be a topoisomerase II inhibitor, including but not limited to, etoposide and teniposide.

The additional active agent can be an alkylating agent, including but not limited to, 5-azacytidine, decitabine, temozolomide, dactinomycin (also known as actinomycin-D, melphalan, altretamine, carmustine, bendamustine, busulfan, carboplatin, lomustine, cisplatin, chlorambucil, cyclophosphamide, dacarbazine, altretamine, ifosfamide, procarbazine, mechlorethamine, streptozocin, thiotepa, and pharmaceutically acceptable salts thereof.

The additional active agent can be an anti-tumor antibiotic, including but not limited to, doxorubicin, bleomycin, daunorubicin daunorubicin liposomal (daunorubicin citrate liposome), mitoxantrone, epirubicin, idarubicin, and mitomycin C.

The additional active agent can be an anti-metabolite, including but not limited to, claribine, 5-fluorouracil, 6-thioguanine, pemetrexed (sold under the tradename ALIMTA®), cytarabine (also known as arabinosylcytosine (Ara-C)), cytarabine liposomal (also known as Liposomal Ara-C, sold under the tradename DEPOCYT™), decitabine (sold under the tradename DACOGEN©), hydroxyurea and fludarabine, floxuridine, cladribine (also known as 2-chlorodeoxyadenosine (2-CdA), methotrexate (also known as amethopterin, methotrexate sodium (MTX)), and pentostatin.

The additional active agent can be a retinoid, including but not limited to, alitretinoin, tretinoin, isotretinoin, and bexarotene.

### 9. Pharmaceutical Compositions

In yet another aspect, provided herein are pharmaceutical compositions comprising:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment
   thereof.

In certain embodiments, the pharmaceutical composition further comprises a pharmaceutically acceptable carrier.

The pharmaceutical compositions comprising an anti-human PD-1 monoclonal antibody or antigen binding fragment thereof, an anti-human CTLA4 monoclonal antibody or antigen binding fragment thereof, and an anti-human TIGIT monoclonal antibody or antigen binding fragment thereof can be prepared for storage by mixing the antibodies having the desired degree of purity with optionally physiologically acceptable carriers, excipients, or stabilizers (see, e.g., Remington, Remington's Pharmaceutical Sciences (18^{th} ed. 1980)) in the form of aqueous solutions or lyophilized or other dried forms.

The pharmaceutically acceptable carriers, excipients, or stabilizers are non-toxic to the cell or mammalian being exposed thereto at the dosage and concentrations employed. Often the pharmaceutically acceptable carrier is an aqueous pH buffered solution. Examples of pharmaceutically acceptable carriers include buffers, such as phosphate, citrate, acetate, and other organic acids; antioxidants, such as ascorbic acid; low molecular weight (e.g., fewer than about 10 amino acid residues) polypeptide; proteins, such as serum albumin, gelatin, or immunoglobulin; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The pharmaceutically acceptable carriers can also refer to a diluent, adjuvate (e.g., Freund's adjuvate (complete or incomplete)), excipient, or vehicle. Such carriers can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsions, tablets, pills, capsules, powders, sustained-release formulations, and the like.

### 10. Kits

In still another aspect, provided herein are kits comprising a therapeutic combination of the antibodies or antigen binding fragments thereof disclosed herein (e.g., an anti-PD-1 monoclonal antibody or antigen binding fragment thereof, an anti-CTLA4 monoclonal antibody or antigen binding fragment thereof, and an anti-TIGIT monoclonal antibody or antigen binding fragment thereof) or a pharmaceutical composition thereof, packaged into suitable packaging material. A kit optionally includes a label or packaging insert that include a description of the components or instructions for use in vitro, in vivo, or ex vivo, of the components therein.

In some embodiments, the kit comprises
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In certain embodiments, the kit further comprises instructions for administering to a human patient the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.

In one embodiment, the kit comprises: (a) a dosage of an anti-PD-1 monoclonal antibody or antigen binding fragment thereof, (b) a dosage of an anti-CTLA4 monoclonal antibody or antigen binding fragment thereof, (c) a dosage of an anti-TIGIT monoclonal antibody or antigen binding fragment thereof, and (d) instructions for administering to a human patient the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

In another embodiment, the kit comprises: (a) a dosage of about 200 mg of pembrolizumab; (b) a dosage of an anti-CTLA4 monoclonal antibody or antigen binding fragment thereof; (c) a dosage of an anti-TIGIT monoclonal antibody or antigen binding fragment thereof, and (d) instructions for administering to a human patient the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.

In yet another embodiment, the kit comprises: (a) a dosage of 200 mg or 2 mg/kg of pembrolizumab; (b) a dosage of an anti-CTLA4 monoclonal antibody or antigen binding fragment thereof, (c) a dosage of an anti-TIGIT monoclonal antibody or antigen binding fragment thereof; and (d) instructions for administering to a human patient the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.

In yet another embodiment, the kit comprises: (a) a dosage of 400 mg of pembrolizumab; (b) a dosage of an anti-CTLA4 monoclonal antibody or antigen binding fragment thereof; (c) a dosage of an anti-TIGIT monoclonal antibody or antigen binding fragment thereof, and (d) instructions for administering to a human patient the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.

In still another embodiment, the kit comprises: (a) a dosage of 240 mg of nivolumab; (b) a dosage of an anti-CTLA4 monoclonal antibody or antigen binding fragment thereof; (c) a dosage of an anti-TIGIT monoclonal antibody or antigen binding fragment thereof, and (d) instructions for administering to a human patient the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.

In yet another embodiment, the kit comprises: (a) a dosage of 350 mg of cemiplimab; (b) a dosage of an anti-CTLA4 monoclonal antibody or antigen binding fragment thereof; (c) a dosage of an anti-TIGIT monoclonal antibody or antigen binding fragment thereof, and (d) instructions for administering to a human patient the anti-human PD-1 monoclonal antibody or antigen binding fragment thereof, the anti-human CTLA4 monoclonal antibody or antigen binding fragment thereof, and the anti-human TIGIT monoclonal antibody or antigen binding fragment thereof.

In some embodiments, the kit comprises means for separately retaining the components, such as a container, divided bottle, or divided foil packet. A kit of this disclosure can be used for administration of different dosage forms, for example, oral and parenteral, for administration of the separate compositions at different dosage intervals, or for titration of the separate compositions against one another.

### 11. Uses of a Therapeutic Combination for Treating Cancer or Infection

In still another aspect, provided herein are uses of a therapeutic combination for treating cancer (e.g., colorectal cancer) or an infectious disease (e.g., a viral infection) in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment
   thereof.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., DLBCL or NHL), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In certain embodiments, the cancer is metastatic. In some embodiments, the cancer is relapsed. In other embodiments, the cancer is refractory. In yet other embodiments, the cancer is relapsed and refractory. For example, the cancer is a melanoma. In certain embodiments, the cancer is a treatment of PD-1 refractory melanoma.

In one embodiment, the cancer is osteosarcoma. In another embodiment, the cancer is rhabdomyosarcoma. In yet another embodiment, the cancer is neuroblastoma. In still another embodiment, the cancer is kidney cancer. In one embodiment, the cancer is leukemia. In another embodiment, the cancer is renal transitional cell cancer. In yet another embodiment, the cancer is bladder cancer. In still another embodiment, the cancer is Wilm's cancer. In one embodiment, the cancer is ovarian cancer. In another embodiment, the cancer is pancreatic cancer. In yet another embodiment, the cancer is breast cancer. In still another embodiment, the cancer is prostate cancer. In one embodiment, the cancer is bone cancer. In another embodiment, the cancer is lung cancer. In yet another embodiment, the cancer is non-small cell lung cancer. In still another embodiment, the cancer is gastric cancer. In one embodiment, the cancer is colorectal cancer. In another embodiment, the cancer is cervical cancer. In yet another embodiment, the cancer is synovial sarcoma. In still another embodiment, the cancer is head and neck cancer. In one embodiment, the cancer is squamous cell carcinoma. In another embodiment, the cancer is lymphoma. In one embodiment, the cancer is DLBCL. In another embodiment, the cancer is NHL. In yet another embodiment, the cancer is multiple myeloma. In still another embodiment, the cancer is renal cell cancer. In one embodiment, the cancer is retinoblastoma. In another embodiment, the cancer is hepatoblastoma. In yet another embodiment, the cancer is hepatocellular carcinoma. In still another embodiment, the cancer is melanoma. In one embodiment, the cancer is rhabdoid tumor of the kidney. In another embodiment, the cancer is Ewing's sarcoma. In yet another embodiment, the cancer is chondrosarcoma. In still another embodiment, the cancer is brain cancer. In one embodiment, the cancer is glioblastoma. In another embodiment, the cancer is meningioma. In yet another embodiment, the cancer is pituitary adenoma. In still another embodiment, the cancer is vestibular schwannoma. In one embodiment, the cancer is primitive neuroectodermal tumor. In another embodiment, the cancer is medulloblastoma. In yet another embodiment, the cancer is astrocytoma. In still another embodiment, the cancer is anaplastic astrocytoma. In one embodiment, the cancer is oligodendroglioma. In another embodiment, the cancer is ependymoma. In yet another embodiment, the cancer is choroid plexus papilloma. In still another embodiment, the cancer is polycythemia vera. In one embodiment, the cancer is thrombocythemia. In another embodiment, the cancer is idiopathic myelfibrosis. In yet another embodiment, the cancer is soft tissue sarcoma. In still another embodiment, the cancer is thyroid cancer. In one embodiment, the cancer is endometrial cancer. In another embodiment, the cancer is carcinoid cancer. In yet another embodiment, the cancer is refractory head and neck cancer. In still another embodiment, the cancer is relapsed/refractory NHL (rrNHL). In yet still another embodiment, the cancer is PD-1 refractory rrNHL. For example, the cancer is a melanoma. In certain embodiments, the cancer is a treatment of PD-1 refractory melanoma.

In one embodiment, provided herein is use of a therapeutic combination for treating colorectal cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(b) an anti-human TIGIT monoclonal antibody or antigen binding fragment thereof.

In some embodiments, provided herein is use of a therapeutic combination for treating gastric cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In other embodiments, provided herein is use of a therapeutic combination for treating head and neck cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In yet other embodiments, provided herein is use of a therapeutic combination for treating refractory head and neck cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In still other embodiments, provided herein is use of a therapeutic combination for treating lung cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.
In certain embodiments, provided herein is use of a therapeutic combination for treating non-small cell lung cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In some embodiments, provided herein is use of a therapeutic combination for treating breast cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In other embodiments, provided herein is use of a therapeutic combination for treating cervical cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In yet other embodiments, provided herein is use of a therapeutic combination for treating ovarian cancer in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof, and
(c) an anti-human TIGIT monoclonal antibody or antigen binding fragment thereof.

In still other embodiments, provided herein is use of a therapeutic combination for treating DLBCL in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In certain embodiments, provided herein is use of a therapeutic combination for treating NHL in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In some embodiments, provided herein is use of a therapeutic combination for treating rrNHL in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In other embodiments, provided herein is use of a therapeutic combination for treating naïve rrNHL in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In yet other embodiments, provided herein is use of a therapeutic combination for treating PD-1 refractory rrNHL in a human patient, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In still other embodiments, provided herein is use of a therapeutic combination for treating an infectious disease, wherein the therapeutic combination comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

In one embodiment, the infectious disease is viral infection. In another embodiment, the infectious disease is bacterial infection. In yet another embodiment, the infectious disease is parasitic infection. In still another embodiment, the infectious disease is fungal infection.

In certain embodiments, the viral infection is infection with a virus selected from the group consisting of human immunodeficiency virus (HIV), ebola virus, hepatitis virus A (HAV), hepatitis virus B (HBV), hepatitis virus C (HCV), herpes virus (e.g., VZV, HSV-I, HAV-6, HSV-II, CMV, Epstein Barr virus), adenovirus, influenza virus, flavivirus, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus, and arboviral encephalitis virus.

In some embodiments, the bacterial infection is infection with a bacteria selected from the group consisting of chlamydia, rickettsia, mycobacteria, staphylococci, streptococci, pneumonococci, meningococci, gonococci, klebsiella, proteus, serratia, pseudomonas, legionella, salmonella, bacilli, borriella, Corynebacterium diphtheriae, Vibrio cholerae, Clostridium tetan, Clostridium botulinum, Bacillus anthricis, Yersinia pestis, Mycobacterium leprae, and Mycobacterium lepromatosis.

In other embodiments, the fungal infection is infection with a fungus selected from the group consisting of Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

In yet other embodiments, the parasitic infection is infection with a parasite selected from the group consisting of Entamoeba histolytica, Balantidium coli, Naegleria fowleri, Acanthamoeba, Giardia lambia, Cryptosporidium, Pneumocystis carinii, Plasmodium vivax, Babesia microti, Trypanosoma brucei, Trypanosoma cruzi, Leishmania donovani, Toxoplasma gondii, Nippostrongylus brasiliensis.

In some embodiments, the anti-PD-1 monoclonal antibody or antigen binding fragment thereof is an anti-human PD-1 monoclonal antibody or antigen binding fragment thereof as disclosed in Table 16.

In some embodiments, the anti-CTLA4 monoclonal antibody or antigen binding fragment thereof is an anti-human CTLA4 monoclonal antibody or antigen binding fragment thereof as disclosed in Table 16.

In some embodiments, the anti-TIGIT monoclonal antibody or antigen binding fragment thereof is an anti-human TIGIT monoclonal antibody or antigen binding fragment thereof as disclosed in Table 16.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

In still another aspect is the use of any therapeutic combination disclosed herein that comprises:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof, for the manufacture of a medicament for the treatment of cancer.

A number of embodiments of the invention have been described. It will be understood that various modifications may be made without departing from the spirit and scope of the invention. It will be further understood that each embodiment may be combined with one or more other embodiments, to the extent that such a combination is consistent with the description of the embodiments. Accordingly, the following examples are intended to illustrate but not limit the scope of the invention described in the claims.

### EXAMPLES

The examples in this section are offered by way of illustration, and not by way of limitation.

### Example 1: A Combination of an Anti-Mouse PD-1 Antibody with an Anti-Mouse CTLA4 Antibody, and/or an Anti-Mouse TIGIT Antibody Exhibited Anti-Tumor Effect in a Syngeneic Mouse Carcinoma Model

An EMT6 syngeneic mouse carcinoma model was used for studying the anti-tumor efficacy of a combination of three anti-mouse monoclonal antibodies (anti-mouse PD-1, anti-mouse CTLA4, and anti-mouse TIGIT mAbs). Antitumor efficacy of anti-TIGIT 18G10-IgG2a as a single agent was seen in an MC38 colon carcinoma mouse model and also as a single agent and in combination with anti-PD-1 (muDX400) therapy in a murine subcutaneous CT26 colon carcinoma-tumor bearing model (data not shown).

### Antibodies

Surrogate anti-mouse PD-1, anti-mouse CTLA4, and anti-mouse TIGIT monoclonal antibodies and isotype control antibodies are described in Table 3.

TABLE 3
Antibodies and Formulation of Antibodies

| Antibody | | | | |
|---|---|---|---|---|
| Name | Target | Isotype | Source | Formulation |
| mIgG1 | Adenoviral | Mouse IgG1 | Merck | 20 mM sodium |
| D265A | hexon 25 | D265A/kappa | Research | acetate, 9% |
| isotype | | | Laboratories | sucrose, pH 5.5 |
| control | | | | |
| mIgG1 | Adenoviral | Mouse IgG1 | Merck | 10 mM sodium |
| isotype | hexon 25 | kappa | Research | phosphate, 75 |
| control | | | Laboratories | mM sodium |
| | | | | chloride, 3% |
| | | | | sucrose, pH 7.3 |
| mIgG2a | Unknown | Mouse | BioXCell | Phosphate- |
| isotype | specificity | IgG2a/kappa | Cat. No. | buffered saline, |
| control | | | BE0085 | pH 7.0 |
| muDX400 | Mouse | Mouse IgG1 | Merck | 20 mM sodium |
| | PD-1 | D265A/kappa | Research | acetate, 9% |
| | | | Laboratories | sucrose, pH 5.5 |
| 9D9 | Mouse | Mouse IgG2a | Merck | 20 mM sodium |
| | CTLA4 | | Research | acetate, 9% |
| | | | Laboratories | sucrose, pH 5.5 |
| C18G10 | Mouse | Rat/mouse | Merck | 20 mM sodium |
| | TIGIT | chimera, | Research | acetate, 9% |
| | | mouse IgG2a | Laboratories | sucrose, pH 5.5 |
| | | D265A/kappa | | |

Stocks of the antibodies were stored frozen at −80° C., and dosing solutions were prepared by diluting the antibodies in their respective formulation solutions.

### Animals Used for Mouse Carcinoma Model Studies

Murine subjects mice were purchased for these studies. Conventional animal chow and water were provided ad libitum. Animals were housed for a period of time prior to the start of the study.

### Tumor Cell Line(s)

EMT6 cancer cell line (ATCC, Manassas, Va.) was cultured as is well known in the art. EMT6 was established from a transplantable murine mammary carcinoma that arose in a BALB/cCRGL mouse after implantation of a hyperplastic mammary alveolar nodule. The resulting tumor line (named KHJJ) was propagated in BALB/cKa mice and adapted to tissue culture after the 25th animal passage, and the cell line was named EMT. EMT6 is a clonal isolate of EMT isolated in 1971 at Stanford University. The EMT6 cell line can be grown either in animals as a tumor or in tissue culture. See Rockwell S. In vivo-in vitro tumor systems: new models for studying the response of tumors to therapy. Lab. Anim. Sci. 27: 831-851, 1977 and Collingridge D R, Rockwell S. Pentoxifylline improves the oxygenation and radiation response of BA1112 rat rhabdomyosarcomas and EMT6 mouse mammary carcinomas. Int. J. Cancer 90: 256-264, 2000. PubMed: 11091349.

### Tumor Measurements and Body Weights

Tumor length and width were measured using electronic calipers, and tumor volume was determined using the formula Volume (mm³)=0.5×Length×Width², where length is the longer dimension. Animals were weighed and tumors were measured twice a week. To prevent bias, any outliers by weight or tumor volume were removed and the remaining mice were assigned into groups of 10 based on tumor volumes. If tumor volume reached or exceeded 2000 mm³ before the scheduled take down, animals were euthanized.

### Tumor Challenge Experiment(s)

### EMT6 Colon Carcinoma Challenge

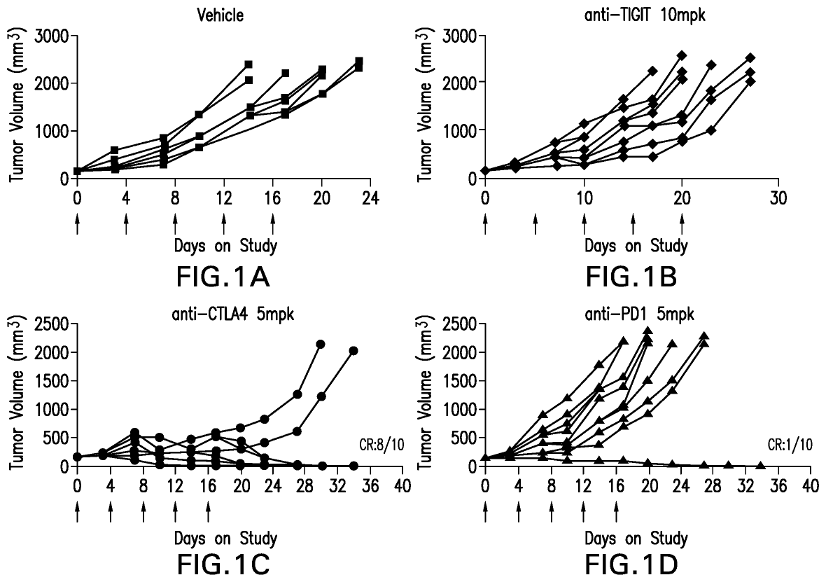

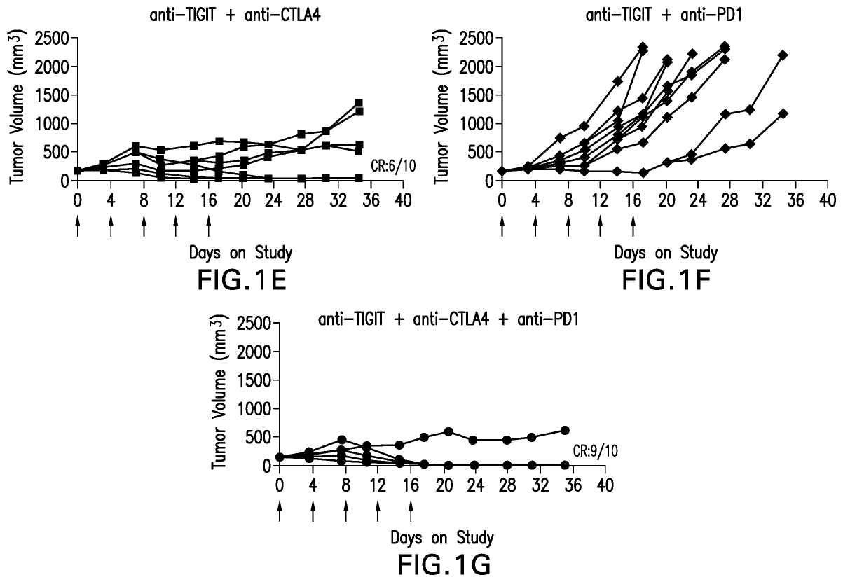

Briefly, Balb/c mice were subcutaneously implanted into the right hind flank with 0.5×EMT6 breast cancer cells. Tumors were grown until an average size of 150 mm³ at which point tumor-bearing animals were randomized to 7 groups of 10 animals each, and dosed intraperitoneally on days 0, 4, 8, 12, or 16 according to the following Table 4 and FIGS. 1A-G.

TABLE 4
Antibody and dosing information

| Group | Description | Dose (mg/kg) |
|---|---|---|
| A | Vehicle | |
| B | anti-TIGIT-IgG2a | 10 |
| C | anti-CTLA4_IgG2a | 5 |
| D | anti-PD-1-IgG1a | 5 |
| E | anti-TIGIT-IgG2a | 10 |
| | anti-CTLA4_IgG2a | 5 |
| F | anti-TIGIT-IgG2a | 10 |
| | anti-PD-1-IgG1a | 5 |
| G | anti-TIGIT-IgG2a | 10 |
| | anti-CTLA4_IgG2a | 5 |
| | anti-PD-1-IgG1a | 5 |

### Statistical Methods

Comparison of tumor volumes between treatments were made at each day of follow-up, and also collectively over all time points using area under the curve (AUC) as a summary measure for each tumor.

### Analysis of Tumor Volumes by Day

Follow-up of individual animals could be terminated early because of excessive tumor burden or other reasons. Depending on the reason and tumor size at the last measurement, the last observed tumor volume was treated as a lower bound on volume at all later days for that animal (right-censored data).

To compare two treatment groups on a given day, the Peto & Peto version of the Gehan-Breslow test for right-censored data was used (Klein and Moeschberger, Survival Analysis, 2^{nd} ed. Springer (2003)). In the absence of censoring, this reduces to the nonparametric Mann-Whitney (or Wilcoxon rank sum) test. Two-sided p-values were estimated from 20,000 random reassignments of animals between the two treatment groups being compared. To control the familywise error rate across all time points for a given pair of treatment groups, p-values were multiplicity adjusted by Holm's method. A p-value of less than 0.05 was used to define statistical significance.

For descriptive purposes, volumes for each day and treatment group were summarized by their median. To allow for censoring, a distribution function for each day and treatment group was estimated by the Kaplan-Meier method, with confidence band obtained using the beta product confidence procedure (Fay et al., Biostatistics, 14:723-736 (2013)). The median was estimated as the 50th percentile of the distribution function, with confidence interval obtained by inverting the confidence band. A 68% confidence level was used, to be comparable to the common “mean±SE” format for summarizing data, since the latter is approximately a 68% confidence interval for the mean.

When follow-up of an animal was terminated early, the reason was categorized and the animal's data were handled as follows: (1) tumor burden: right-censor at last measured value; (2) tumor ulceration; found dead, metastases found; found dead, unknown reason; protocol deviation; administrative; accident: right-censor at last measured value, provided this exceeded a threshold (1000 mm³); otherwise omit animal at later times; (3) weight loss/ill; acute treatment toxicity; found dead, with evidence of illness: omit animal at later times.

### Treatment Results

EMT6 tumor-bearing murine subjects were grouped into seven treatment groups on Day 0 when the mean volume of tumors reached approximately 150 mm³. The subjects were then treated with the previously described materials: (FIG. 1A) vehicle; (FIG. 1B) anti-TIGIT mIg2a (10 mpk); (FIG. 1C) anti-CTLA4 mIgG2a (5 mpk); (FIG. 1D) anti-mPD-1 mIgG1 (5 mpk); (FIG. 1E) anti-TIGIT mIg2a+anti-CTLA4 mIgG2a; (FIG. 1F) anti-TIGIT mIg2a+anti-mPD-1 mIgG1; and (FIG. 1G) anti-TIGIT mIg2a+anti-CTLA4 mIgG2a+anti-mPD-1 mIgG1. Complete regression (CR) of a tumor was defined as the absence of a measurable tumor. These results are shown in FIGS. 1 A-G.

It was observed that a tri-combination of anti-PD-1 mIgG1, anti-TIGIT mIgG2a, and anti-CTLA4 mIgG2a (FIG. 1G) resulted in reduced tumor volumes by Day 32 and Day 36. Most importantly, the tri-combination of antibodies blocking PD-1, TIGIT and CTLA4 reduced tumor volumes more than a single antibody treatment blocking PD-1 alone, a single antibody treatment blocking TIGIT alone, a single antibody treatment blocking CTLA4 alone, a combination treatment including an anti-PD-1 antibody and an anti-TIGIT antibody, and a combination treatment including an TIGIT antibody and an anti-CTLA4 antibody. See FIGS. 1A-G. In addition, the data showed that a combination treatment targeting TIGIT, PD-1 and CTLA4 is superior to a combination treatment targeting both TIGIT and PD-1 and is superior to a combination treatment targeting both CTLA4 and PD-1.

### Example 2. Anti-Tumor Effect of Anti-TIGIT mIgG2a+/−Anti-CTLA-4 mIgG2a+/−Anti-PD1 mIgG1 in the MBT2, Renca (200 mm³), EMT6 (150 mm³), MB49 Syngeneic Tumor Models

Further studies were performed to evaluate the anti-tumor effect of a combination treatment encompassing anti-TIGIT+/−anti-PD1/anti-CTLA-4 antibodies. MBT2, Renca (200 mm³), EMT6 (200 mm³), MB49 syngeneic tumor models were utilized in the studies.

### Study Design

### Animal Strains and Supplier/Vendors

Female mice of various strains were purchased from corresponding suppliers/vendors listed in Table 5. The animals were 7 to 8 weeks of age and weigh approximately 18 to 22 g at time of tumor cell inoculation. A total of 220 (80% spare) animals were needed for the each study (per strain vendor).

### Cell Culture

The cell culture condition for each line is listed in Table 5. For example, MBT2 mouse bladder cancer cell line was maintained as monolayer culture in DMEM supplemented with 10% heat inactivated fetal bovine serum (FBS) at 37° C. in an atmosphere with 5% C02 according to ATCC culture information. The tumor cells were sub-cultured 2-3 times per week depending on the growth rate and split ratio. The cells growing in an exponential growth phase were harvested and centrifuged at 335×g RCF in a refrigerated centrifuge and the medium aspirated. For cell inoculation, cell pellet were re-suspended in 10× volume serum-free DMEM, filtered through a 70 μm nylon mesh cell strainer and counted. The cell suspension was centrifuged again as above and re-suspended in serum-free DMEM to obtain 5×10⁶ cells per mL. The cell density was different depending on the number of cells for each inoculation (10× inoculation density, so each 0.1 mL delivered the amount of cells needed per inoculation of different cell line). Cells used for inoculation were passaged no more five times in culture after seeding from a frozen vial.

TABLE 5
Cell Culture, Mouse Strain/Vendor and Inoculation Information

| | | | | # Cells | Media (+ |
|---|---|---|---|---|---|
| Cell Line | Tissue | Strain | Vendor | implant | 10% FBS) |
| MBT-2 | Bladder | C3H/HeNTac | Taconic | 0.5 × 10\hat{ }6 | DMEM |
| EMT6 | Breast | BALB/cAnNT | Taconic | 0.5 × 10\hat{ }6 | DMEM |
| | | ac | | | |
| MB49 | Bladder | C57BL/6 | Jackson | 0.5 × 10\hat{ }6 | DMEM |
| RENCA | Renal | BALB/cAnNT | Taconic | 1 × 10\hat{ }6 | RPMI-1640 |
| | | ac | | | |

### Tumor Cell Inoculation and Randomization

Murine subjects were shaved in the lower right flank (near the dorsal thigh area) at least 24 hours prior to cell inoculation. Each animal was inoculated subcutaneously into the right lower flank with the single cell suspension of ≥95% viable tumor cells (the number cells required for each inoculation is 0.5×10⁶ MBT2 cells) in 0.1 mL of serum-free DMEM.

Mice were lightly anesthetized before implantation. Care was taken to ensure subcutaneous delivery of cells by lifting up the fold of skin with one hand and injection of cells with the other. During implantation, a new syringe and needle was used for every mouse inoculated, to minimize tumor ulceration. Any droplets on the skin were removed after injection to further minimize chance for tumor ulceration.

Animals were weighed and assigned to treatment groups using a randomization procedure. Animals were randomized into treatment groups when the mean tumor size reached approximately 100 mm³. The study was designed to last for approximately 21-28 days. Each efficacy group consisted of 10 mice. Mice were assigned to different treatment groups with equivalent mean tumor volume as shown in Table 6 under ‘Groups and Treatment’.

The acceptable range of EMT6 and Renca model tumor volume for enrolment into study was 120-220 mm³. Since tumor size can affect the effectiveness of any given treatment, animals were randomized into groups based upon their tumor sizes. Any tumors that completely or partially grew intradermally (ID) or intramuscularly (IM) were not be used for an efficacy study. Also, irregularly shaped (W or U-shaped) tumors were not used for an efficacy study.

### Group Designation and Dosing Schedule

All groups consisted of 10 animals per each group, and the test articles were administered according to the predetermined regimen listed in Table 6.

### Order and Timing of Dose Delivery

After dosing was completed, scientists remained with the mice for at least 1 hour. Any signs of lethargy, coldness to touch, slowed activity, or moribund status were noted along with any observations. Any moribund (not moving upon gentle prodding) mice were euthanized.

### Study Conduct and Animal Welfare

This protocol and/or procedures involving the care and use of animals in this study was reviewed and approved by the Institutional Animal Care and Use Committee (IACUC) prior to conduct. During the study, the care and use of animals was conducted in accordance with the regulations of the Association for Assessment and Accreditation of Laboratory Animal Care (AAALAC).

### Animal Housing

An acclimation period of approximately one week was allowed between animal receipt and tumor inoculation in order to accustom the animals to the laboratory environment. Animals were kept in a special pathogen-free environment in micro isolator cages (5 animals per cage). All cages, bedding, and water were sterilized before use. The targeted conditions for animal room environment and photoperiod will be as follows:
Temperature: 22° C.±3° C.
Humidity: 50%±20%
Light cycle: 12 hours light, 12 hours dark; (+/−2 hr)

### Food and Water

All animals had free access to a standard certified commercial laboratory diet. Maximum allowable concentrations of contaminants in the diet are controlled and routinely analyzed by the manufacturers. Autoclaved municipal tap water suitable for human consumption were available to the animals ad libitum.

TABLE 6
Efficacy groups and treatments

| | Group | Dose, | Dosing | | Group |
|---|---|---|---|---|---|
| Group | treatment | mg/kg | Schedule | ROA | Size |
| 1 | muIgG1 Iso + | 5 | Q5Dx5 | IP | 10 |
| | muIgG2a | 10 | Q5Dx5 | | |
| 2 | muIgG2a + | 10 | Q5Dx5 | IP | 10 |
| | mDX400 | 5 | Q5Dx5 | | |
| 3 | muIgG1 + | 5 | Q5Dx5 | IP | 10 |
| | TIGIT | 10 | Q5Dx5 | | |
| 4 | muIgG1 | 5 | Q5Dx5 | IP | 10 |
| | anti-CTLA4 | 1 | Q5Dx5 | | |
| 5 | muIgG1 | 5 | Q5Dx5 | IP | 10 |
| | anti-CTLA4 | 1 | Q10Dx5 | | |
| 6 | mDX400 | 5 | Q5Dx5 | IP | 10 |
| | TIGIT | 10 | Q5Dx5 | | |
| 7 | mDX400 + | 5 | Q5Dx5 | IP | 10 |
| | anti-CTLA4 | 1 | Q5Dx5 | | |
| 8 | mDX400 + | 5 | Q5Dx5 | IP | 10 |
| | anti-CTLA4 | 1 | Q10Dx5 | | |
| 9 | mIgG1 + | 5 | Q5Dx5 | IP | 10 |
| | TIGIT + | 10 | Q5Dx5 | | |
| | Anti-CTLA4 | 1 | Q5Dx5 | | |
| 10 | mIgG1 + | 5 | Q5Dx5 | IP | 10 |
| | TIGIT + | 10 | Q5Dx5 | | |
| | Anti-CTLA4 | 1 | Q10Dx5 | | |
| 11 | mDX400 + | 5 | Q5Dx5 | IP | 10 |
| | TIGIT + | 10 | Q5Dx5 | | |
| | Anti-CTLA4 | 1 | Q5Dx5 | | |
| 12 | mDX400 + | 5 | Q5Dx5 | IP | 10 |
| | TIGIT + | 10 | Q5Dx5 | | |
| | Anti-CTLA4 | 1 | Q10Dx5 | | |

IP = intraperitoneal.
¹ Administration volume: 100 ul/mouse

### Animal Observation Procedures

The protocol and any amendment(s) or procedures involving the care and use of animals in this study were reviewed and approved by the Institutional Animal Care and Use Committee (IACUC) of concerned institutions prior to conduct. During the study, the care and use of animals were conducted in accordance with the regulations of the Association for Assessment and Accreditation of Laboratory Animal Care (AAALAC). After inoculation, the animals were checked daily for morbidity and mortality. At the time of routine monitoring, the animals were checked for any effects of tumor growth on normal behavior such as mobility, food and water consumption, body weight gain/loss, eye/hair matting and any other abnormal effect. Death and observed clinical signs were recorded. Animals that are observed to be in a continuing deteriorating condition or bearing a tumor exceeding 2,000 mm³ in size were euthanized prior to death or before reaching a comatose state.

### Body Weights and Tumor Volumes

Body weight were measured 2-times per week. Tumor size was measured 3-times per week in 2 dimensions using a caliper (with one decimal points recorded).

Tumor volumes, expressed in mm³, were calculated using the following formula:

V (mm³)=(a×b²)/2,

where a and b are long and short diameters of a tumor, respectively.

The tumor size was then used for calculations of both tumor growth delay (T−C) and tumor growth inhibition (T/C) values (where T is the treatment group and C is the control group). T−C is calculated with T as the median time (in days) required for the treatment group tumors to reach a predetermined size (e.g., 1000 mm³), and C as the median time (in days) for the control group tumors to reach the same size. The T/C value (in percent) is an indication of antitumor effectiveness; T and C are the mean volume of the treated and control groups, respectively, on a given day.

### Efficacy Study Endpoints.

Tumor caliper and body weight data were captured at least twice per week until Day 30 relative to the start of dosing (+/−4 days). If some animals exited early study due to tumor size or other reason, the study for other animals in that group were continued. If some groups exited early study due to tumor size (for example, the isotype treated group), the study for the other groups was continued.

### Treatment Results

It was observed that a tri-combination of anti-PD-1 mIgG1, anti-TIGIT mIgG2a, and anti-CTLA4 mIgG2a resulted in significant reduced tumor volumes compared to all the single agent treatments. See Table 7 and Table 8. In addition, the data showed that a combination treatment targeting TIGIT, PD-1 and CTLA4 was at least as effective as, and in many cases superior to, a combination treatment targeting both TIGIT and PD-1, combination treatment targeting both CTLA4 and PD-1 and combination treatment targeting both TIGIT and CTLA4. In the MBT-2 mouse model, the triple combination treatment targeting TIGIT, PD-1 and CTLA4 was observed to be effective in multiple comparison dates to the dual combination treatments. See FIGS. 9A-H, FIGS. 10A-H and Table 9 for statistical analysis.

TABLE 7
Combination study results

| | | | | | | | Anti-PD-1 + |
|---|---|---|---|---|---|---|---|
| | | | Anti-PD-1 + | | Anti-PD-1 + | Anti-TIGIT + | Anti-TIGIT + |
| | Anti-PD-1 | Anti-TIGIT | Anti-TIGIT | Anti-CTLA-4 | anti-CTLA-4 | anti-CTLA-4 | anti-CTLA-4 |
| Model | (5 mpk) | (10 mpk) | (5, 10 mpk) | 1 mpk Q5d | 1 mpk Q5d | 1 mpk Q5d | 1 mpk Q5d |
| EMT6 | TGI: 59% | TGI: 30% | TGI: 62% | TGI: 97% | R: 74% | TGI: 94% | R: 70% |
| 150 mm3 | CR: 3/10 | | CR: 3/10 | CR: 8/10 | CR: 10/10 | CR: 8/10 | CR: 10/10 |
| (day 19*) | | | | | | | |
| Renca | TGI: 78% | TGI: 90% | R: 56% | R: 56% | R: 74% | R: 35% | R: 7% |
| 200 mm3 | CR: 3/10 | CR: 3/10 | CR: 9/10 | CR: 9/10 | CR: 10/10 | CR: 9/10 | CR: 9/10 |
| (day 17*) | | PR: 4/10 | PR: 1/10 | PR: 1/10 | | PR: 1/10 | PR: 1/10 |
| MB49 | TGI: 65% | TGI: 48% | TGI: 97% | TGI: 93% | TGI: 100% | TGI: 99% | TGI: 99% |
| 100 mm3 | | | CR: 3/10 | CR: 6/10 | CR: 9/10 | CR: 8/10 | CR: 10/10 |
| (day 15*) | | | | | | | |
| **MBT2 | TGI: 71% | TGI: 33% | TGI: 72% | TGI: 50% | TGI: 94% | TGI: 38% | TGI: 70% |
| 150 mm3 | PR: 1/10 | | CR: 1/10 | CR: 2/10 | CR: 2/10 | | CR: 1/10 |
| (Day 11*) | | | PR: 1/10 | | | | PR: 3/10 |

*Days that TGI and R % are calculated based on
**Anti-CTLA-4 (MBT2** The dosing level reduced to 1 mpk and 0.5 mpk Q7d respectively).

TABLE 8
Combination study results

| | | | | | | | Anti-PD-1 + |
|---|---|---|---|---|---|---|---|
| | | | Anti-PD-1 + | | Anti-PD-1 + | Anti-TIGIT + | Anti-TIGIT + |
| | Anti-PD-1 | Anti-TIGIT | Anti-TIGIT | Anti-CTLA-4 | anti-CTLA-4 | anti-CTLA-4 | anti-CTLA-4 |
| Model | (5 mpk) | (10 mpk) | (5), 10 mpk) | 1 mpk Q10d | 1 mpk Q10d | 1 mpk Q10d | 1 mpk Q10d |
| EMT6 | TGI: 59% | TGI: 30% | TGI: 62% | TGI: 97% | R: 81% | TGI: 98% | R: 67% |
| 150 mm3 | CR: 3/10 | | CR: 3/10 | CR: 8/10 | CR: 9/10 | CR: 9/10 | CR: 9/10 |
| (day 19*) | | | | | PR: 1/10 | | |
| Renca | TGI: 78% | TGI: 90% | R: 56% | TGI: 97% | R: 81% | TGI: 98% | R: 67% |
| 200 mm3 | CR: 3/10 | CR: 3/10 | CR: 9/10 | CR: 8/10 | CR: 9/10 | CR: 9/10 | CR: 9/10 |
| (day 17*) | | PR: 4/10 | PR: 1/10 | | PR: 1/10 | | |
| MB49 | TGI: 65% | TGI: 48% | TGI: 97% | TGI: 98% | R: 2% | TGI: 95% | TGI: 99% |
| 100 mm3 | | | CR: 3/10 | CR: 6/10 | CR: 9/10 | CR: 5/10 | CR: 8/10 |
| (day 15*) | | | | | PR: 1/10 | PR: 1/10 | PR: 1/10 |
| **MBT2 | TGI: 71% | TGI: 33% | TGI: 72% | TGI: 37% | TGI: 89% | TGI: 55% | TGI: 82% |
| 150 mm3 | PR: 1/10 | | CR: 1/10 | | CR: 2/10 | CR: 1/10 | CR: 2/10 |
| (Day 11*) | | | PR: 1/10 | | PR: 1/10 | | PR: 1/10 |

*Days that TGI and R % are calculated based on
**Anti-CTLA-4 (MBT2** The dosing level reduced to 1 mpk and 0.5 mpk Q7d respectively.

TABLE 9
Statistical analysis
Anti-TIGIT MBT-2
P-values for comparisons of Tumor volume between treatments.
Double vs Triple

| | | | Day | Day | Day | Day | Day | Day | Day | Day | Day | Day | Day |
|---|---|---|---|---|---|---|---|---|---|---|---|---|---|
| Groups being compared | | | 4 | 7 | 11 | 14 | 18 | 21 | 25 | 28 | 32 | 35 | 39 |
| Un- | mDX400 + | mDX400 + anti- | 0.05350 | 0.00235 | 0.00160 | 0.01440 | 0.02765 | 0.16185 | 0.39055 | 0.81310 | 0.48410 | 0.43685 | 0.25960 |
| adjusted | anti-CTLA4 | TIGIT + anti- | | | | | | | | | | | |
| p- | 1 mpk | CTLA4 1 mpk | | | | | | | | | | | |
| value* | mDX400 + | mDX400 + anti- | 0.97355 | 0.14410 | 0.02835 | 0.10475 | 0.14260 | 0.48280 | 0.67010 | 0.97175 | 0.63325 | 0.53315 | 0.47660 |
| | anti-CTLA4 | TIGIT + anti- | | | | | | | | | | | |
| | 0.5 mpk | CTLA4 0.5 mpk | | | | | | | | | | | |
| | anti-TIGIT + | mDX400 + anti- | 0.35835 | 0.31535 | 0.01985 | 0.00730 | 0.00065 | 0.00075 | 0.00085 | 0.00105 | 0.00090 | 0.00185 | 0.00480 |
| | anti-CTLA4 | TIGIT + anti- | | | | | | | | | | | |
| | 1 mpk | CTLA4 1 mpk | | | | | | | | | | | |
| | anti-TIGIT + | mDX400 + anti- | 0.12330 | 0.07285 | 0.00415 | 0.00340 | 0.00150 | 0.00185 | 0.00170 | 0.00295 | 0.00330 | 0.00295 | 0.01270 |
| | anti-CTLA4 | TIGIT + anti- | | | | | | | | | | | |
| | 0.5 mpk | CTLA4 0.5 mpk | | | | | | | | | | | |
| | mDX400 + | mDX400 + anti- | 0.25200 | 0.48390 | 0.63135 | 0.85350 | 0.35000 | 0.12775 | 0.10990 | 0.10705 | 0.10610 | 0.18165 | 0.28120 |
| | anti-TIGIT | TIGIT + anti- | | | | | | | | | | | |
| | | CTLA4 1 mpk | | | | | | | | | | | |
| | mDX400 + | mDX400 + anti- | 0.57690 | 0.63345 | 0.79690 | 0.47490 | 0.05365 | 0.03450 | 0.02275 | 0.02535 | 0.06675 | 0.12625 | 0.42585 |
| | anti-TIGIT | TIGIT + anti- | | | | | | | | | | | |
| | | CTLA4 0.5 mpk | | | | | | | | | | | |

### Example 3: Clinical Studies of Anti-PD-1, Anti-TIGIT Antibody, and Anti-CTLA-4 Antibody in Subjects with Cancer

This study is a phase 1/2, rolling arm, multicenter, open-label, adaptive design//umbrella platform study to evaluate within this substudy protocol the efficacy of an anti-TIGIT investigational agent and an anti-CTLA4 investigational agent with or without pembrolizumab for the treatment of PD-1 refractory melanoma. Male and female participants with melanoma who are at least 18 years of age will be enrolled in this substudy protocol.

When administered as doublets with pembrolizumab, anti-TIGIT (31C6) antibody and anti-CTLA4 antibody (8D2H2L2 variant) have both demonstrated increased anti-tumor efficacy compared to pembrolizumab monotherapy in preliminary studies of NSCLC. This is significant because NSCLC, like melanoma, is characterized by high responsiveness to immunotherapy relative to other solid tumor types. In an expansion cohort of the 31C6 antibody, ORR per RECIST1.1 by Investigator was 30.8% (4/13) confirmed and 46.2% (6/13) subjects with PD-L1+(TPS>1) PD1 naïve NSCLC. By contrast, the ORR observed for PD11+2L NSCLC patients treated with pembrolizumab monotherapy on Keynote-010 was 18%. Reck et al. Journal of Clinical Oncology 37, no. 7 (Mar. 1, 2019) 537-546. In a Phase 1 study of anti-CTLA4 antibody in advanced solid tumors, efficacy was similarly observed in patients treated with anti-CTLA4 antibody plus pembrolizumab in PD1 naïve 1L NSCLC (n=134). Efficacy was observed all doses and intervals tested, with confirmed ORR per RECIST 1.1 by central review was 39% at 25 mg Q3W, 33% at 25 mg Q6W, 22% at 75 mg Q6W, and 25% at 75 mg Q3W. Given the previously described murine data and the promising activity observed with immunotherapy doublet regimens in the IO responsive indication of NSCLC, it is believed that a triplet immunotherapy could provide additional clinical benefit over doublet therapy.

Preliminary efficacy from this substudy protocol will be evaluated by using ORR per RECIST 1.1, as determined by BICR. Throughout this protocol, the term RECIST 1.1 refers to the modification of RECIST 1.1 to include a maximum of target lesions and a maximum of 5 target lesions per organ. In this substudy protocol, an investigational treatment arm refers to a unique investigational agent or a combination of investigational agents with or without pembrolizumab. Investigational agents will only be added to this substudy protocol after an initial evaluation of safety and tolerability when administered alone and in combination with pembrolizumab has been completed. This substudy protocol will include participants with PD-1 refractory melanoma. PD-1 refractory melanoma must have progressed on treatment with an anti-PD-1/L1 mAb administered either as monotherapy, or in combination with other checkpoint inhibitors or other therapies. Participants must have received at least 2 doses of an approved anti-PD-1/L1 mAb and have demonstrated disease progression after PD-1/L1 as defined by RECIST 1.1. Throughout this protocol, the term RECIST 1.1 refers to the modification of RECIST 1.1 to include a maximum of 10 target lesions and a maximum of 5 target lesions per organ. Additional criteria are described herein.

The EORTC QLQ-C30 is the most widely used cancer-specific HRQoL instrument, which contains 30 items and measures 5 functional dimensions (physical, role, emotional, cognitive, and social), 3 symptom items (fatigue, nausea/vomiting, and pain), 6 single items (dyspnea, sleep disturbance, appetite loss, constipation, diarrhea, and financial impact), and a global health status/QoL scale (items 29 and 30). The EORTC QLQ-C30 is a psychometrically and clinically validated instrument appropriate for assessing QoL in oncology studies.

The EQ-5D-5L questionnaire is a standardized instrument for use as a measure of health outcome. The EQ-5D-5L will provide data for use in economic models and analyses including developing health utilities or quality-adjusted life years. The 5 health state dimensions in this instrument include the following: mobility, self-care, usual activities, pain/discomfort, and anxiety/depression. Each dimension is rated on a 5-point scale from 1 (no problem) to 5 (unable to/extreme problems). The EQ-5D-5L also includes a graded (0 to 100) vertical visual analog scale on which the participant rates their general state of health at the time of the assessment.

An exploratory objective of this substudy protocol is to characterize the PK profile of each investigational agent with or without pembrolizumab. The PK profile will be done for each agent separately. The serum concentrations of each investigational agent will be used to determine PK parameters (e.g., Cmax, AUC) for different investigational agents with or without pembrolizumab. Furthermore, the results of these analyses may be used in conjunction with safety and ADA endpoints to facilitate dosing strategies for the treatment combination.

Formation of ADAs can potentially confound drug exposures at therapeutic doses, and prime for subsequent infusion-related toxicity. Antidrug antibody response at the beginning of some selected cycles of treatment will be determined to potentially understand drug metabolism, exposure, and safety. Antidrug antibody response to different investigational antibody agents and pembrolizumab will evaluated in validated immunogenicity assays.

To identify novel biomarkers, biospecimens (i.e., blood components, tumor material) will be collected to support analyses of cellular components (e.g., protein, DNA, RNA, metabolites) and other circulating molecules. Investigations may include but are not limited to: germline (blood) genetic analyses (e.g., SNP analyses, whole exome sequencing, whole genome sequencing); genetic (DNA) analyses from any tumors; tumor and blood RNA analyses; proteomics and IHC using blood or tumor; and blood derived biomarkers.

Subjects are allocated into the following treatment arms:

### Investigational Treatment Arm A

Subjects are to be administered pembrolizumab (200 mg Q3W IV), anti-CTLA4 antibody 8D2H2L2 VARIANT 1 (light chain of SEQ ID NO: 53; heavy chain of SEQ ID NO: 52; 25 mg Q6W IV), and anti-TIGIT antibody (light chain of SEQ ID NO: 25; heavy chain of SEQ ID NO: 30; 200 mg Q3W IV). Treatment period is approximately 2 years. When more than 1 agent is to be given on the same day, the order of administration is pembrolizumab followed by anti-TIGIT antibody and then anti-CTLA4 antibody. Each of pembrolizumab, the anti-TIGIT antibody and the anti-CTLA4 antibody were by intravenous route of administration.

### Investigational Treatment Arm B

Subjects will receive pembrolizumab (at a fixed dose of 200 mg Q3W) in combination with anti-TIGIT antibody (at a fixed dose of 200 mg Q3W). Each of pembrolizumab, and the anti-TIGIT antibody were by intravenous route of administration.

### Investigational Treatment Arm C

Participants will receive 2 administrations of pembrolizumab (at a fixed dose of 200 mg Q3W) C1D1 and C2D1 in combination with anti-TIGIT antibody (at a fixed dose of 200 mg Q3W) C1D1 and C2D1 followed by surgical resection of the tumor. After surgical resection of the tumor, participants will receive 8 cycles of adjuvant therapy with pembrolizumab monotherapy (at a fixed dose of 400 mg Q6W) on Day 1 of every cycle (total treatment duration including neoadjuvant and adjuvant therapy of approximately 1 year). Each of pembrolizumab, and the anti-TIGIT antibody were by intravenous route of administration.

### Timing of Dose Administration

Study intervention will be administered on Day 1 of every treatment cycle. Pembrolizumab will be administered as a 30-minute IV infusion on Day 1 of every treatment cycle if given Q3W or on Day 1 of every other treatment cycle if given Q6W.

### Objectives and Endpoints

A listing of objectives and endpoints for the clinical trial substudy A, B, and C (described above) are shown in Table 10A, Table 10B, and Table 10C, respectively.

TABLE 10A
Objectives and endpoints for the clinical trial studyA

| Objectives | Endpoints |
|---|---|
| Primary | |
| To assess the safety and tolerability of | Adverse events. |
| investigational treatment combinations | Study intervention |
| based on the proportion of participants | discontinuations due to AEs. |
| with adverse events (AEs). | |
| To evaluate objective response rate | Objective Response: Complete |
| (ORR) as assessed by blinded | response (CR) or partial |
| independent central review (BICR) per | response (PR). |
| Response Evaluation Criteria in Solid | |
| Tumors 1.1 (RECIST 1.1). | |
| Secondary | |
| To evaluate the duration of response | DOR: For participants who |
| (DOR) as assessed by BICR per | demonstrate CR or PR, DOR |
| RECIST 1.1. | is defined as the time from the |
| | first documented evidence of |
| | CR or PR until disease |
| | progression or death due to any |
| | cause, whichever occurs first. |
| Tertiary/Exploratory | |
| To evaluate progression-free survival | PFS: the time from the date |
| (PFS) as assessed by BICR per | of randomization/allocation |
| RECIST 1.1. | to the date of the first |
| | documentation of disease |
| | progression or death from any |
| | cause, whichever occurs first. |
| To evaluate overall survival (OS). | OS: the time from the date of |
| | randomization/allocation to the |
| | date of death from any cause. |
| To evaluate ORR as assessed by the | Objective Response: CR or PR |
| investigator per RECIST 1.1. | |
| To evaluate objective response rate | Objective Response: CR or PR |
| (ORR) as assessed by investigator per | |
| RECIST 1.1 for Immune Based | |
| Therapeutics (iRECIST). | |
| To characterize the pharmacokinetic (PK) | Plasma concentration of agents |
| profile of each agent in the | combined with pembrolizumab/ |
| pembrolizumab-based/non- | other investigational agents. |
| pembrolizumab-based combinations. | |
| To evaluate the development of | Antidrug antibody (ADA) |
| circulating antidrug antibodies, of each | levels. |
| agent, following administration of | |
| pembrolizumab-based/non- | |
| pembrolizumab-based combinations. | |
| To identify molecular (genomic, | Molecular (genomic, |
| metabolic, and/or proteomic) biomarkers | metabolic, and/or proteomic) |
| that may be indicative of clinical | determinants of response or |
| response/resistance, safety, and/or the | resistance to treatments, using |
| mechanism of action of pembrolizumab | blood and/or tumor tissue. |
| and investigational agents. | |
| To assess the score change from baseline | Scores from the European |
| and the time to true deterioration (TTD) | Organization for Research |
| in patient-reported outcomes (PRO) | and Treatment of Cancer |
| scores in global health status/quality of | [EORTC] Quality of Life |
| life (QoL) and physical functioning. | Questionnaire-Core 30 |
| | [QLQ-C30]: |
| | Global health status/QoL scale |
| | (items 29 and 30) |
| | Physical functioning scale |
| | (items 1-5) |
| | TTD: the time to first onset |
| | of a 10 point or more decrease |
| | from baseline. TTD evaluated |
| | for EORTC QLQ-C30: |
| | Global health status/QoL scale |
| | (items 29 and 30) |
| | Physical functioning scale |
| | (items 1-5) |
| To evaluate health status as assessed by | Health utilities assessed using |
| the European Quality of Life 5-dimension | the EQ-5D-5L |
| 5-level (EQ-5D-5L) questionnaire to | |
| generate utility scores. | |
| To assess the effects of therapy on tumor | Profiles of longitudinal tumor |
| growth kinetics | response |

TABLE 10B
Objectives and endpoints for the clinical trial substudy B

| Objectives | Endpoints |
|---|---|
| To assess the safety and tolerability of | Adverse events. |
| investigational treatment combinations | Study intervention |
| based on the proportion of participants | discontinuations due to AEs. |
| with adverse events (AEs). | |
| To evaluate objective response rate | Objective Response: Complete |
| (ORR) as assessed by blinded | response (CR) or partial |
| independent central review (BICR) per | response (PR). |
| Response Evaluation Criteria in Solid | |
| Tumors 1.1 (RECIST 1.1). | |
| Secondary | |
| To evaluate the duration of response | DOR: For participants who |
| (DOR) as assessed by BICR per | demonstrate CR or PR, DOR |
| RECIST 1.1. | is defined as the time from the |
| | first documented evidence of |
| | CR or PR until disease |
| | progression or death due to |
| | any cause, whichever occurs first. |
| Tertiary/Exploratory | |
| To evaluate progression-free survival | PFS: the time from the date of |
| (PFS) as assessed by BICR per | randomization to the date of |
| RECIST 1.1. | the first documentation of |
| | disease progression or death |
| | from any cause, whichever |
| | occurs first. |
| To evaluate overall survival (OS). | OS: the time from the date of |
| | randomization to the date of |
| | death from any cause. |
| To evaluate ORR as assessed by the | Objective Response: CR or PR. |
| investigator per RECIST 1.1. | |
| To evaluate objective response rate | Objective Response: CR or PR. |
| (ORR) as assessed by investigator per | |
| RECIST 1.1 for Immune Based | |
| Therapeutics (iRECIST). | |
| To identify molecular (genomic, | Molecular (genomic, metabolic, |
| metabolic, and/or proteomic) biomarkers | and/or proteomic) determinants |
| that may be indicative of clinical | of response or resistance to |
| response/resistance, safety, and/or the | treatments, using blood and/or |
| mechanism of action of pembrolizumab | tumor tissue. |
| and investigational agents. | |
| To assess the effects of therapy on | Profiles of longitudinal tumor |
| tumor growth kinetics. | response. |

TABLE 10C
Objectives and endpoints for the clinical trial substudy C

| Objectives | Endpoints |
|---|---|
| Primary | |
| To assess the safety and tolerability of | Adverse events |
| investigational treatment combinations | Study intervention |
| based on the proportion of participants | discontinuations due to AEs |
| with adverse events (AEs). | |
| To evaluate pathological complete | pCR: Complete absence of |
| response (pCR) rate as assessed by | viable tumor in the treated |
| central review of the pathology results. | tumor bed. |
| Secondary | |
| To evaluate the near pathological | Near pCR: More than 0% but |
| complete response (near pCR) rate as | less than or equal to 10% of |
| assessed by central review of the | viable tumor cells in the |
| pathology results. | treated tumor bed. |
| To evaluate pathological partial response | pPR: More than 10% but less |
| (pPR) rate as assessed by central review | than or equal to 50% of viable |
| of the pathology results. | tumor cells in the treated |
| | tumor bed. |
| To evaluate recurrence-free survival | RFS: the time from the date of |
| (RFS) as assessed by the investigator. | surgery to (1) any recurrence |
| | (local, regional, or distant) or |
| | (2) death due to any cause |
| | (both cancer and noncancer |
| | causes of death). |
| Tertiary/Exploratory | |
| To evaluate overall survival (OS). | OS: the time from the date of |
| | randomization/allocation to the |
| | date of death from any cause. |
| To evaluate objective response rate | Objective Response: CR or PR |
| (ORR) as assessed by investigator per | |
| Response Evaluation Criteria in Solid | |
| Tumors 1.1 (RECIST 1.1). | |
| To characterize the pharmacokinetic | Serum concentration of agents |
| (PK) profile of select agents in the | combined with pembrolizumab/ |
| pembrolizumab-based/non- | other investigational agents. |
| pembrolizumab-based combinations. | |
| To evaluate the development of | Antidrug antibody (ADA) and/ |
| circulating antidrug antibodies, of select | or neutralizing antibody levels. |
| agents, following administration of | |
| pembrolizumab-based/non- | |
| pembrolizumab-based combinations. | |
| To identify molecular (genomic, | Molecular (genomic, metabolic, |
| metabolic, and/or proteomic) biomarkers | and/or proteomic) determinants |
| that may be indicative of clinical | of response or resistance to |
| response/resistance, safety, and/or the | treatments, using blood and/or |
| mechanism of action of pembrolizumab | tumor tissue. |
| and investigational agents. | |
| To evaluate the effects of neoadjuvant | Surgical outcomes (episodes |
| treatment on surgical outcomes. | of infection at the wound site |
| | requiring IV antibiotics and/ |
| | or wound drainage, number of |
| | episodes of seroma formation |
| | at the wound site requiring any |
| | intervention and volume of |
| | seroma drainage, number of |
| | episodes of bleeding from the |
| | wound requiring transfusion(s), |
| | incidence of lymphoedema). |
| To evaluate event-free survival (EFS) as | EFS: the time from the date of |
| assessed by the investigator. | randomization/allocation to the |
| | date of the first progression |
| | during neoadjuvant therapy |
| | (only in case of distant |
| | metastasis or local progression |
| | that precludes surgery with |
| | curative intent), recurrence |
| | (local, regional, or distant |
| | metastasis), or death from any |
| | cause, whichever occurs first. |

### Inclusion Criteria

A participant will be eligible for inclusion in the substudy if the participant:
1. Has histologically or cytologically confirmed melanoma.
2. Has unresectable Stage III or Stage IV melanoma, per AJCC 8th Edition Staging Criteria, not amenable to local therapy.
3. Has the presence of at least 1 measurable lesion by CT or MRI per RECIST 1.1 as confirmed by BICR.
a. Cutaneous lesions and other superficial lesions are not considered measurable lesions but may be considered as nontarget lesions.
b. If participants have only 1 measurable lesion per RECIST 1.1, the biopsy specimen should be obtained from the nontarget lesion or archival tissue. If biopsy specimen was obtained from a lone target lesion, a repeat screening CT must be obtained post biopsy and measurable disease confirmed by BICR.
c. Lesions that are in an area that has been previously irradiated should not be considered measurable unless there has been documented growth of the lesions since the completion of radiation.
   4. Has progressed on treatment with an anti-PD-1/L1 mAb administered either as monotherapy, or in combination with other checkpoint inhibitors or other therapies. PD-1 treatment progression is defined by meeting all of the following criteria:
a. Received at least 2 doses of an approved anti-PD-1/L1 mAb.
b. Demonstrated disease progression after PD-1/L1 as defined by RECIST 1.1. The initial evidence of disease progression is to be confirmed by a second assessment no less than 4 weeks from the date of the first documented progressive disease, in the absence of rapid clinical progression. This determination is made by the investigator. Once progressive disease is confirmed per iRECIST by the investigator, the initial date of progressive disease documentation will be considered the date of disease progression.
c. Progressive disease has been documented within 12 weeks from the last dose of anti-PD-1/L1 mAb.
d. Participants who receive anti-PD-1 therapy as adjuvant treatment following complete resection of Stage III or IV melanoma and have disease recurrence (unresectable loco-regional disease or distant metastases) while on active treatment or within 6 months of stopping anti-PD-1. For these participants, the following applies: 1) a second assessment to confirm disease progression beyond recurrence is not required; and 2) they must have received at least 2 prior doses of anti-PD-1/L1 mAb. This participant population may be limited to approximately 20% in each investigational treatment arm.
e. Prior therapy with CTLA-4 inhibitors will not be allowed.
   5. Has submitted pre-study imaging.
   Note: The site's study team must have reviewed prestudy images that are of diagnostic quality from at least 3 dates to determine that radiographic progression has occurred per RECIST 1.1/iRECIST following initiation of an anti-PD-1/L1 agent (see Section 8.2.1.1 for further details). The CIV must have received these scans and have confirmed that they are of acceptable diagnostic quality prior to treatment randomization/allocation in this study for a retrospective analysis of Inclusion Criterion 4: participants must be refractory to anti-PD-1/L1 agents. The CIV will not be confirming this eligibility criterion prior to treatment randomization/allocation.
   6. Has not received more than 3 lines of therapy for their advanced melanoma.
   7. Has provided a tumor biopsy.
a. Participants must submit tumor sample during Screening for confirmation of adequacy of tumor tissue at a central pathology laboratory. Participants who do not submit a tumor tissue sample will not be randomized/allocated.
b. Tumor sample should be freshly obtained (strongly preferred). In cases where newly obtained tissue is not possible to provide; an archival sample may be acceptable after discussion with the Sponsor. Only archival samples that have been obtained after progression on a PD-1/L1 agent will be accepted.
c. If a fresh tissue sample is submitted, it is preferred that the tumor biopsy is not obtained from a lone target lesion. If the biopsy specimen was obtained from a lone target lesion, a repeat screening CT must be obtained post-biopsy and measurable disease confirmed by BICR.

### Exclusion Criteria

The participant must be excluded from the substudy if the participant:

### Medical Conditions

1. Has a diagnosis of immunodeficiency or is receiving chronic systemic steroid therapy (in dosing exceeding 10 mg daily of prednisone equivalent) or any other form of immunosuppressive therapy within 7 days before the first dose of study intervention. Participants with asthma that require intermittent use of bronchodilators, inhaled steroids, or local steroid injections would not be excluded from the study.
2. Has a known additional malignancy that is progressing or requires active treatment within the past 2 years.
3. Has known active CNS metastases and/or carcinomatous meningitis. Has ocular or mucosal melanoma.
4. Has known hypersensitivity to active substances or any of their excipients including previous clinically significant hypersensitivity reaction to treatment with another mAb. For a list of excipients, refer to the respective IB.
5. Has an active autoimmune disease that has required systemic treatment in the past 2 years (i.e., with use of disease modifying agents, corticosteroids, or immunosuppressive drugs).
6. Has an active infection requiring systemic therapy.
7. Has known history of human immunodeficiency virus (HIV; HIV 1/2 antibodies). No testing of HIV is required unless mandated by local health authority.
8. Has known history of hepatitis B (defined as HBsAg reactive) or known hepatitis C virus (defined as HCV RNA [qualitative] is detected) infection.
9. Has a history of (noninfectious) pneumonitis that required steroids or current pneumonitis.
10. Has a history of active tuberculosis (TB; Bacillus tuberculosis).
11. A WOCBP who has a positive urine pregnancy test within 24 hours prior to randomization or treatment allocation.

### Prior/Concomitant Therapy

12. Has received prior systemic anticancer therapy including investigational agents within 4 weeks prior to randomization/allocation.
13. Has received prior radiotherapy within 2 weeks of first dose of study intervention.
14. Has had major surgery (<3 weeks prior to first dose of study intervention)
15. Has received a live vaccine within 30 days before the first dose of study intervention.

### Prior/Concurrent Clinical Study Experience

16. Is currently participating in or has participated in a study of an investigational agent or has used an investigational device within 4 weeks prior to the first dose of study intervention.

### Tumor Imaging During the Substudy

The first on-study imaging assessment should be performed at 9 weeks (63 days±7 days) from the date of randomization. Subsequent tumor imaging should be performed every 9 weeks (63 days±7 days) for the first year (until Week 52), after which the imaging interval increases to every 12 weeks (84 days+7 days) for −2 years (until Week 104); and Q24W thereafter or sooner if clinically indicated.

### Assessment at Screening and Prior to RECIST 1.1 Progression

Until radiographic disease progression based on RECIST 1.1, there is no distinct iRECIST assessment.

### Assessment and Decision at RECIST 1.1 Progression

For participants who show evidence of radiological progressive disease by RECIST 1.1 as determined by the investigator, the investigator will decide whether to continue a participant on study intervention until repeat imaging is obtained (using iRECIST for participant management). This decision by the investigator should be based on the participant's overall clinical condition.

Clinical stability is defined as the following:
Absence of symptoms and signs indicating clinically significant progression of disease
No decline in ECOG performance status
No requirements for intensified management, including increased analgesia, radiation, or other palliative care

Any participant deemed clinically unstable should be discontinued from study intervention at site-assessed first radiologic evidence of progressive disease and is not required to have repeat tumor imaging for confirmation of progressive disease by iRECIST.

If the investigator decides to continue treatment, the participant may continue to receive study intervention and the tumor assessment should be repeated 4 to 8 weeks later to confirm progressive disease by iRECIST, per investigator assessment. Images should continue to be sent in to the central imaging vendor for potential retrospective BICR.

Tumor flare may manifest as any factor causing radiographic progression per RECIST 1.1, including:
Increase in the sum of diameters of target lesion(s) identified at baseline to ≥20% and ≥5 mm from nadir
Note: The iRECIST publication uses the terminology “sum of measurements,” but “sum of diameters” will be used in this protocol, consistent with the original RECIST 1.1 terminology.
Unequivocal progression of nontarget lesion(s) identified at baseline
Development of new lesion(s)

iRECIST defines new response categories, including iUPD (unconfirmed progressive disease) and iCPD (confirmed progressive disease). For purposes of iRECIST assessment, the first visit showing progression according to RECIST 1.1 will be assigned a visit (overall) response of iUPD, regardless of which factors caused the progression.

At this visit, target and nontarget lesions identified at baseline by RECIST 1.1 will be assessed as usual.

New lesions will be classified as measurable or non-measurable using the same size thresholds and rules as for baseline lesion assessment in RECIST 1.1. From measurable new lesions, up to 5 lesions total (up to 2 per organ), may be selected as New Lesions—Target. The sum of diameters of these lesions will be calculated and kept distinct from the sum of diameters for target lesions at baseline. All other new lesions will be followed qualitatively as New Lesions— Nontarget.

### Assessment at the Confirmatory Imaging

On the confirmatory imaging, the participant will be classified as progression confirmed (with an overall response of iCPD), or as showing persistent unconfirmed progression (with an overall response of iUPD), or as showing disease stability or response (iSD/iPR/iCR).

### Confirmation of Progression

Progression is considered confirmed, and the overall response will be iCPD, if ANY of the following occurs:
Any of the factors that were the basis for the iUPD at the previous visit show worsening
   For target lesions, worsening is a further increase in the sum of diameters of ≥5 mm, compared to any prior iUPD time point
   For nontarget lesions, worsening is any significant growth in lesions overall, compared to a prior iUPD time point; this does not have to meet the “unequivocal” standard of RECIST 1.1
   For new lesions, worsening is any of these:
      An increase in the new lesion sum of diameters by ≥5 mm from a prior iUPD time point
      Visible growth of new nontarget lesions
      The appearance of additional new lesions
Any new factor appears that would have triggered progressive disease by RECIST 1.1
   Persistent iUPD

Progression is considered not confirmed, and the overall response remains iUPD, if:
None of the progression-confirming factors identified above occurs AND
The target lesion sum of diameters (initial target lesions) remains above the initial PD threshold (by RECIST 1.1)

Additional imaging for confirmation should be scheduled 4 to 8 weeks from the imaging on which iUPD is seen. This may correspond to the next visit in the original visit schedule. The assessment of the subsequent confirmation imaging proceeds in an identical manner, with possible outcomes of iCPD, iUPD, and iSD/iPR/iCR.

Resolution of iUPD

Progression is considered not confirmed, and the overall response becomes iSD/iPR/iCR, if:
None of the progression-confirming factors identified above occurs, AND
The target lesion sum of diameters (initial target lesions) is not above the initial progressive disease threshold.

The response is classified as iSD or iPR (depending on the sum of diameters of the target lesions), or iCR if all lesions resolve.

In this case, the initial iUPD is considered to be pseudo-progression, and the level of suspicion for progression is “reset.” This means that the next visit that shows radiographic progression, whenever it occurs, is again classified as iUPD by iRECIST, and the confirmation process is repeated before a response of iCPD can be assigned.

### Management Following the Confirmatory Imaging

If repeat imaging does not confirm progressive disease per iRECIST, as assessed by the investigator, and the participant continues to be clinically stable, study intervention may continue and follow the regular imaging schedule. If progressive disease is confirmed, participants will be discontinued from study intervention.

### Detection of Progression at Visits After Pseudo-progression Resolves

After resolution of pseudo-progression (i.e., achievement of iSD/iPR/iCR), iUPD is indicated by any of the following events:
Target lesions
   Sum of diameters reaches the progressive disease threshold (≥20% and ≥5 mm increase from nadir) either for the first time, or after resolution of previous pseudo-progression. The nadir is always the smallest sum of diameters seen during the entire substudy, either before or after an instance of pseudo-progression.
Nontarget lesions
   If nontarget lesions have never shown unequivocal progression, their doing so for the first-time results in iUPD.
   If nontarget lesions have shown previous unequivocal progression, and this progression has not resolved, iUPD results from any significant further growth of non-target lesions, taken as a whole.
New lesions
   New lesions appear for the first time
   Additional new lesions appear
   Previously identified new target lesions show an increase of ≥5 mm in the new lesion sum of diameters, from the nadir value of that sum
   Previously identified nontarget lesions show any significant growth

If any of the events above occur, the overall response for that visit is iUPD, and the iUPD evaluation process (see Assessment at the Confirmatory Imaging above) is repeated.

Progression must be confirmed before iCPD can occur. The decision process is identical to the iUPD confirmation process for the initial progressive disease, with 1 exception: If new lesions occurred at a prior instance of iUPD, and at the confirmatory imaging the burden of new lesions has increased from its smallest value (for new target lesions, the sum of diameters is ≥5 mm increased from its nadir), then iUPD cannot resolve to iSD or iPR. It will remain iUPD until either a decrease in the new lesion burden allows resolution to iSD or iPR, or until a confirmatory factor causes iCPD. Additional details about iRECIST are provided in the iRECIST publication [Seymour, L., et al 2017].

### AJCC 8^{th} Edition Staging Criteria and Overall Staging Table

The AJCC has designated staging by TNM classification to define melanoma. Staging tables (Table 11, Table 12, Table 13, and Table 14) adapted from AJCC 8th edition. Refer to AJCC guidelines for more information [Gershenwald, J. E., et al 2017]. ECOG performance status is listed in Table 15.

TABLE 11
Melanoma T Category Definition

| T Stage | T Stage Definition (thickness and ulceration) |
|---|---|
| TX | Primary tumor thickness cannot be assessed |
| | (ulceration status not applicable) |
| T0 | No evidence of primary tumor (ulceration status not |
| | applicable) |
| Tis | Melanoma in situ (ulceration status not applicable) |
| T1 | ≤1.0 mm (ulceration status unknown or unspecified) |
| T1a | <0.8 mm without ulceration |
| T1b | <0.8 mm with ulceration or |
| | 0.8-1.0 mm with or without ulceration |
| T2 | >1.0-2.0 mm (ulceration status unknown or |
| | unspecified) |
| T2a | >1.0-2.0 mm without ulceration |
| T2b | >1.0-2.0 mm with ulceration |
| T3 | >2.0-4.0 mm (ulceration status unknown or |
| | unspecified) |
| T3a | >2.0-4.0 mm without ulceration |
| T3b | >2.0-4.0 mm with ulceration |
| T4 | >4.0 mm (ulceration status unknown or unspecified) |
| T4a | >4.0 mm without ulceration |
| T4b | >4.0 mm with ulceration |

Abbreviations:
T = primary tumor.

TABLE 12
Melanoma N Category Definition

| | | Presence of |
|---|---|---|
| | | In-transit, Sat- |
| | | ellite, and/or |
| N | Number of Tumor-involved Regional Lymph | Microsatellite |
| Category | Nodes | Metastases |
| NX | Regional Nodes not assessed (exception: | No |
| | pathological N category not required for T1 | |
| | melanomas, use clinical N information) | |
| N0 | No regional metastases detected | No |
| N1 | One tumor-involved node or any number of in- | |
| | transit, satellite, and/or microsatellite | |
| | metastases with no tumor-involved nodes | |
| N1a | One clinically occult (detected by SLN biopsy) | No |
| N1b | One clinically detected | No |
| N1c | No regional lymph node disease | Yes |
| N2 | 2 or 3 tumor-involved nodes or any number of | |
| | in-transit, satellite, and/or micro-satellite | |
| | metastases with 1 tumor-involved node | |
| N2a | 2 or 3 clinically occult (detected by SLN | No |
| | biopsy) | |
| N2b | 2 or 3, at least one of which was clinically | No |
| | detected | |
| N2c | One clinically occult or clinically detected | Yes |
| N3 | Four or more tumor-involved nodes or any | |
| | number of in-transit, satellite, and/or | |
| | microsatellite metastases with 2 or more tumor- | |
| | involved nodes, or any number of matted nodes | |
| | without or with in-transit, satellite, and/or | |
| | microsatellite metastases. | |
| N3a | 4 or more clinically occult (detected by SLN | No |
| | biopsy) | |
| N3b | 4 or more, at least one of which was clinically | No |
| | detected, or the presence of any number of | |
| | matted nodes | |
| N3c | 2 or more clinically occult or clinically detected | Yes |
| | and/or presence of any number of matted nodes. | |

Abbreviations:
N = regional lymph node; SLN = sentinel lymph node.

TABLE 13
Melanoma M Category Definition

| | | Lactate |
|---|---|---|
| | | Dehydrogenase |
| M Category | Anatomic Site | Level |
| M0 | No evidence of distant metastasis | Not applicable |
| M1 | Evidence of distant metastasis | See below |
| M1a | Distant metastasis to skin, soft tissue | Not recorded or |
| | including muscle, and/or nonregional | unspecified |
| | lymph node | |
| M1a(0) | | Not elevated |
| M1a(1) | | Elevated |
| M1b | Distant metastasis to lung with or | Not recorded or |
| | without M1a sites of disease | unspecified |
| M1b(0) | | Not elevated |
| M1b(1) | | Elevated |
| M1c | Distant metastasis to noncentral nervous | Not recorded or |
| | system visceral sites with or without | unspecified |
| | M1a or M1b sites of disease | |
| M1c(0) | | Not elevated |
| M1c(1) | | Elevated |
| M1d | Distant metastasis to CNS with or | Not recorded or |
| | without M1a, M1b, or M1c sites of | unspecified |
| | disease | |
| M1d(0) | | Not elevated |
| M1d(1) | | Elevated |

Abbreviations:
CNS = central nervous system; M = distant metastasi

TABLE 14
AJCC Pathological (pTNM) Staging Groups
Staging (AJCC 8th edition)

| | | | |
|---|---|---|---|
| 0 | Tis | N0 | M0 |
| IA | T1a | N0 | M0 |
| IA | T1b | N0 | M0 |
| IB | T2a | N0 | M0 |
| IIA | T2b | N0 | M0 |
| IIA | T3a | N0 | M0 |
| IIB | T3b | N0 | M0 |
| IIB | T4a | N0 | M0 |
| IIC | T4b | N0 | M0 |
| IIIB | T0 | N1b, N1c | M0 |
| IIIC | T0 | N2b, N2c, N3b, N3c | M0 |
| IIIA | T1a/b − T2a | N1a or N2a | M0 |
| IIIB | T1a/b − t2a | N1b/c or N2b | M0 |
| IIIB | T2b/T3a | N1a − N2b | M0 |
| IIIC | T1a − T3a | N2c or N3a/b/c | M0 |
| IIIC | T3b/T4a | Any N ≥ N1 | M0 |
| IIIC | T4b | N1a − N2c | M0 |
| IIID | T4b | N3a/b/c | M0 |
| IV | Any T, Tis | Any N | M1 |

Abbreviations:
M0 = No evidence of distant metastases; N0 = No regional metastasis detected including no tumor-involved nodes and no in-transit, satellite, and/or microsatellite metastasis; pTNM = pathological tumor, nodes, and metastasis.

TABLE 15
ECOG Performance Status

| Grade | Description |
|---|---|
| 0 | Normal activity. Fully active, able to carry on all predisease |
| | performance without restriction. |
| 1 | Symptoms, but ambulatory. Restricted in physically strenuous |
| | activity, but ambulatory and able to carry out work of a light or |
| | sedentary nature (e.g., light housework, office work). |
| 2 | In bed <50% of the time. Ambulatory and capable of all self- |
| | care, but unable to carry out any work activities. Up and |
| | about >50% of waking hours. |
| 3 | In bed >50% of the time. Capable of only limited self-care, |
| | confined to bed or chair more than 50% of waking hours. |
| 4 | 100% bedridden. Completely disabled. Cannot carry on any |
| | self-care. Totally confined to bed or chair. |
| 5 | Dead. |

Abbreviations:
ECOG = Eastern Cooperative Oncology Group
Oken, M. M., Creech, R. H., Tormey, D. C, Horton, J., Davis, T. E., McFadden, E. T., Carbone, P. P.: Toxicity and Response Criteria of The Eastern Cooperative Oncology Group. Am J Clin Oncol 5: 649-655, 1982. The Eastern Cooperative Oncology Group, Robert Comis M.D., Group Chair.

### Quality-of-Life Assessments

Patient-reported outcomes will be assessed on Day 1 (prior to the first dose of study intervention) of every cycle, at the time of discontinuation (EOT visit), and the 30-day Safety Follow-up visit. If the EOT visit occurs 30 days from the last dose of study intervention a Safety Follow-up visit is not required and ePROs do not need to be repeated.

Every effort will be made to administer HRQoL surveys prior to administration of study intervention and before other assessments and procedures. Participants will complete the questionnaires in the following order: 1) EORTC QLQ-C30 and 2) EuroQoL EQ-5D-5L. If the participant does not complete the PROs at the designated time, the reason must be captured.

### Safety Assessments

Safety assessments include the collection of AEs and SAEs, monitoring of vital signs, physical examinations, performance of electrocardiograms (ECGs), a MUGA scan (using technetium-based tracer) or an ECHO at screening to assess LVEF), and pregnancy tests, among others.

### Physical Examinations

Complete physical examinations and brief directed physical examinations will be conducted by an investigator or medically qualified designee (consistent with local requirements) as per institutional standard at pre-determined timepoints. Clinical signs related to previous serious illnesses will be analyzed.

### Vital Signs

The investigator or qualified designee will measure vital signs at pre-determined timepoints. Vital signs include temperature, pulse, respiratory rate, and blood pressure. Weight will be monitored as per vital signs. Height will be measured at Screening only.

### Electrocardiograms

A standard 12-lead ECG will be performed during Screening using local standard procedures. Clinically important abnormal findings should be recorded as medical history. Additional ECGs may be performed as clinically necessary.

### Multiple-gated Acquisition Scan or Echocardiogram

All participants will undergo a MUGA scan (using technetium-based tracer) or an ECHO at screening to assess LVEF. MUGA or echocardiogram scans will be performed locally in accordance with the institution's standard practice.

### Clinical Safety Laboratory Assessments

### Urine Dipstick

All participants will undergo urine dipstick testing at Screening (within 3 days prior to the administration of the first dose of study intervention); testing will be performed locally. Participants with >1+proteinuria on urine dipstick at Screening will undergo 24-hour urine collection for quantitative assessment of proteinuria. Participants with urine protein ≥1 g/24-hr will not be eligible.

### Pregnancy Testing

All women who are being considered for participation in this substudy, and who are not surgically sterilized or postmenopausal, must be tested for pregnancy within 24 hours of the first dose of study intervention. If a urine test is positive or not evaluable, a serum test will be required. Participants must be excluded/discontinued from this substudy in the event of a positive test result. Repeated pregnancy testing (such as monthly testing) may be conducted if required by local regulation.

### Performance Assessments

ECOG Performance Scale: The investigator or qualified designee will assess ECOG status at the pre-determined timepoints.

### Adverse Events, Serious Adverse Events, and Other Reportable Safety Events

Adverse events, SAEs, and other reportable safety events will be reported by the participant (or, when appropriate, by a caregiver, surrogate, or the participant's legally authorized representative). The investigator, who is a qualified physician, will assess events that meet the definition of an AE or SAE as well as other reportable safety events with respect to seriousness, intensity/toxicity and causality.

### Safety Endpoints

The safety endpoints include AEs, SAEs, and study intervention discontinuation due to AEs. In addition, safety and tolerability will be assessed by clinical review of all relevant parameters including AEs, laboratory tests, and vital signs.

### Adverse Event (AE)

An AE is any untoward medical occurrence in a clinical study participant, temporally associated with the use of study intervention, whether or not considered related to the study intervention.
NOTE: An AE can therefore be any unfavorable and unintended sign (including an abnormal laboratory finding), symptom, or disease (new or exacerbated) temporally associated with the use of a study intervention.
   The following are included as AEs:
Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator.
Exacerbation of a chronic or intermittent pre-existing condition including either an increase in frequency and/or intensity of the condition.
New conditions detected or diagnosed after study intervention administration even though it may have been present before the start of the study.
Signs, symptoms, or the clinical sequelae of a suspected drug-drug interaction.
Signs, symptoms, or the clinical sequelae of a suspected overdose of either study intervention or a concomitant medication.
For all reports of overdose (whether accidental or intentional) with an associated AE, the AE term should reflect the clinical symptoms or abnormal test result. An overdose without any associated clinical symptoms or abnormal laboratory results is reported using the terminology “accidental or intentional overdose without adverse effect.”
   The following events do not meet the AE definition for the purpose of this study:
Medical or surgical procedure (e.g., endoscopy, appendectomy): the condition that leads to the procedure is the AE.
Situations in which an untoward medical occurrence did not occur (social and/or convenience admission to a hospital).
Anticipated day-to-day fluctuations of pre-existing disease(s) or condition(s) present or detected at the start of the study that do not worsen.
Surgery planned prior to informed consent to treat a pre-existing condition that has not worsened.

### Serious Adverse Event (SAE)

If an event is not an AE per the above, then it cannot be an SAE even if serious conditions are met. An SAE is defined as any untoward medical occurrence that, at any dose:
Results in death.
Is life-threatening. The term “life-threatening” in the definition of “serious” refers to an event in which the participant was at risk of death at the time of the event. It does not refer to an event, which hypothetically might have caused death, if it were more severe.
Requires inpatient hospitalization or prolongation of existing hospitalization. Hospitalization is defined as an inpatient admission, regardless of length of stay, even if the hospitalization is a precautionary measure for continued observation. (Note: Hospitalization for an elective procedure to treat a pre-existing condition that has not worsened is not an SAE. A pre-existing condition is a clinical condition that is diagnosed prior to the use of an MSD product and is documented in the participant's medical history.
Results in persistent or significant disability/incapacity. The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) that may interfere with or prevent everyday life functions but do not constitute a substantial disruption.
Is a congenital anomaly/birth defect. In offspring of participant taking the product regardless of time to diagnosis.
Other important medical events. Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent 1 of the other outcomes listed in the above definition. These events should usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

### Objective Response Rate (ORR)

The ORR is defined as the percentage of participants who achieve a confirmed CR or PR per RECIST 1.1 as assessed by BICR. Participants without follow-up scans will be considered non-responders. ORR as assessed by investigator per RECIST 1.1 and iRECIST are considered exploratory endpoints.

### Duration of Response (DOR)

For participants who demonstrate confirmed CR or PR per RECIST 1.1 as assessed by BICR, duration of response is defined as the time from the first documented evidence of CR or PR until disease progression or death due to any cause, whichever occurs first.

### Progression-free Survival (PFS)

Progression-free survival is defined as the time from date of randomization/allocation to the first documented progressive disease per RECIST 1.1 by BICR, or death due to any cause, whichever occurs first.

### Overall Survival (OS)

Overall survival is defined as the time from date of randomization/allocation to date of death from any cause.

The triplet combination of pembrolizumab 200 mg Q3W+MK-1308 25 mg Q6W+MK-7684 200 mg Q3W is currently being tested in a Phase 1/2 study in participants with unresectable or metastatic melanoma that is refractory to anti-PD-1/L1 therapy. As of Oct. 28, 2020, 3 participants had randomized to this triplet combination, and received their first dose.

### SEQUENCE LISTING

Table 16. below summarizes all sequences disclosed in the present specification.

TABLE 16
SEQ ID NOS and Corresponding Sequences

| SEQ | | |
|---|---|---|
| ID NO | Description | Sequence |
| 1 | Pembrolizumab, | RASKGVSTSGYSYLH |
| | VL-CDR1 | |
| 2 | Pembrolizumab, | LASYLES |
| | VL-CDR2 | |
| 3 | Pembrolizumab, | QHSRDLPLT |
| | VL-CDR3 | |
| 4 | Pembrolizumab, | EIVLTQSPATLSLSPGERATLSCRASKGVSTSGYSYLH |
| | VL | WYQQKPGQAPRLLIYLASYLESGVPARFSGSGSGTDF |
| | | TLTISSLEPEDFAVYYCQHSRDLPLTFGGGTKVEIK |
| 5 | Pembrolizumab, | EIVLTQSPATLSLSPGERATLSCRASKGVSTSGYSYLH |
| | light chain | WYQQKPGQAPRLLIYLASYLESGVPARFSGSGSGTDF |
| | | TLTISSLEPEDFAVYYCQHSRDLPLTFGGGTKVEIKRT |
| | | VAAPSVFIFPPSDEQLKSGTASVVCLLNNFYPREAKV |
| | | QWKVDNALQSGNSQESVTEQDSKDSTYSLSSTLTLS |
| | | KADYEKHKVYACEVTHQGLSSPVTKSFNRGEC |
| 6 | Pembrolizumab, | NYYMY |
| | VH-CDR1 | |
| 7 | Pembrolizumab, | GINPSNGGTNFNEKFKN |
| | VH-CDR2 | |
| 8 | Pembrolizumab, | RDYRFDMGFDY |
| | VH-CDR3 | |
| 9 | Pembrolizumab, | QVQLVQSGVEVKKPGASVKVSCKASGYTFTNYYMY |
| | VH | WVRQAPGQGLEWMGGINPSNGGTNFNEKFKNRVTL |
| | | TTDSSTTTAYMELKSLQFDDTAVYYCARRDYRFDMG |
| | | FDYWGQGTTVTVSS |
| 10 | Pembrolizumab, | QVQLVQSGVEVKKPGASVKVSCKASGYTFTNYYMY |
| | heavy chain | WVRQAPGQGLEWMGGINPSNGGTNFNEKFKNRVTL |
| | | TTDSSTTTAYMELKSLQFDDTAVYYCARRDYRFDMG |
| | | FDYWGQGTTVTVSSASTKGPSVFPLAPCSRSTSESTA |
| | | ALGCLVKDYFPEPVTVSWNSGALTSGVHTFPAVLQS |
| | | SGLYSLSSVVTVPSSSLGTKTYTCNVDHKPSNTKVDK |
| | | RVESKYGPPCPPCPAPEFLGGPSVFLFPPKPKDTLMIS |
| | | RTPEVTCVVVDVSQEDPEVQFNWYVDGVEVHNAKT |
| | | KPREEQFNSTYRVVSVLTVLHQDWLNGKEYKCKVS |
| | | NKGLPSSIEKTISKAKGQPREPQVYTLPPSQEEMTKNQ |
| | | VSLTCLVKGFYPSDIAVEWESNGQPENNYKTTPPVLD |
| | | SDGSFFLYSRLTVDKSRWQEGNVFSCSVMHEALHNH |
| | | YTQKSLSLSLGK |
| 11 | Anti-CTLA4 | GFTFSDNW |
| | VH-CDR1 | |
| 12 | Anti-CTLA4 | IRNKPYNYET |
| | VH-CDR2 | |
| 13 | Anti-CTLA4 | TAQFAY |
| | VH-CDR3 | |
| 14 | Anti-CTLA4 | ENIYGG |
| | VL-CDR1 | |
| 15 | Anti-CTLA4 | GAT |
| | VL-CDR2 | |
| 16 | Anti-CTLA4 | QNVLRSPFT |
| | VL-CDR3 | |
| 17 | Anti-CTLA4 | QNVLSRHPG |
| | VL-CDR3 | |
| 18 | Anti-CTLA4 | QNVLSSRPG |
| | VL-CDR3 | |
| 19 | Anti-CTLA4 | EVKLDETGGGLVQPGRPMKLSCVASGFTFSDNWMN |
| | VH 8D2/8D2 | WVRQSPEKGLEWLAQIRNKPYNYETYYSDSVKGRFT |
| | (RE) | ISRDDSKSSVYLQMNNLRGEDMGIYYCTAQFAYWG |
| | | QGTLVTVSA |
| 20 | Anti-CTLA4 | DIQMTQSPASLSASVGETVTITCGTSENIYGGLNWYQ |
| | VL 8D2/8D2 | RKQGKSPQLLIFGATNLADGMSSRFSGSGSGRQYSLK |
| | (RE) | ISSLHPDDVATYYCQNVLRSPFTFGSGTKLEI |
| 21 | Anti-TIGIT | RASEHIYSYLS |
| | antibody, | |
| | VL-CDR1 | |
| 22 | Anti-TIGIT | NAKTLAE |
| | antibody, | |
| | VL-CDR2 | |
| 23 | Anti-TIGIT | QHHFGSPLT |
| | antibody, | |
| | VL-CDR3 | |
| 24 | Anti-TIGIT | DIQMTQSPSSLSASVGDRVTITCRASEHIYSYLSWYQ |
| | antibody, VL | QKPGKVPKLLIYNAKTLAEGVPSRFSGSGSGTDFTLTI |
| | | SSLQPEDVATYYCQHHFGSPLTFGQGTRLEIK |
| 25 | Anti-TIGIT | DIQMTQSPSSLSASVGDRVTITCRASEHIYSYLSWYQ |
| | antibody, light | QKPGKVPKLLIYNAKTLAEGVPSRFSGSGSGTDFTLTI |
| | chain | SSLQPEDVATYYCQHHFGSPLTFGQGTRLEIKRTVAA |
| | | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| 26 | Anti-TIGIT | SYVMH |
| | antibody, VH- | |
| | CDR1 | |
| 27 | Anti-TIGIT | YIDPYNDGAKYAQKFQG |
| | antibody, VH- | |
| | CDR2 | |
| 28 | Anti-TIGIT | GGPYGWYFDV |
| | antibody, VH- | |
| | CDR3 | |
| 29 | Anti-TIGIT | EVQLVQSGAEVKKPGSSVKVSCKASGYTFSSYVMH |
| | antibody, VH | WVRQAPGQGLEWIGYIDPYNDGAKYAQKFQGRVTL |
| | | TSDKSTSTAYMELSSLRSEDTAVYYCARGGPYGWYF |
| | | DVWGQGTTVTVSS |
| 30 | Anti-TIGIT | EVQLVQSGAEVKKPGSSVKVSCKASGYTFSSYVMH |
| | antibody, heavy | WVRQAPGQGLEWIGYIDPYNDGAKYAQKFQGRVTL |
| | chain | TSDKSTSTAYMELSSLRSEDTAVYYCARGGPYGWYF |
| | | DVWGQGTTVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCDKTHTCPPCPAPELLGGPSVFLFPPKPKDTLM |
| | | ISRTPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSRDELTKN |
| | | QVSLTCLVKGFYPSDIAVEWESNGQPENNYKTTPPVL |
| | | DSDGSFFLYSKLTVDKSRWQQGNVFSCSVMHEALHN |
| | | HYTQKSLSLSPGK |
| 31 | Nivolumab, | RASQSVSSYLA |
| | VL-CDR1 | |
| 32 | Nivolumab, | DASNRAT |
| | VL-CDR2 | |
| 33 | Nivolumab, | QQSSNWPRT |
| | VL-CDR3 | |
| 34 | Nivolumab, VL | EIVLTQSPATLSLSPGERATLSCRASQSVSSYLAWYQ |
| | | QKPGQAPRLLIYDASNRATGIPARFSGSGSGTDFTLTI |
| | | SSLEPEDFAVYYCQQSSNWPRTFGQGTKVEIK |
| 35 | Nivolumab, | EIVLTQSPATLSLSPGERATLSCRASQSVSSYLAWYQ |
| | light chain | QKPGQAPRLLIYDASNRATGIPARFSGSGSGTDFTLTI |
| | | SSLEPEDFAVYYCQQSSNWPRTFGQGTKVEIKRTVAA |
| | | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |
| 36 | Nivolumab, | NSGMH |
| | VH-CDR1 | |
| 37 | Nivolumab, | VIWYDGSKRYYADSVKG |
| | VH-CDR2 | |
| 38 | Nivolumab, | NDDY |
| | VH-CDR3 | |
| 39 | Nivolumab, VH | QVQLVESGGGVVQPGRSLRLDCKASGITFSNSGMHW |
| | | VRQAPGKGLEWVAVIWYDGSKRYYADSVKGRFTISR |
| | | DNSKNTLFLQMNSLRAEDTAVYYCATNDDYWGQGT |
| | | LVTVSS |
| 40 | Nivolumab, | QVQLVESGGGVVQPGRSLRLDCKASGITFSNSGMHW |
| | heavy chain | VRQAPGKGLEWVAVIWYDGSKRYYADSVKGRFTISR |
| | | DNSKNTLFLQMNSLRAEDTAVYYCATNDDYWGQGT |
| | | LVTVSSASTKGPSVFPLAPCSRSTSESTAALGCLVKDY |
| | | FPEPVTVSWNSGALTSGVHTFPAVLQSSGLYSLSSVV |
| | | TVPSSSLGTKTYTCNVDHKPSNTKVDKRVESKYGPPC |
| | | PPCPAPEFLGGPSVFLFPPKPKDTLMISRTPEVTCVVV |
| | | DVSQEDPEVQFNWYVDGVEVHNAKTKPREEQFNST |
| | | YRVVSVLTVLHQDWLNGKEYKCKVSNKGLPSSIEKT |
| | | ISKAKGQPREPQVYTLPPSQEEMTKNQVSLTCLVKGF |
| | | YPSDIAVEWESNGQPENNYKTTPPVLDSDGSFFLYSR |
| | | LTVDKSRWQEGNVFSCSVMHEALHNHYTQKSLSLSL |
| | | GK |
| 41 | Anti-CTLA4 | EVQLVESGGGLVQPGGSMRLSCAASGFTFSDNWMN |
| | VH 8D2H1L1 | WVRQAPGKGLEWLAQIRNKPYNYETYYSDSVKGRF |
| | | TISRDDSKNSVYLQMNSLKTEDTGVYYCTAQFAYWG |
| | | QGTLVTVSS |
| 42 | Anti-CTLA4 | DIQMTQSPSSLSASVGDRVTITCRTSENIYGGLNWYQ |
| | VL 8D2H1L1 | RKQGKSPKLLIYGATNLASGMSSRFSGSGSGTDYTLK |
| | | IS SLHPDDV ATYYCQNVLRSPFTFGS GTKLEIK |
| 43 | Anti-CTLA4 | EVQLVESGGGLVQPGGSMRLSCAASGFTFSDNWMN |
| | VH 8D2H2L2 | WVRQAPGKGLEWLAQIRNKPYNYETYYSASVKGRF |
| | | TISRDDSKNSVYLQMNSLKTEDTGVYYCTAQFAYWG |
| | | QGTLVTVSS |
| 44 | Anti-CTLA4 | DIQMTQSPSSLSASVGDRVTITCRTSENIYGGLNWYQ |
| | VL 8D2H2L2 | RKPGKSPKLLIYGATNLASGVSSRFSGSGSGTDYTLTI |
| | and 8D2H2L2 | SSLQPEDVATYYCQNVLRSPFTFGSGTKLEIK |
| | VARIANT 1 | |
| 45 | Anti-CTLA4 | EVQLVESGGGLVQPGGSLRLSCAASGFTFSDNWMN |
| | VH 8D2H2L2 | WVRQAPGKGLEWLAQIRNKPYNYETYYSASVKGRF |
| | VARIANT 1 | TISRDDSKNSVYLQMNSLKTEDTGVYYCTAQFAYWG |
| | | QGTLVTVSS |
| 46 | Anti-CTLA4 | EVQLVESGGGLVQPGGSLRLSCAASGFTFSDNWMN |
| | VH 8D2H3L3 | WVRQAPGKGLEWVAQIRNKPYNYETEYAASVKGRF |
| | | TISRDDSKNSAYLQMNSLKTEDTAVYYCTAQFAYWG |
| | | QGTLVTVSS |
| 47 | Anti-CTLA4 | DIQMTQSPSSLSASVGDRVTITCRASENIYGGLNWYQ |
| | VL 8D2H3L3 | QKPGKAPKLLIYGATSLASGVPSRFSGSGSGTDYTLTI |
| | | SSLQPEDFATYYCQNVLRSPFTFGSGTKLEIK |
| 48 | Anti-CTLA4 | DIQMTQSPSSLSASVGDRVTITCRASENIYGGLNWYQ |
| | VH 8D2H2L15 | QKPGKAPKLLIYGATSLASGVPSRFSGSGSGTDYTLTI |
| | | SSLQPEDFATYYCQNVLRSPFTFGSGTKLEIK |
| 49 | Anti-CTLA4 | DIQMTQSPSSLSASVGDRVTITCRTSENIYGGLNWYQ |
| | VL 8D2H2L15 | RKPGKSPKLLIYGATNLASGVSSRFSGSGSGTDYTLTI |
| | | SSLQPEDVATYYCQNVLSRHPGFGSGTKLEIK |
| 50 | Anti-CTLA4 | EVQLVESGGGLVQPGGSMRLSCAASGFTFSDNWMN |
| | VH | WVRQAPGKGLEWLAQIRNKPYNYETYYSASVKGRF |
| | 8D2H2L17VL | TISRDDSKNSVYLQMNSLKTEDTGVYYCTAQFAYWG |
| | | QGTLVTVSS |
| 51 | Anti-CTLA4 | DIQMTQSPSSLSASVGDRVTITCRTSENIYGGLNWYQ |
| | VL 8D2H2L17 | RKPGKSPKLLIYGATNLASGVSSRFSGSGSGTDYTLTI |
| | | SSLQPEDVATYYCQNVLSSRPGFGSGTKLEIK |
| 52 | Anti-CTLA4 | EVQLVESGGGLVQPGGSLRLSCAASGFTFSDNWMN |
| | Full Heavy | WVRQAPGKGLEWLAQIRNKPYNYETYYSASVKGRF |
| | Chain | TISRDDSKNSVYLQMNSLKTEDTGVYYCTAQFAYWG |
| | 8D2H2L2 | QGTLVTVSSASTKGPSVFPLAPSSKSTSGGTAALGCL |
| | VARIANT 1 | VKDYFPEPVTVSWNSGALTSGVHTFPAVLQSSGLYSL |
| | | SSWTVPSSSLGTQTYICNVNHKPSNTKVDKKVEPKS |
| | | CDKTHTCPPCPAPELLGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LPAPIEKTISKAKGQPREPQVYTLPPSRDELTKNQVSL |
| | | TCLVKGFYPSDIAVEWESNGQPENNYKTTPPVLDSDG |
| | | SFFLYSKLTVDKSRWQQGNVFSCSVMHEALHNHYTQ |
| | | KSLSLSPGK |
| 53 | Anti-CTLA4 | DIQMTQSPSSLSASVGDRVTITCRTSENIYGGLNWYQ |
| | Full Light | RKPGKSPKLLIYGATNLASGVSSRFSGSGSGTDYTLTI |
| | Chain | SSLQPEDVATYYCQNVLRSPFTFGSGTKLEIKRTVAA |
| | 8D2H2L2 | PSVFIFPPSDEQLKSGTASVVCLLNNFYPREAKVQWK |
| | VARIANT 1 | VDNALQSGNSQESVTEQDSKDSTYSLSSTLTLSKADY |
| | | EKHKVYACEVTHQGLSSPVTKSFNRGEC |
| 54 | Anti-CTLA4 | RASQSVGSSYLA |
| | CDRL1 | |
| | (Ipilimumab) | |
| 55 | Anti-CTLA4 | GAFSRAT |
| | CDRL2 | |
| | (Ipilimumab) | |
| 56 | Anti-CTLA4 | GAFSRATQQYGSSPWT |
| | CDRL3 | |
| | (Ipilimumab) | |
| 57 | Anti-CTLA4 | SYTMH |
| | CDRH1 | |
| | (Ipilimumab) | |
| 58 | Anti-TIGIT | YIDPYNDGAKYNEKFKG |
| | antibody, VH- | |
| | CDR2 variant | |
| 59 | Anti-TIGIT | YIDPYNRGAKYNEKFG |
| | antibody, VH- | |
| | CDR2 variant | |
| 60 | Anti-TIGIT | YIDPYNLGAKYNEKFG |
| | antibody, VH- | |
| | CDR2 variant | |
| 61 | Anti-TIGIT | YIDPYNKGAKYNEKFG |
| | antibody, VH- | |
| | CDR2 variant | |
| 62 | Anti-TIGIT | YIDPYNFGAKYNEKFG |
| | antibody, VH- | |
| | CDR2 variant | |
| 63 | Anti-TIGIT | YIDPYNSGAKYNEKFG |
| | antibody, VH- | |
| | CDR2 variant | |
| 64 | Anti-TIGIT | YIDPYNYGAKYNEKFG |
| | antibody, VH- | |
| | CDR2 variant | |
| 65 | Anti-TIGIT | YIDPYNVGAKYNEKFG |
| | antibody, VH- | |
| | CDR2 variant | |
| 66 | Anti-TIGIT | YIDPYNDRAKYNEKFKG |
| | antibody, VH- | |
| | CDR2 variant | |
| 67 | Anti-TIGIT | YIDPYNDNAKYNEKFKG |
| | antibody, VH- | |
| | CDR2 variant | |
| 68 | Anti-TIGIT | YIDPYNDQAKYNEKFKG |
| | antibody, VH- | |
| | CDR2 variant | |
| 69 | Anti-TIGIT | YIDPYNDEAKYNEKFKG |
| | antibody, VH- | |
| | CDR2 variant | |
| 70 | Anti-TIGIT | YIDPYNDLAKYNEKFKG |
| | antibody, VH- | |
| | CDR2 variant | |
| 71 | Anti-TIGIT | YIDPYNDKAKYNEKFKG |
| | antibody, VH- | |
| | CDR2 variant | |
| 72 | Anti-TIGIT | YIDPYNDSAKYNEKFKG |
| | antibody, VH- | |
| | CDR2 variant | |
| 73 | Anti-TIGIT | YIDPYNDYAKYNEKFKG |
| | antibody, VH- | |
| | CDR2 variant | |
| 74 | Anti-TIGIT | YIDPYNDVAKYNEKFKG |
| | antibody, VH- | |
| | CDR2 variant | |
| 75 | Anti-TIGIT | YIDPYNDGAKYSQKFQG |
| | antibody, VH- | |
| | CDR2 variant | |
| 76 | Anti-TIGIT | YIDPYNX₇X₈AKYX₁₂X₁₃KFX₁₆G |
| | antibody, VH- | X₇ = D, R, L, K, F, S, Y or V |
| | CDR2 | X₈ = G, R, N, Q, E, L K, S, Y or V |
| | consensus | X₁₂ = N, A or S |
| | | X₁₃ = E or Q |
| | | X₁₆ = K or Q |
| 77 | Anti-TIGIT | GGPYGAYFDV |
| | antibody, VH- | |
| | CDR3 variant | |
| 78 | Anti-TIGIT | GGPYGDYFDV |
| | antibody, VH- | |
| | CDR3 variant | |
| 79 | Anti-TIGIT | GGPYGEYFDV |
| | antibody, VH- | |
| | CDR3 variant | |
| 80 | Anti-TIGIT | GGPYGFYFDV |
| | antibody, VH- | |
| | CDR3 variant | |
| 81 | Anti-TIGIT | GGPYGGYFDV |
| | antibody, VH- | |
| | CDR3 variant | |
| 82 | Anti-TIGIT | GGPYGIYFDV |
| | antibody, VH- | |
| | CDR3 variant | |
| 83 | Anti-TIGIT | GGPYGKYFDV |
| | antibody, VH- | |
| | CDR3 variant | |
| 84 | Anti-TIGIT | GGPYGNYFDV |
| | antibody, VH- | |
| | CDR3 variant | |
| 85 | Anti-TIGIT | GGPYGQYFDV |
| | antibody, VH- | |
| | CDR3 variant | |
| 86 | Anti-TIGIT | GGPYGRYFDV |
| | antibody, VH- | |
| | CDR3 variant | |
| 87 | Anti-TIGIT | GGPYGSYFDV |
| | antibody, VH- | |
| | CDR3 variant | |
| 88 | Anti-TIGIT | GGPYGTYFDV |
| | antibody, VH- | |
| | CDR3 variant | |
| 89 | Anti-TIGIT | GGPYGVYFDV |
| | antibody, VH- | |
| | CDR3 variant | |
| 90 | Anti-TIGIT | GGPYGYYFDV |
| | antibody, VH- | |
| | CDR3 variant | |
| 91 | Anti-TIGIT | GGPYGX₆YFDV |
| | antibody, VH- | X₆ = W, A, D, E, F, G, I, K, N, Q, R, S, |
| | CDR3 | T, V or Y |
| | consensus | |
| 92 | Anti-TIGIT | AAKTLAE |
| | antibody, VL- | |
| | CDR2 variant | |
| 93 | Anti-TIGIT | YAKTLAE |
| | antibody, VL- | |
| | CDR2 variant | |
| 94 | Anti-TIGIT | WAKTLAE |
| | antibody, VL- | |
| | CDR2 variant | |
| 95 | Anti-TIGIT | SAKTLAE |
| | antibody, VL- | |
| | CDR2 variant | |
| 96 | Anti-TIGIT | TAKTLAE |
| | antibody, VL- | |
| | CDR2 variant | |
| 97 | Anti-TIGIT | IAKTLAE |
| | antibody, VL- | |
| | CDR2 variant | |
| 98 | Anti-TIGIT | VAKTLAE |
| | antibody, VL- | |
| | CDR2 variant | |
| 99 | Anti-TIGIT | NNKTLAE |
| | antibody, VL- | |
| | CDR2 variant | |
| 100 | Anti-TIGIT | NIKTLAE |
| | antibody, VL- | |
| | CDR2 variant | |
| 101 | Anti-TIGIT | NLLTLAE |
| | antibody, VL- | |
| | CDR2 variant | |
| 102 | Anti-TIGIT | NTKTLAE |
| | antibody, VL- | |
| | CDR2 variant | |
| 103 | Anti-TIGIT | NVKTLAE |
| | antibody, VL- | |
| | CDR2 variant | |
| 104 | Anti-TIGIT | X₁X₂KTLAE |
| | antibody, VL- | X₁ = N, A, V, W, S, T, R, H G, I or V |
| | CDR2 | X₂ = A, N, I, L, T or V |
| | consensus | |
| 105 | Anti-CTLA4 | FISYDGNNKYYADSVKG |
| | CDRH2 | |
| | (Ipilimumab) | |
| 106 | Anti-CTLA4 | TGWLGPFDY |
| | CDRH3 | |
| | (Ipilimumab) | |
| 107 | Anti-CTLA4 | QVQLVESGGGVVQPGRSLRLSCAASGFTFSSYTMHW |
| | VH/VR in | VRQAPGKGLEWVTFISYDGNNKYYADSVKGRFTISR |
| | Heavy chain | DNSKNTLYLQMNSLRAEDTAIYYCARTGWLGPFDY |
| | (Ipilimumab) | WGQGTLVTVSS |
| 108 | Anti-CTLA4 | EIVLTQSPGTLSLSPGERATLSCRASQSVGSSYLAWY |
| | VL/VR in Light | QQKPGQAPRLLIYGAFSRATGIPDRFSGSGSGTDFTLT |
| | chain | ISRLEPEDFAVYYCQQYGSSPWTFGQGTKVEIK |
| | (Ipilimumab) | |
| 109 | Anti-CTLA4 | QVQLVESGGGVVQPGRSLRLSCAASGFTFSSYTMHW |
| | Heavy Chain | VRQAPGKGLEWVTFISYDGNNKYYADSVKGRFTISR |
| | (Ipilimumab) | DNSKNTLYLQMNSLRAEDTAIYYCARTGWLGPFDY |
| | | WGQGTLVTVSSASTKGPSVFPLAPSSKSTSGGTAALG |
| | | CLVKDYFPEPVTVSWNSGALTSGVHTFPAVLQSSGL |
| | | YSLSSVVTVPSSSLGTQTYICNVNHKPSNTKVDKRVE |
| | | PKSCDKTHTCPPCPAPELLGGPSVFLFPPKPKDTLMIS |
| | | RTPEVTCVVVDVSHEDPEVKFNWYVDGVEVHNAKT |
| | | KPREEQYNSTYRVVSVLTVLHQDWLNGKEYKCKVS |
| | | NKALPAPIEKTISKAKGQPREPQVYTLPPSRDELTKNQ |
| | | VSLTCLVKGFYPSDIAVEWESNGQPENNYKTTPPVLD |
| | | SDGSFFLYSKLTVDKSRWQQGNVFSCSVMHEALHNH |
| | | YTQKSLSLSPGK |
| 110 | Anti-CTLA4 | EIVLTQSPGTLSLSPGERATLSCRASQSVGSSYLAWY |
| | Light Chain | QQKPGQAPRLLIYGAFSRATGIPDRFSGSGSGTDFTLT |
| | (Ipilimumab) | ISRLEPEDFAVYYCQQYGSSPWTFGQGTKVEIKRTVA |
| | | APSVFIFPPSDEQLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |

### REFERENCES

Amaria R N, Prieto P A, Tetzlaff M T, Reuben A, Andrews M C, Ross M I, et al. Neoadjuvant plus adjuvant dabrafenib and trametinib versus standard of care in patients with high-risk, surgically resectable melanoma: a single-centre, open-label, randomised, phase 2 trial. Lancet Oncol. 2018 February; 19:181-93.
Amaria R N, Reddy S M, Tawbi H A, Davies M A, Ross M I, Glitza I C, et al. Neoadjuvant immune checkpoint blockade in high-risk resectable melanoma. Nat Med. 2018 November; 24:1649-54.
American Cancer Society. Cancer facts and figures 2016 [Internet]. Atlanta (Ga.): American Cancer Society, Inc.; 2016. Available from: http://www.cancer.org/acs/groups/content/@res earch/documents/document/acspc-047079.pdf.
Blank C U, Rozeman E A, Fanchi L F, Sikorska K, van de Wiel B, Kvistborg P, et al. Neoadjuvant versus adjuvant ipilimumab plus nivolumab in macroscopic stage III melanoma [letter]. Nat Med. 2018 November; 24:1655-61.
Carlsten M, Bjorkstrom N K, Norell H, Bryceson Y, van Hall T, Baumann B C, et al. DNAX accessory molecule-1 mediated recognition of freshly isolated ovarian carcinoma by resting natural killer cells. Cancer Res. 2007 Feb. 1; 67(3):1317-25.
Chauvin J M, Pagliano O, Fourcade J, Sun Z, Wang H, Sander C, et al. TIGIT and PD-1 impair tumor antigen-specific CD8+ T cells in melanoma patients. J Clin Invest. 2015 May; 125(5):2046-58.
Chemnitz J M, Parry R V, Nichols K E, June C H, Riley J L. SHP-1 and SHP-2 associate with immunoreceptor tyrosine-based switch motif of programmed death 1 upon primary human T cell stimulation, but only receptor ligation prevents T cell activation. J Immunol 2004; 173:945-54.
Clopper C J, Pearson E S. The use of confidence or fiducial limits illustrated in the case of the binomial. Biometrika 1934; 26(4):404-13.
Cross M J, Claesson-Welsh L. FGF and VEGF function in angiogenesis: signalling pathways, biological responses and therapeutic inhibition. Trends Pharmacol Sci. 2001 April; 22(4):201-7.
Dardalhon V, Schubart A S, Reddy J, Meyers J H, Monney L, Sabatos C A, et al. CD226 is specifically expressed on the surface of Th1 cells and regulates their expansion and effector functions. J Immunol. 2005 Aug. 1; 175(3):1558-65.
Disis M L. Immune regulation of cancer. J Clin Oncol 2010; 28(29):4531-8.
Dudley M E, Wunderlich J R, Yang J C, Sherry R M, Topalian S L, Restifo N P, et al. Adoptive cell transfer therapy following non-myeloablative but lymphodepleting chemotherapy for the treatment of patients with refractory metastatic melanoma. J Clin Oncol 2005; 23(10):2346-57.
Eggermont A M M, Blank C U, Mandala M, Long G V, Atkinson V, Dalle S, et al. Adjuvant pembrolizumab versus placebo in resected stage III melanoma. N Engl J Med. 2018 May 10; 378(19):1789-801.
Eggermont A M M, et al. Adjuvant Pembrolizumab versus Placebo in Resected State III Melanoma. N Engl J Med 2018; 378:1789-1801. DOI: 10.1056/NEJMoa1802357.
Ellis L M, Hicklin D J. VEGF-targeted therapy: mechanisms of anti-tumour activity. Nat Rev Cancer. 2008 August; 8:579-91.
Faries M B, Thompson J F, Cochran A J, Andtbacka R H, Mozzillo N, Zager J S, et al. Completion dissection or observation for sentinel-node metastasis in melanoma. N Engl J Med. 2017 Jun. 8; 376(23):2211-22.
Feng Y, Masson E, Dai D, Parker S M, Berman D, Roy A. Model-based clinical pharmacology profiling of ipilimumab in patients with advanced melanoma. Br J Clin Pharmacol. 2014 July; 78(1):106-17.
Ferrara N, Gerber H P, LeCouter J. The biology of VEGF and its receptors. Nat Med. 2003 June; 9(6):669-76.
Francisco L M, Sage P T, Sharpe A H. The PD-1 pathway in tolerance and autoimmunity. Immunol Rev 2010; 236:219-42.
Gershenwald J E, Scolyer R A, Hess K R, Sondak V K, Long G V, Ross M I, et al. Melanoma staging: evidence-based changes in the American Joint Committee on Cancer eighth edition cancer staging manual. C A Cancer J Clin. 2017 November; 67(6):472-92.
Hellmann M D, Rizvi N A, Goldman J W, Gettinger S N, Borghaei H, Brahmer J R, et al. Nivolumab plus ipilimumab as first-line treatment for advanced non-small-cell lung cancer (CheckMate 012): results of an open-label, phase 1, multicohort study. Lancet Oncol. 2016 Dec. 2. [Epub ahead of print].
Hirota T, Irie K, Okamoto R, Ikeda W, Takai Y. Transcriptional activation of the mouse Necl-5/Tage4/PVR/CD155 gene by fibroblast growth factor or oncogenic Ras through the Raf-MEK-ERK-AP-1 pathway. Oncogene. 2005 Mar. 24; 24(13):2229-35.
Hodi F S, Ribas A, Daud A, Hamid O, Robert C, Kefford R, et al. Patterns of response in patients with advanced melanoma treated with Pembrolizumab (MK-3475) and evaluation of immune-related response criteria (irRC). J Immunother Cancer. 2014; 2(Suppl 3):P103.
Hunder N N, Wallen H, Cao J, Hendricks D W, Reilly J Z, Rodmyre R, et al. Treatment of metastatic melanoma with autologous CD4+ T cells against NY-ESO-1. N Engl J Med 2008; 358(25):2698-703.
Jenkins R W, Barbie D A, Flaherty K T. Mechanisms of resistance to immune checkpoint inhibitors. Br J Cancer. 2018; 118:9-16.
Ji Y, Li Y, Bekele B N. Dose-finding in phase 1 clinical trials based on toxicity probability intervals. Clin Trials 2007; 4:235-44.
Ji Y, Wang S-J. Modified toxicity probability interval design: a safer and more reliable method than the 3+3 design for practical phase I trials. J Clin Oncol 2013; 31:1-12.
Ji Y, Liu P, Li Y, Bekele B N. A modified toxicity probability interval method for dose-finding trials. Clin Trials. 2010; 7:653-63.
Johnston R J, Comps-Agrar L, Hackney J, Yu X, Huseni M, Yang Y, et al. The immunoreceptor TIGIT regulates antitumor and antiviral CD8(+) T cell effector function. Cancer Cell. 2014 Dec. 8; 26(6):923-37.
Kato Y, Tabata K, Hori Y, Tachino S, Okamoto K, Matsui J, et al. Effects of lenvatinib on tumor-associated macrophages enhance antitumor activity of PD-1 signal inhibitors [abstract A92]. Poster session presented at: AACR-NCI-EORTC International Conference: Molecular Targets and Cancer Therapeutics; 2015 Nov. 5-6; Boston, Mass.
Larkin J, Ascierto P A, Dreno B, Atkinson V, Liszkay G, Maio M, et al. Combined vemurafenib and cobimetinib in BRAF-mutated melanoma. N Engl J Med. 2014 Nov. 13; 371(20):1867-76.
Levin S D, Taft D W, Brandt C S, Bucher C, Howard E D, Chadwick E M, et al. Vstm3 is a member of the CD28 family and an important modulator of T-cell function. Eur J Immunol. 2011 April; 41(4):902-15.
Lieu C, Heymach J, Overman M, Tran H, Kopetz S. Beyond VEGF: inhibition of the fibroblast growth factor pathway and antiangiogenesis. Clin Cancer Res. 2011 Oct. 1; 17(19):6130-9.
Limaverde-Sousa G, Steinberg C, Ferreira C G. Antiangiogenesis beyond VEGF inhibition: ajourney from antiangiogenic single-target to broad-spectrum agents. Cancer Treat Rev. 2014; 40:548-57.
Liu J, Blake S J, Yong M C R, Harjunpaa H, Ngiow S F, Takeda K, et al. Improved efficacy of neoadjuvant compared to adjuvant immunotherapy to eradicate metastatic disease. Cancer
Discov. 2016 December; 6(12):13 Long G V, Atkinson V, Cebon J S, Jameson M B, Fitzharris B M, McNeil C M, et al. Standard-dose pembrolizumab in combination with reduced-dose ipilimumab for patients with advanced melanoma (KEYNOTE-029): an open-label, phase 1b trial. Lancet Oncol. 2017 September; 18:1202-10.
Makker V, Rasco D, Vogelzang N J, Messing M, Brose M S, Cohn A L, et al. Lenvatinib+Pembrolizumab in patients with advanced endometrial cancer: updated results. Poster session presented at: 2018 American Society of Clinical Oncology (ASCO) Annual Meeting; 2018 Jun. 1-5; Chicago, Ill. Poster no. 5596.
Masson D, Jarry A, Baury B, Blanchardie P, Laboisse C, Lustenberger P, et al. Overexpression of the CD155 gene in human colorectal carcinoma. Gut. 2001 August; 49(2):236-40.
MedPage Today [Internet]. New York (N.Y.): MedPage Today, LLC; c2019. Neoadjuvant anti-PD-1 predicts melanoma outcomes—one-dose immune response tied to 63% DFS and 93% OS at 2 years [press release]. 2019 Feb. 27 [cited 2019 May 23]; [6 screens]. Available from: https://www.medpagetoday.com/hematologyon cology/skincancer/78256.
Merck Inventing For Life [Internet]. Kenilworth (NJ): Merck & Co., Inc.; c2009-2018. European Medicines Agency adopts positive opinion for Merck's KEYTRUDA (pembrolizumab) for six-week dosing schedule across all current monotherapy indications [press release]. 2019 Mar. 4 [cited 2019 Apr. 17]; 7 p. Available from: https://www.mrknewsroom.com/news-release/oncology/european-medicines-agency-adopts-positive-opinion-mercks-keytruda-pembrolizuma.
O'Donnell J S, Long G V, Scolyer R A, Teng M W L, Smyth M J. Resistance to PD1/PDL1 checkpoint inhibition. Cancer Treat Rev. 2017; 52:71-81.
Okazaki T, Maeda A, Nishimura H, Kurosaki T, Honjo T. PD-1 immunoreceptor inhibits B cell receptor-mediated signaling by recruiting src homology 2-domain-containing tyrosine phosphatase 2 to phosphotyrosine. Proc Natl Acad Sci USA 2001; 98(24):13866-71.
Olson D, Luke J J, Hallmeyer S, Bajaj M, Carll T, Krausz T, et al. Phase II trial of pembrolizumab (pembro) plus 1 mg/kg ipilimumab (ipi) immediately following progression on anti-PD-1 Ab in melanoma (mel). Presented at: 2018 American Society of Clinical Oncology (ASCO) Annual Meeting; 2018 Jun. 1-5; Chicago, Ill. J Clin Oncol. 2018; 36(15 suppl). Abstract no. 9514.
Parry R V, Chemnitz J M, Frauwirth K A, Lanfranco A R, Braunstein I, Kobayashi S V, et al. CTLA-4 and PD-1 receptors inhibit T-cell activation by distinct mechanisms. Mol Cell Biol 2005; 25(21):9543-53.
Pitt J M, Vetizou M, Daillere R, Roberti M P, Yamazaki T, Routy B, et al. Resistance mechanisms to immune-checkpoint blockade in cancer: tumor-intrinsic and -extrinsic factors. Immunity. 2016 Jun. 21; 44:1255-69.
Postow M A, Chesney J, Pavlick A C, Robert C, Grossmann K, McDermott D, et al. Nivolumab and ipilimumab versus ipilimumab in untreated melanoma. N Engl J Med. 2015 May 21; 372(21):2006-17.
Restifo N P, Smyth M J, Snyder A. Acquired resistance to immunotherapy and future challenges. Nat Rev Cancer. 2016 February; 16:121-6.
Riley J L. PD-1 signaling in primary T cells. Immunol Rev 2009; 229:114-25. Robert C, Karaszewska B, Schachter J, Rutkowski P, Mackiewicz A, Stroiakovski D, et al. Improved overall survival in melanoma with combined dabrafenib and trametinib. N Engl J Med. 2015 Jan. 1; 372(1):30-9.
Robert C, Long G V, Brady B, Dutriaux C, Maio M, Mortier L, et al. Nivolumab in previously untreated melanoma without BRAF mutation. N Engl J Med. 2015 Jan. 22; 372(4):320-30.
Robert C, Schachter J, Long G V, Arance A, Grob J J, Mortier L, et al. Pembrolizumab versus Ipilimumab in Advanced Melanoma. N Engl J Med. 2015 Jun. 25; 372(26):2521-32.
Robert C, Schachter J, Long G V, Arance A, Grob J J, Mortier L, et al. 5-year survival and other long-term outcomes from the KEYNOTE-006 study of pembrolizumab for ipilimumab-naive advanced melanoma. Poster session presented at: 2019 American Association for Cancer Research (AACR) Annual Meeting; 2019 Mar. 29-Apr. 3; Atlanta, Ga. Poster no. CT188.
Seymour L, Bogaerts J, Perrone A, Ford R, Schwartz L H, Mandrekar S, et al. iRECIST: guidelines for response criteria for use in trials testing immunotherapeutics. Lancet Oncol. 2017 March; 18(3):e143-52.
Sharma P, Hu-Lieskovan S, Wargo J A, Ribas A. Primary, adaptive, and acquired resistance to cancer immunotherapy. Cell. 2017 Feb. 9; 168:707-23
K-A, Fitz L J, Lee J M, Benander C, George J A, Wooters J, et al. PD-1 inhibits T-cell receptor induced phosphorylation of the ZAP70/CD3zeta signalosome and downstream signaling to PKCtheta. FEBS Lett. 2004; 574:37-41.
Siegel R, Naishadham D, Jemal A. Cancer statistics, 2012. CA Cancer J Clin. 2012 January-February; 62(1):10-29.
Stanietsky N, Rovis T L, Glasner A, Seidel E, Tsukerman P, Yamin R, et al. Mouse TIGIT inhibits NK-cell cytotoxicity upon interaction with PVR. Eur J Immunol. 2013 August; 43(8):2138-50.
Tammela T, Alitalo K. Lymphangiogenesis: molecular mechanisms and future promise. Cell. 2010 Feb. 19; 140:460-76.
Sosman J A. Overview of the management of advanced cutaneous melanoma [Internet]. Waltham (Mass.): UpToDate, Inc.; c2015 [last updated 2015 Nov. 17]. Available from: http://www.uptodate.com/contents/overview-of-the-management-of-advanced-cutaneous-melanoma.
Valsecchi M E. Combined nivolumab and ipilimumab or monotherapy in untreated melanoma [correspondence]. N Engl J Med. 2015 Sep. 24; 373(13):1270-71.
Weber J, Mandala M, Del Vecchio M, Gogas H J, Arance A M, Cowey C L, et al. Adjuvant nivolumab versus ipilimumab in resected stage III or IV melanoma. N Engl J Med. 2017 Nov. 9; 377(19):1824-35.
WHO Health Organization. GLOBOCAN 2012: Country Fast Stat: European Union (Fact sheet No. EU-28). 2012.
Wolchok J D, Hoos A, O'Day S, Weber J S, Hamid O, LebbeC, et al. Guidelines for the evaluation of immune therapy activity in solid tumors: immune-related response criteria. Clin Cancer Res 2009; 15(23):7412-20.
Wolchok J D, Chiarion-Sileni V, Gonzalez R, Rutkowski P, Grob J J, Cowey C L, et al. Overall survival with combined nivolumab and ipilimumab in advanced melanoma. N Engl J Med. 2017 Oct. 5; 377(14):1345-56.
Yamamoto Y, Matsui J, Matsushima T, Obaishi H, Miyazaki K, Nakamura K, et al. Lenvatinib, an angiogenesis inhibitor targeting VEGFR/FGFR, shows broad antitumor activity in human tumor xenograft models associated with microvessel density and pericyte coverage. Vasc Cell. 2014; 6:18.
Zhang X, Schwartz J-C D, Guo X, Bhatia S, Cao E, Chen L, et al. Structural and functional analysis of the costimulatory receptor programmed death-1. Immunity 2004; 20:337-47.
Zimmer L, Apuri S, Eroglu Z, Kottschade L A, Forschner A, Gutzmer R, et al. Ipilimumab alone or in combination with nivolumab after progression on anti-PD-1 therapy in advanced melanoma. Eur J Cancer. 2017; 75:47-55.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method of treating cancer, an infection disease or an infection comprising administering to a subject in need thereof effective amounts of:
(a) an anti-human PD-1 monoclonal antibody or antigen binding fragment thereof,
(b) an anti-human CTLA4 monoclonal antibody or antigen binding fragment thereof; and
(c) an anti-human TIGIT monoclonal antibody or antigen binding fragment thereof.

2. The method of claim 1, wherein the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer, non-small cell lung cancer, gastric cancer, colorectal cancer, cervical cancer, synovial sarcoma, head and neck cancer, squamous cell carcinoma, lymphoma, diffuse large B-cell lymphoma, non-Hodgkin lymphoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

3-6. (canceled)

7. A pharmaceutical composition, a kit, or a therapeutic combination for treating cancer, an infection disease, or an infection in a subject in need thereof comprising:
(a) an anti-human PD-1 monoclonal antibody or antigen binding fragment thereof;
(b) an anti-human CTLA4 monoclonal antibody or antigen binding fragment thereof; and
(c) an anti-human TIGIT monoclonal antibody or antigen binding fragment thereof.

8-14. (canceled)

15. The method of claim 1, wherein the anti-human PD-1 monoclonal antibody, the anti-human CTLA4 monoclonal antibody, and/or the anti-human TIGIT monoclonal antibody is a human antibody.

16. The method of claim 1, wherein the anti-human PD-1 monoclonal antibody, the anti-human CTLA4 monoclonal antibody, and/or the anti-human TIGIT monoclonal antibody is a humanized antibody.

17-20. (canceled)

21. The method of claim 1, wherein the anti-human PD-1 monoclonal antibody or antigen binding fragment thereof comprises a light chain variable region (V_{L}) complementarity determining region (CDR) 1, a V_{L} CDR2, and a V_{L} CDR3 comprising amino acid sequences as set forth in SEQ ID NOS:1, 2, and 3, respectively, and a heavy chain variable region (V_{H}) CDR1, a V_{H} CDR2, and a V_{H} CDR3 comprising amino acid sequences as set forth in SEQ ID NOS:6, 7, and 8, respectively.

22. The method of claim 1, wherein the anti-human PD-1 monoclonal antibody or antigen binding fragment thereof comprises a V_{L} comprising an amino acid sequence as set forth in SEQ ID NO:4, and a V_{H} comprising an amino acid sequence as set forth in SEQ ID NO: 9.

23. The method of claim 1, wherein the anti-human PD-1 monoclonal antibody or antigen binding fragment thereof comprises a light chain comprising or consisting of an amino acid sequence as set forth in SEQ ID NO:5 and a heavy chain comprising or consisting of an amino acid sequence as set forth in SEQ ID NO:10.

24-26. (canceled)

27. The method of claim 1, wherein the anti-human PD-1 monoclonal antibody or antigen binding fragment thereof is pembrolizumab.

28. The method of claim 1, wherein the anti-human PD-1 monoclonal antibody or antigen binding fragment thereof is nivolumab or cemiplimab.

29. (canceled)

30. The method of claim 1, wherein the anti-human CTLA4 monoclonal antibody or antigen binding fragment thereof comprises:
(a) a V_{L} CDR1 comprising the amino acid sequence of SEQ ID NO: 14, a V_{L} CDR2 comprising the amino acid sequence of SEQ ID NO: 15, and a V_{L} CDR3 comprising the amino acid sequences as set forth in SEQ ID NOS:16, 17, or 18, and a V_{H} CDR1, a V_{H} CDR2, and a V_{H} CDR3 comprising amino acid sequences as set forth in SEQ ID NOS:11, 12, and 13, respectively; or
(b) a V_{L} CDR1, a V_{L} CDR2, and a V_{L} CDR3 comprising the amino acid sequences as set forth in SEQ ID NOS:54, 55, and 56, respectively, and a V_{H} CDR1, a V_{H} CDR2, and a V_{H} CDR3 comprising amino acid sequences as set forth in SEQ ID NOS:57, 105, and 106, respectively.

31. The method of claim 1, wherein the anti-human CTLA4 monoclonal antibody or antigen binding fragment thereof comprises:
(a) a V_{L} comprising an amino acid sequence as set forth in SEQ ID NO:20, and a V_{H} region comprising an amino acid sequence as set forth in SEQ ID NO:19;
(b) a V_{L} comprising an amino acid sequence as set forth in SEQ ID NO:42, and a V_{H} region comprising an amino acid sequence as set forth in SEQ ID NO:41;
(c) a V_{L} comprising an amino acid sequence as set forth in SEQ ID NO:44, and a V_{H} region comprising an amino acid sequence as set forth in SEQ ID NO: 43;
(d) a V_{L} comprising an amino acid sequence as set forth in SEQ ID NO:44, and a V_{H} region comprising an amino acid sequence as set forth in SEQ ID NO: 45;
(e) a V_{L} comprising an amino acid sequence as set forth in SEQ ID NO:47, and a V_{H} region comprising an amino acid sequence as set forth in SEQ ID NO:46;
(f) a V_{L} comprising an amino acid sequence as set forth in SEQ ID NO:49, and a V_{H} region comprising an amino acid sequence as set forth in SEQ ID NO:48;
(g) a V_{L} comprising an amino acid sequence as set forth in SEQ ID NO:51, and a V_{H} region comprising an amino acid sequence as set forth in SEQ ID NO:50; or
(h) a V_{L} comprising an amino acid sequence as set forth in SEQ ID NO:108, and a V_{H} region comprising an amino acid sequence as set forth in SEQ ID NO:107.

32. The method of claim 1, wherein the anti-human CTLA4 monoclonal antibody or antigen binding fragment thereof comprises:
(a) a light chain comprising or consisting of an amino acid sequence as set forth in SEQ ID NO:53 and a heavy chain comprising or consisting of an amino acid sequence as set forth in SEQ ID NO:52; or
(b) a light chain comprising or consisting of an amino acid sequence as set forth in SEQ ID NO:110 and a heavy chain comprising or consisting of an amino acid sequence as set forth in SEQ ID NO:109.

33. The method of claim 1, wherein the anti-human TIGIT monoclonal antibody or antigen binding fragment thereof comprises a V_{L} CDR1, a V_{L} CDR2, and a V_{L} CDR3 comprising amino acid sequences as set forth in SEQ ID NOS:21, 22, and 23, respectively, and a V_{H} CDR1, a V_{H} CDR2, and a V_{H} CDR3 comprising amino acid sequences as set forth in SEQ ID NOS:26, 27, and 28, respectively.

34. The method of claim 1, wherein the anti-human TIGIT monoclonal antibody or antigen binding fragment thereof comprises a V_{L} comprising an amino acid sequence as set forth in SEQ ID NO:24, and a V_{H} comprising an amino acid sequence as set forth in SEQ ID NO:29.

35. The method of claim 1, wherein the anti-human TIGIT monoclonal antibody or antigen binding fragment thereof comprises a light chain comprising or consisting of an amino acid sequence as set forth in SEQ ID NO:25 and a heavy chain comprising or consisting of an amino acid sequence as set forth in SEQ ID NO:30.

36-59. (canceled)

60. The method of claim 27, wherein 200 mg pembrolizumab is administered once every three weeks, or 400 mg pembrolizumab is administered once every six weeks.

61. (canceled)

62. The method of claim 1, wherein 200 mg of the anti-human TIGIT monoclonal antibody or antigen binding fragment thereof is administered, and wherein the anti-human TIGIT monoclonal antibody or antigen binding fragment thereof is administered every three weeks.

63. The method of claim 1, wherein 25 mg of the anti-human CTLA4 monoclonal antibody or antigen binding fragment thereof is administered, and where the anti-human CTLA4 monoclonal antibody or antigen binding fragment thereof is administered every six weeks.

64-88. (canceled)

89. The method claim 1, wherein the cancer is metastatic, locally advanced or resectable, relapsed, and/or relapsed.

90-92. (canceled)

93. The method of claim 92, wherein:
(i) the cancer is refractory melanoma and has progressed on treatment with an anti-PD-1/L1 mAb administered either as monotherapy, or in combination with a checkpoint inhibitor or other therapy,
(ii) the subject has received at least 2 doses of an approved anti-PD-1/L1 monoclonal antibody:
(iii) the subject has demonstrated disease progression after PD-1/L1 treatment as defined by RECIST 1.1: or
(iv) the subject has at least one melanoma selected from the group consisting of: PD-1 refractory melanoma, first-line advanced melanoma, and neoadjuvant melanoma.

94-96. (canceled)

97. The method claim 1, wherein the anti-human PD-1 monoclonal antibody or antigen binding fragment, the anti-human TIGIT monoclonal antibody or antigen binding fragment, and the anti-human CTLA4 monoclonal antibody or antigen binding fragment are administered contemporaneously.

98-99. (canceled)

100. The method of claim 1, further comprising administering to the subject at least one therapeutic agent and/or active agent, wherein the therapeutic agent and/or active agent comprises an antibody or fragment thereof, an immunomodulator, a hormone, a cytotoxic agent, an enzyme, a radionuclide, a second antibody conjugated to at least one immunomodulator, enzyme, radioactive label, hormone, antisense oligonucleotide, biotherapeutic agent, chemotherapeutic agent, cytotoxic agent, or a combination thereof.
